(19) United States
(12) Patent Application Publication
(10) Pub. No.: US 2019/0270797 A1
(43) Pub. Date: Sep. 5, 2019

(54) CREBBP RELATED CANCER THERAPY

(71) Applicant: EPIZYME, INC., Cambridge, MA (US)

(72) Inventors: Alexandra GRASSIAN, Cambridge, MA (US); Scott RIBICH, Cambridge, MA (US); Jesse SMITH, Cambridge, MA (US); Darren Martin HARVEY, Acton, MA (US)

(21) Appl. No.: 16/320,447

(22) PCT Filed: Jul. 25, 2017
(86) PCT No.: PCT/US2017/043757

§ 371 Date: Jan. 24, 2019

Related U.S. Application Data
(60) Provisional application No. 62/366,249, filed on Jul. 25, 2016.

(51) Int. Cl. C07K 16/22; G01N 33/50; A61P 35/00; A61K 31/7105; A61K 31/711

(52) U.S. Cl. CPC C07K 16/22; G01N 33/5041; A61K 31/711; A61K 31/7105; A61P 35/00

(57) ABSTRACT

The present disclosure provides novel cancer therapies. The treatment of cancers harboring EP300 mutations with CREBBP inhibition therapy is described.

### BACKGROUND

There is a need to develop improved therapies for the treatment of cancer. Mutation status of an individual can provide an opportunity for unique treatment options.

### SUMMARY

The present disclosure provides certain therapies useful for the treatment of cancer. Methods and compositions provided by the present disclosure may be applicable to treatment of a wide range of solid tumors and/or to hematological malignancies.

Some aspects of the present disclosure provide that CREBBP may be a therapeutic target which shows selective sensitivity. For example, the present disclosure demonstrates that sensitivity to CREBBP inhibition therapy, e.g. sensitivity to treatment with a CREBBP antagonist, is associated with reduced level and/or activity of EP300. The present disclosure specifically demonstrates, in some embodiments, that sensitivity to CREBBP inhibition therapy is associated with the presence of one or more loss-of-function mutations and/or deletions in EP300.

Furthermore, the present disclosure establishes that reduction in EP300 level and/or activity is observed at high frequency in a variety of different tumor types. For example, the present disclosure documents detection of particular EP300 variants (e.g., certain loss-of-function mutations and/or deletions in EP300 variants) in tumors of various different types.

According to certain embodiments of the present disclosure, administration of CREBBP inhibition therapy is useful for the treatment of certain cancers, and may be particularly effective to treat cancer in subjects harboring an EP300 variant.

In some embodiments, the present disclosure teaches that administration of CREBBP inhibition therapy can decrease level and/or activity of a CREBBP gene or gene product. In some embodiments, CREBBP inhibition therapy comprises administration of a CREBBP antagonist. In some embodiments, CREBBP inhibition therapy reduces tumor volume. In some embodiments, CREBBP inhibition therapy reduces a rate and/or extent of tumor growth over a period of time.

In some embodiments, a CREBBP antagonist may be of any chemical class. For example, in some embodiments a CREBBP antagonist may comprise one or more small molecule, polypeptide (e.g., antibodies), and/or nucleic acid agents. In some embodiments, a nucleic acid CREBBP antagonist may comprise an oligonucleotide (e.g., an antisense oligonucleotide, an siRNA, an shRNA, or an miRNA); in some embodiments, a nucleic acid CREBBP antagonist may comprise a genetic modifying agent (e.g., an agent that mediates gene editing or other gene therapy such as, for example, one or more components of a gene editing system such as a clustered regularly interspaced short palindromic repeats (CRISPR)/Cas system, a transcription activator-like effector nuclease (TALEN), or a zinc finger nuclease).

In some embodiments, an EP300 mutation manifests as, is detectable as, and/or is characterized by one or more of a genetic mutation or an epigenetic mark. In some embodiments, an EP300 mutation manifests as, is detectable as, and/or is characterized by a decreased level and/or activity of an EP300 gene or gene product (e.g., a transcript or polypeptide relative to an appropriate reference. In some embodiments, an EP300 mutation manifests as, is detectable as, and/or is characterized by presence or level of a particular form of an EP300 gene or gene product. In some embodiments, an EP300 mutation comprises a frame shift mutation, a splice variant, a missense mutation, a nonsense mutation, an insertion, an inversion, a deletion, or a combination thereof. In some embodiments, an EP300 mutation may comprise an alteration at a site that is upstream, downstream, or within the EP300 coding region. In some embodiments, an EP300 mutation may comprise an alteration at a site that is upstream, downstream, or within the HAT domain of EP300. In some embodiments, an EP300 mutation may comprise an alteration at a site that is within an EP300 regulatory region (e.g., a promoter, enhancer, splice site, or termination site).

The summary above is meant to illustrate, in a non-limiting manner, some of the embodiments, advantages, features, and uses of the technology disclosed herein. Other embodiments, advantages, features, and uses of the technology disclosed herein will be apparent from the Drawings, the Detailed Description, the Examples, and the Claims.

### BRIEF DESCRIPTION OF THE DRAWINGS

FIG. 1 shows sensitivity of various tumor cell lines to loss or inhibition of CREBBP.

FIGS. 2A and 2B show sensitivity to loss or inhibition of CREBBP in different tumor types.

FIGS. 3A-3D demonstrate sensitivity to loss or inhibition of CREBBP in EP300 mutant cancer cells.

FIGS. 4A and 4B demonstrates that mutations in EP300 are common in a variety of cancers.

FIG. 5 demonstrates that some cell lines with CREBBP mutations are sensitive to EP300 loss

FIG. 6 is a depiction of a representative wild type CREBBP/EP300 protein, representative domain localizations, and protein interactions.

FIGS. 7A-7C further demonstrate sensitivity to loss or inhibition of CREBBP in EP300 mutant cancer cells.

FIG. 8 further demonstrates that EP300 mutations correlate with sensitivity to inhibition of CREBBP.

### DEFINITIONS

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Allele: As used herein, the term “allele” refers to one of two or more existing genetic variants of a specific polymorphic genomic locus.

Amino acid: As used herein, the term “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in one or more certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog can be generated through performance of a synthetic process different from that used to generate the reference substance.

Antagonist: As used herein, the term “antagonist” may refer to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (for example when the one or more values of interest define a sufficiently narrow range that application of such a percentage variance would obviate the stated range).

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Chromosome: As used herein, the term “chromosome” refers to a DNA molecule, optionally together with associated polypeptides and/or other entities, for example as found in the nucleus of eukaryotic cells. Typically, a chromosome carries genes and functions (e.g., origin of replication) that permit it to transmit hereditary information.

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at position 190 in the reference polypeptide; those of ordinary skill in the art readily appreciate how to identify “corresponding” amino acids (see. e.g., Benson et al. Nucl. Acids Res. (1 Jan. 2013) 41 (D1): D36-D42; Pearson et al. PNAS Vol. 85, pp. 2444-2448, April 1988). Those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Domain: As used herein the term “domain” refers to a section or portion of a polypeptide. In some embodiments, a “domain” is associated with a particular structural and/or functional feature of the polypeptide so that, when the domain is physically separated from the rest of its parent polypeptide, it substantially or entirely retains the particular structural and/or functional feature. In some embodiments, a domain may include a portion of a polypeptide that, when separated from that (parent) polypeptide and linked with a different (recipient) polypeptide, substantially retains and/or imparts on the recipient polypeptide one or more structural and/or functional features that characterized it in the parent polypeptide. In some embodiments, a domain is a section of a polypeptide. In some such embodiments, a domain is characterized by a particular structural element (e.g., a particular amino acid sequence or sequence motif, α-helix character, β-sheet character, coiled-coil character, random coil character), and/or by a particular functional feature (e.g., binding activity, enzymatic activity, folding activity, signaling activity

Epigenetic Mark: As used herein, the term “epigenetic mark” refers to a feature of a nucleic acid or polypeptide not directly governed by genetic code. For example, in some embodiments, an epigenetic mark may represent or result from a modification to the nucleic acid or polypeptide. In some embodiments, such modification can include, for example, methylation, acetylation, ubiquitiniation, phosphorylation, ribosylation, amidation, glycosylation or combinations thereof.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gene: As used herein, the term “gene” refers to a DNA sequence in a chromosome that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression).

Mutant: As used herein, the term “mutant” refers to an organism, a cell, or a biomolecule (e.g., a nucleic acid or a protein) that comprises a genetic variation as compared to a reference organism, cell, or biomolecule. For example, a mutant nucleic acid may, in some embodiments, comprise a mutation, e.g., a nucleobase substitution, a deletion of one or more nucleobases, an insertion of one or more nucleobases, an inversion of two or more nucleobases, as, or a truncation, as compared to a reference nucleic acid molecule. Similarly, a mutant protein may comprise an amino acid substitution, insertion, deletion, inversion, or truncation, as compared to a reference polypeptide. Additional mutations, e.g., fusions and indels, are known to those of skill in the art. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of the enzymatic activity of an enzyme, or a partial loss of function, e.g., a diminished enzymatic activity of an enzyme. In some embodiments, a mutant comprises a genetic variant that is associated with a gain of function, e.g., with a negative or undesirable alteration in a characteristic or activity in a gene product. In some embodiments, a mutant is characterized by a reduction or loss in a desirable level or activity as compared to a reference; in some embodiments, a mutant is characterized by an increase or gain of an undesirable level or activity as compared to a reference. In some embodiments, the reference organism, cell, or biomolecule is a wild-type organism, cell, or biomolecule.

Nucleic acid: As used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguano sine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Peptide: As used herein, the term “peptide” refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and substantially pyrogen-free.

Polypeptide: As used herein, the term “polypeptide,” which is interchangeably used herein with the term “protein,” refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” refers to a standard or control relative to which a comparison is performed. For example, in some embodiments, an agent, animal, individual, population, sample, sequence, or value of interest is compared to a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a source of interest, as described herein. In some embodiments, a source of interest comprises an organism, such as a microbe, a plant, an animal or a human. In some embodiments, a biological sample comprises biological tissue or fluid. In some embodiments, a biological sample may comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a biological sample comprises cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or polypeptides extracted from a sample or obtained by subjecting a primary sample to techniques such as amplification or reverse transcription of mRNA, isolation and/or purification of certain components.

Single Nucleotide Polymorphism (SNP): As used herein, the term “single nucleotide polymorphism” or “SNP” refers to a particular base position in the genome where alternative bases are known to distinguish one allele from another. In some embodiments, one or a few SNPs and/or “copy number polymorphisms” “CNPs” is/are sufficient to distinguish complex genetic variants from one another so that, for analytical purposes, one or a set of SNPs and/or CNPs may be considered to be characteristic of a particular variant, trait, cell type, individual, species, or set thereof. In some embodiments, one or a set of SNPs and/or CNPs may be considered to define a particular variant, trait, cell type, individual, species, or set thereof.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, therapeutic agents may be CREBBP antagonists as described herein.

Therapeutically effective amount: As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor. Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Wild-type: As used herein, the term “wild-type” refers to a form of an entity (e.g., a polypeptide or nucleic acid) that has a structure and/or activity as found in nature in a “normal” (as contrasted with mutant, diseased, altered) state or context. In some embodiments, more than one “wild type” form of a particular polypeptide or nucleic acid may exist in nature, for example as “alleles” of a particular gene or normal variants of a particular polypeptide. In some embodiments, that form (or those forms) of a particular polypeptide or nucleic acid that is most commonly observed in a population (e.g., in a human population) is the “wild type” form.

### DETAILED DESCRIPTION OF CERTAIN EMBODIMENTS

Some aspects of the present disclosure are based on the recognition of the importance of histone acetyl transferases, such as CREBBP and EP300, in initiation and/or progression of cancer. Some aspects of the present disclosure encompass the recognition that histone acetyl transferases represent a valuable target for cancer therapies. Some aspects of this disclosure are based on the recognition that CREBBP activity in cancer cells comprising a mutant EP300 sequence is important for survival and/or proliferation of the cells. Some aspects of this disclosure provide methods and strategies for inhibiting the survival and/or proliferation of malignant cells comprising a mutant EP300 sequence by contacting such cells with a CREBBP inhibitor, e.g., by contacting such cells with a CREBBP inhibitor in vitro, or in vivo, e.g., by administering a CREBBP inhibitor to a subject harboring such cells or a tumor comprising such cells.

Some aspects of the present disclosure provide that CREBBP is a therapeutic target in various cancers, and that such cancers exhibit selective sensitivity to treatment with a CREBBP inhibitor. For example, some aspects of this disclosure provide that certain cancers comprising a mutant EP300 sequence associated with an EP300 loss-of-function are sensitive to treatment with a CREBBP inhibitor and that growth, proliferation, and/or survival of such mutant cancer cells can effectively be inhibited or abolished by contacting such cells with a CREBBP inhibitor in vitro and in vivo. The present disclosure also teaches that sensitivity to CREBBP inhibition therapy, e.g. CREBBP antagonists, is observed in a variety of indications. Some aspects of the present disclosure are based on the recognition that sensitivity to CREBBP inhibition therapy is associated with loss-of-function mutations or DNA deletions in EP300. Some aspects of the present disclosure are based on the recognition that EP300 is mutated at a high frequency across many tumor types and that such mutant tumors can be treated with CREBBP inhibition therapy.

In some embodiments, the present disclosure teaches that administration of CREBBP inhibition therapy can decrease a level and/or an activity of a CREBBP gene or gene product. In some embodiments, CREBBP inhibition therapy comprises administration of a CREBBP antagonist, e.g., of a CREBBP antagonist provided herein. In some embodiments, a CREBBP antagonist may be of any chemical class. For example, in some embodiments a CREBBP antagonist may comprise a small molecule, a peptide, an antibody, or a nucleic acid. In some embodiments, a nucleic acid CREBBP antagonist may comprise an oligonucleotide (e.g., an antisense oligonucleotide), an siRNA, an shRNA, an miRNA, or a genetic modifying agent (e.g., that mediates gene editing or other gene therapy for example CRISPR, TALENS, zinc finger nucleases). In some embodiments, CREBBP inhibition therapy reduces tumor volume. In some embodiments, CREBBP inhibition therapy reduces a rate and/or extent of tumor growth over a period of time.

In some embodiments, the present disclosure provides methods comprising administration of CREBBP inhibition therapy to a subject suffering from a cancer determined to harbor at least one mutation in EP300.

In some embodiments, the present disclosure provides methods for identifying a subject as a candidate for administration of CREBBP therapy based on the subject's EP300 mutation status. In some embodiments, the present disclosure provides methods for determining that a tumor in a subject is sensitive to treatment with a CREBBP inhibitor based on the EP300 mutation status of the tumor or of a cell comprised in the tumor. In some embodiments, the method comprises detecting a loss-of-function mutation in an EP300 gene in the subject. In some embodiments, the subject is sensitive to CREBBP therapy, if the subject, a tumor within the subject, or a cell comprised in such a tumor, is determined to harbor a loss-of-function mutation in an EP300 gene. In some embodiments, the method further comprises administering CREBBP inhibition therapy to the subject, e.g., based on the subject being identified as sensitive to CREBBP inhibition therapy.

### Acetyl Transferases

Histone acetylation and deacetylation are processes by which lysine residues within the N-terminal tail protruding from histone cores of the nucleosome are acetylated and deacetylated. Without wishing to be bound by any particular theory, it is believed that histone acetylation is a part of gene regulation. Histone Acetyltransferases, also known as HATs or KATs for Lysine Acetyltransferases, are a family of enzymes that acetylate the histone tails of the nucleosome among other nuclear and cytoplasmic non-histone targets.

KATs can be divided into families based on their structure and sequence similarity. KAT families include, for example, the Gcn5-related N-acetyltransferase (GNAT) family, which includes GCN5 and PCAF, the CREBBP/EP300 family and the MYST (MOZ, Ybf2/Sas3, Sas2, Tip60) family, which includes Tat interacting protein, 60 kDa (Tip60), monocytic leukemia zinc finger protein/MOZ-related factor protein (MOZ/MORF). Different KATs may contain various other domains in addition to the HAT domain which facilitate interactions with other proteins, including reader domains for acetylation and other modifications. See, e.g., Farria et al. Oncogene (2015) 34, 4901-4913, incorporated herein by reference. Some KATs, for example those in the GNAT and CREBBP/EP300 families, contain bromodomains. Bromodomains help KATs recognize and bind to acetylated lysine residues on histone substrates. Together these domains allow for specificity and diversity in KAT substrates. All KATs examined to date have important functions in cellular differentiation and embryo development. Several KATs have also been associated with oncogenesis. For example, CREBBP/EP300, have been implicated in cancer development and progression. See, e.g., Farria et al. Oncogene (2015) 34, 4901-4913; Lee et al. Nat. Rev. Mol. Cell Biol. 8 (4): 284-95; and Avvakumov et al. Oncogene (2007) 26, 5395-5407, the entire contents of each of which are incorporated herein by reference.

### CREBBP/EP300

Transcriptional coactivators CREB binding protein (referred to herein as CREBBP or CBP) and E1A binding protein p300 (referred to herein as EP300 or p300) are important regulators of RNA polymerase II-mediated transcription. Studies indicate that the ability of these multidomain proteins to acetylate histones and other proteins is critical for many biological processes. CREBBP and EP300 have been reported to interact with more than 400 different cellular proteins, including factors important to cancer development and progression such as hypoxia-inducible factors-1 (HIF-1), beta-catenin, c-Myc, c-Myb, CREB, E1, E6, p53, AR and estrogen receptor (ER). See, e.g., Kalkhoven et al. Biochemical Phamacology 68 (2004) pg. 1145-1155; and Farria et al. Oncogene (2015) 34, 4901-4913.

Genetic alterations in genes encoding CREBBP and EP300 and their functional inactivation have been linked to human disease. Furthermore, despite their high degree of homology, CREBBP and EP300 are not completely redundant but also have unique roles in cellular function.

CREBBP/EP300 have been implicated in processes of DNA replication and DNA repair. CREBBP/EP300 have also been implicated in regulation of progression of the cell cycle, ubiquitination and degradation of p53 transcription factor, and regulation of nuclear import. Due to these numerous roles mutations in the gene or changes in the expression level, activity or localization of CREBBP or EP300 can result in disease state. See, e.g., Vo et. al. J Biol Chem. 2001 Apr. 27; 276(17):13505-8; and Chan et. al. Journal of Cell Science 2001 114: 2363-2373, the entire contents of each of which are incorporated herein by reference. Diseases that could result from such alterations in CREBP or EP300 can include but are not limited to developmental disorders, for example Rubionstein-Taybi syndrome (RTS), progressive neurodegenerative diseases, for example Huntington disease (HD), Kennedy disease (spinal and bulbar muscular atrophy; SBMA), dentatorubral-pallidoluysian atrophy (DRPLA), Alzheimer's disease (AD) and 6 spinocerebellar ataxias (SCAs) and cancers. See, e.g., Iyer et al. Oncogene (2004) 23, 4225-4231; and Valor et al. Curr Pharm Des. 2013 August; 19(28): 5051-5064, the entire contents of each of which are incorporated herein by reference.

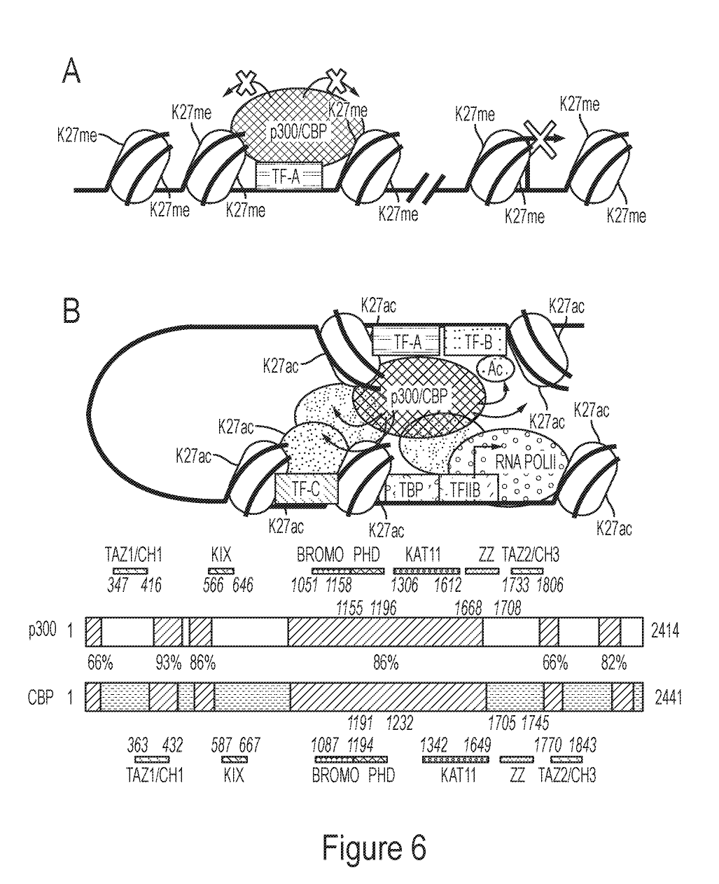

Discrete functions have been attributed to individual domains of the CREBBP protein. See, e.g., Liu et al. Nature 451, 846-850; Vo et. al. J Biol Chem. 2001 Apr. 27; 276(17):13505-8; Kalkhoven et al. Biochemical Pharmacology 68 (2004) pg. 1145-1155; and Farria et al. Oncogene (2015) 34, 4901-4913, the entire contents of each of which are incorporated herein by reference. For example, kinase inducible domain interacting (KIX), bromo-, and histone acetyl transferase (HAT) domains have been defined in the CREBBP protein. Table 1 presents a polypeptide sequence of CREBBP protein (GenBank Accession Number AAC51331.2; SEQ ID NO: 1). Table 1 presents a representative wild type CREBBP transcript (GenBank Accesion Number U85962; SEQ ID NO: 2). FIG. 6 is a schematic depiction of a representative wild type CREBBP/EP300 protein and representative domain localizations. The KIX domain of CREBBP protein can be found between amino acid position 587-667 of SEQ ID NO: 1. The bromodomain of CREBBP protein can be found between amino acids 1087-1194 of SEQ ID NO: 1. The HAT domain of CREBBP protein can be found between amino acids 1323-1700 of SEQ ID NO:1. Table 1 also provides exemplary sequences for EP300 (GenBank Accesion Number NM_001420; SEQ ID NO: 3; and GenBank Accesion Number NP_001429; SEQ ID NO: 4).

TABLE 1
GenBank Accession Number AAC51331.2 SEQ ID NO: 1
MAENLLDGPPNPKRAKLSSPGFSANDSTDFGSLFDLENDLPDELIPNGGELGLLNSGNLVPDAASKHKQL
SELLRGGSGSSINPGIGNVSASSPVQQGLGGQAQGQPNSANMASLSAMGKSPLSQGDSSAPSLPKQAAST
SGPTPAASQALNPQAQKQVGLATSSPATSQTGPGICMNANFNQTHPGLLNSNSGHSLINQASQGQAQVMN
GSLGAAGRGRGAGMPYPTPAMQGASSSVLAETLTQVSPQMTGHAGLNTAQAGGMAKMGITGNTSPFGQPF
SQAGGQPMGATGVNPQLASKQSMVNSLPTFPTDIKNTSVTNVPNMSQMQTSVGIVPTQAIATGPTADPEK
RKLIQQQLVLLLHAHKCQRREQANGEVRACSLPHCRTMKNVLNHMTHCQAGKACQVAHCASSRQIISHWK
NCTRHDCPVCLPLKNASDKRNQQTILGSPASGIQNTIGSVGTGQQNATSLSNPNPIDPSSMQRAYAALGL
PYMNQPQTQLQPQVPGQQPAQPQTHQQMRTLNPLGNNPMNIPAGGITTDQQPPNLISESALPTSLGATNP
LMNDGSNSGNIGTLSTIPTAAPPSSTGVRKGWHEHVTQDLRSHLVHKLVQAIFPTPDPAALKDRRMENLV
AYAKKVEGDMYESANSRDEYYHLLAEKIYKIQKELEEKRRSRLHKQGILGNQPALPAPGAQPPVIPQAQP
VRPPNGPLSLPVNRMQVSQGMNSFNPMSLGNVQLPQAPMGPRAASPMNHSVQMNSMGSVPGMAISPSRMP
QPPNMMGAHTNNMMAQAPAQSQFLPQNQFPSSSGAMSVGMGQPPAQTGVSQGQVPGAALPNPLNMLGPQA
SQLPCPPVTQSPLHPTPPPASTAAGMPSLQHTTPPGMTPPQPAAPTQPSTPVSSSGQTPTPTPGSVPSAT
QTQSTPTVQAAAQAQVTPQPQTPVQPPSVATPQSSQQQPTPVHAQPPGTPLSQAAASIDNRVPTPSSVAS
AETNSQQPGPDVPVLEMKTETQAEDTEPDPGESKGEPRSEMMEEDLQGASQVKEETDIAEQKSEPMEVDE
KKPEVKVEVKEEEESSSNGTASQSTSPSQPRKKIFKPEELRQALMPTLEALYRQDPESLPFRQPVDPQLL
GIPDYFDIVKNPMDLSTIKRKLDTGQYQEPWQYVDDVWLMFNNAWLYNRKTSRVYKFCSKLAEVFEQEID
PVMQSLGYCCGRKYEFSPQTLCCYGKQLCTIPRDAAYYSYQNRYHFCEKCFTEIQGENVTLGDDPSQPQT
TISKDQFEKKKNDTLDPEPFVDCKECGRKMHQICVLHYDIIWPSGFVCDNCLKKTGRPRKENKFSAKRLQ
TTRLGNHLEDRVNKFLRRQNHPEAGEVFVRVVASSDKTVEVKPGMKSRFVDSGEMSESFPYRTKALFAFE
EIDGVDVCFFGMHVQEYGSDCPPPNTRRVYISYLDSIHFFRPRCLRTAVYHEILIGYLEYVKKLGYVTGH
IWACPPSEGDDYIFHCHPPDQKIPKPKRLQEWYKKMLDKAFAERIIHDYKDIFKQATEDRLTSAKELPYF
EGDFWPNVLEESIKELEQEEEERKKEESTAASETTEGSQGDSKNAKKKNNKKTNKNKSSISRANKKKPSM
PNVSNDLSQKLYATMEKHKEVFFVIHLHAGPVINTLPPIVDPDPLLSCDLMDGRDAFLTLARDKHWEFSS
LRRSKWSTLCMLVELHTQGQDRFVYTCNECKHHVETRWHCTVCEDYDLCINCYNTKSHAHKMVKWGLGLD
DEGSSQGEPQSKSPQESRRLSIQRCIQSLVHACQCRNANCSLPSCQKMKRVVQHTKGCKRKTNGGCPVCK
QLIALCCYHAKHCQENKCPVPFCLNIKHKLRQQQIQHRLQQAQLMRRRMATMNTRNVPQQSLPSPTSAPP
GTPTQQPSTPQTPQPPAQPQPSPVSMSPAGFPSVARTQPPTTVSTGKPTSQVPAPPPPAQPPPAAVEAAR
QIEREAQQQQHLYRVNINNSMPPGRTGMGTPGSQMAPVSLNVPRPNQVSGPVMPSMPPGQWQQAPLPQQQ
PMPGLPRPVISMQAQAAVAGPRMPSVQPPRSISPSALQDLLRTLKSPSSPQQQQQVLNILKSNPQLMAAF
IKQRTAKYVANQPGMQPQPGLQSQPGMQPQPGMHQQPSLQNLNAMQAGVPRPGVPPQQQAMGGLNPQGQA
LNIMNPGHNPNMASMNPQYREMLRRQLLQQQQQQQQQQQQQQQQQQGSAGMAGGMAGHGQFQQPQGPGGY
PPAMQQQQRMQQHLPLQGSSMGQMAAQMGQLGQMGQPGLGADSTPNIQQALQQRILQQQQMKQQIGSPGQ
PNPMSPQQHMLSGQPQASHLPGQQIATSLSNQVRSPAPVQSPRPQSQPPHSSPSPRIQPQPSPHHVSPQT
GSPHPGLAVTMASSIDQGHLGNPEQSAMLPQLNTPSRSALSSELSLVGDTTGDTLEKFVEGL
GenBank Accesion Number U85962 (SEQ ID NO: 2)
TCCGAATTCCTTTTTTTTAATTGAGGAATCAACAGCCGCCATCTTGTCGCGGACCCGACCGGGGCTTCGA
GCGCGATCTACTCGGCCCCGCCGGTCCCGGGCCCCACAACCGCCCGCGCACCCCGCTCCGCCCGGCCGGC
CCGCTCCGCCCGGCCCTCGGCGCCCGCCCCGGCGGCCCCGCTCGCCTCTCGGCTCGGCCTCCCGGAGCCC
GGCGGCGGCGGCGGCGGCAGCGGCGGCGGCGGCGGCGGAACGGGGGGTGGGGGGGCCGCGGCGGCGGCGG
CGACCCCGCTCGGCGCATTGTTTTTCCTCACGGCGGCGGCGGCGGCGGGCCGCGGGCCGGGAGCGGAGCC
CGGAGCCCCCTCGTCGTCGGGCCGCGAGCGAATTCATTAAGTGGGGCGCGGGGGGGGAGCGAGGCGGCGG
CGGCGGCGGCACCATGTTCTCGGGGACTGCCTGAGCCGCCCGGCCGGGCGCCGTCGCTGCCAGCCGGGCC
CGGGGGGGCGGCCGGGCCGCCGGGGCGCCCCCACCGCGGAGTGTCGCGCTCGGGAGGCGGGCAGGGGATG
AGGGGGCCGCGGCCGGCGGCGGCGGCGGCGGCCGGGGGCGGGCGGTGAGCGCTGCGGGGCGCTGTTGCTG
TGGCTGAGATTTGGCCGCCGCCTCCCCCACCCGGCCTGCGCCCTCCCTCTCCCTCGGCGCCCGCCCGCGC
CGCTCGCGGCGCCCGCGCTCGCTCCTCTCCCTCGCAGCCGGCAGGGCCCCCGACCCCCGTCCGGGCCCTC
GCCGGCCCGGCCGCCCGTGCCCGGGGCTGTTTTCGCGAGCAGGTGAAAATGGCTGAGAACTTGCTGGACG
GACCGCCCAACCCCAAAAGAGCCAAACTCAGCTCGCCCGGTTTCTCGGCGAATGACAGCACAGATTTTGG
ATCATTGTTTGACTTGGAAAATGATCTTCCTGATGAGCTGATACCCAATGGAGGAGAATTAGGCCTTTTA
AACAGTGGGAACCTTGTTCCAGATGCTGCTTCCAAACATAAACAACTGTCGGAGCTTCTACGAGGAGGCA
GCGGCTCTAGTATCAACCCAGGAATAGGAAATGTGAGCGCCAGCAGCCCCGTGCAGCAGGGCCTGGGTGG
CCAGGCTCAAGGGCAGCCGAACAGTGCTAACATGGCCAGCCTCAGTGCCATGGGCAAGAGCCCTCTGAGC
CAGGGAGATTCTTCAGCCCCCAGCCTGCCTAAACAGGCAGCCAGCACCTCTGGGCCCACCCCCGCTGCCT
CCCAAGCACTGAATCCGCAAGCACAAAAGCAAGTGGGGCTGGCGACTAGCAGCCCTGCCACGTCACAGAC
TGGACCTGGTATCTGCATGAATGCTAACTTTAACCAGACCCACCCAGGCCTCCTCAATAGTAACTCTGGC
CATAGCTTAATTAATCAGGCTTCACAAGGGCAGGCGCAAGTCATGAATGGATCTCTTGGGGCTGCTGGCA
GAGGAAGGGGAGCTGGAATGCCGTACCCTACTCCAGCCATGCAGGGCGCCTCGAGCAGCGTGCTGGCTGA
GACCCTAACGCAGGTTTCCCCGCAAATGACTGGTCACGCGGGACTGAACACCGCACAGGCAGGAGGCATG
GCCAAGATGGGAATAACTGGGAACACAAGTCCATTTGGACAGCCCTTTAGTCAAGCTGGAGGGCAGCCAA
TGGGAGCCACTGGAGTGAACCCCCAGTTAGCCAGCAAACAGAGCATGGTCAACAGTTTGCCCACCTTCCC
TACAGATATCAAGAATACTTCAGTCACCAACGTGCCAAATATGTCTCAGATGCAAACATCAGTGGGAATT
GTACCCACACAAGCAATTGCAACAGGCCCCACTGCAGATCCTGAAAAACGCAAACTGATACAGCAGCAGC
TGGTTCTACTGCTTCATGCTCATAAGTGTCAGAGACGAGAGCAAGCAAACGGAGAGGTTCGGGCCTGCTC
GCTCCCGCATTGTCGAACCATGAAAAACGTTTTGAATCACATGACGCATTGTCAGGCTGGGAAAGCCTGC
CAAGTTGCCCATTGTGCATCTTCACGACAAATCATCTCTCATTGGAAGAACTGCACACGACATGACTGTC
CTGTTTGCCTCCCTTTGAAAAATGCCAGTGACAAGCGAAACCAACAAACCATCCTGGGGTCTCCAGCTAG
TGGAATTCAAAACACAATTGGTTCTGTTGGCACAGGGCAACAGAATGCCACTTCTTTAAGTAACCCAAAT
CCCATAGACCCCAGCTCCATGCAGCGAGCCTATGCTGCTCTCGGACTCCCCTACATGAACCAGCCCCAGA
CGCAGCTGCAGCCTCAGGTTCCTGGCCAGCAACCAGCACAGCCTCAAACCCACCAGCAGATGAGGACTCT
CAACCCCCTGGGAAATAATCCAATGAACATTCCAGCAGGAGGAATAACAACAGATCAGCAGCCCCCAAAC
TTGATTTCAGAATCAGCTCTTCCGACTTCCCTGGGGGCCACAAACCCACTGATGAACGATGGCTCCAACT
CTGGTAACATTGGAACCCTCAGCACTATACCAACAGCAGCTCCTCCTTCTAGCACCGGTGTAAGGAAAGG
CTGGCACGAACATGTCACTCAGGACCTGCGGAGCCATCTAGTGCATAAACTCGTCCAAGCCATCTTCCCA
ACACCTGATCCCGCAGCTCTAAAGGATCGCCGCATGGAAAACCTGGTAGCCTATGCTAAGAAAGTGGAAG
GGGACATGTACGAGTCTGCCAACAGCAGGGATGAATATTATCACTTATTAGCAGAGAAAATCTACAAGAT
ACAAAAAGAACTAGAAGAAAAACGGAGGTCGCGTTTACATAAACAAGGCATCTTGGGGAACCAGCCAGCC
TTACCAGCCCCGGGGGCTCAGCCCCCTGTGATTCCACAGGCACAACCTGTGAGACCTCCAAATGGACCCC
TGTCCCTGCCAGTGAATCGCATGCAAGTTTCTCAAGGGATGAATTCATTTAACCCCATGTCCTTGGGGAA
CGTCCAGTTGCCACAAGCACCCATGGGACCTCGTGCAGCCTCCCCAATGAACCACTCTGTCCAGATGAAC
AGCATGGGCTCAGTGCCAGGGATGGCCATTTCTCCTTCCCGAATGCCTCAGCCTCCGAACATGATGGGTG
CACACACCAACAACATGATGGCCCAGGCGCCCGCTCAGAGCCAGTTTCTGCCACAGAACCAGTTCCCGTC
ATCCAGCGGGGCGATGAGTGTGGGCATGGGGCAGCCGCCAGCCCAAACAGGCGTGTCACAGGGACAGGTG
CCTGGTGCTGCTCTTCCTAACCCTCTCAACATGCTGGGGCCTCAGGCCAGCCAGCTACCTTGCCCTCCAG
TGACACAGTCACCACTGCACCCAACACCGCCTCCTGCTTCCACGGCTGCTGGCATGCCATCTCTCCAGCA
CACGACACCACCTGGGATGACTCCTCCCCAGCCAGCAGCTCCCACTCAGCCATCAACTCCTGTGTCGTCT
TCCGGGCAGACTCCCACCCCGACTCCTGGCTCAGTGCCCAGTGCTACCCAAACCCAGAGCACCCCTACAG
TCCAGGCAGCAGCCCAGGCCCAGGTGACCCCGCAGCCTCAAACCCCAGTTCAGCCCCCGTCTGTGGCTAC
CCCTCAGTCATCGCAGCAACAGCCGACGCCTGTGCACGCCCAGCCTCCTGGCACACCGCTTTCCCAGGCA
GCAGCCAGCATTGATAACAGAGTCCCTACCCCCTCCTCGGTGGCCAGCGCAGAAACCAATTCCCAGCAGC
CAGGACCTGACGTACCTGTGCTGGAAATGAAGACGGAGACCCAAGCAGAGGACACTGAGCCCGATCCTGG
TGAATCCAAAGGGGAGCCCAGGTCTGAGATGATGGAGGAGGATTTGCAAGGAGCTTCCCAAGTTAAAGAA
GAAACAGACATAGCAGAGCAGAAATCAGAACCAATGGAAGTGGATGAAAAGAAACCTGAAGTGAAAGTAG
AAGTTAAAGAGGAAGAAGAGAGTAGCAGTAACGGCACAGCCTCTCAGTCAACATCTCCTTCGCAGCCGCG
CAAAAAAATCTTTAAACCAGAGGAGTTACGCCAGGCCCTCATGCCAACCCTAGAAGCACTGTATCGACAG
GACCCAGAGTCATTACCTTTCCGGCAGCCTGTAGATCCCCAGCTCCTCGGAATTCCAGACTATTTTGACA
TCGTAAAGAATCCCATGGACCTCTCCACCATCAAGCGGAAGCTGGACACAGGGCAATACCAAGAGCCCTG
GCAGTACGTGGACGACGTCTGGCTCATGTTCAACAATGCCTGGCTCTATAATCGCAAGACATCCCGAGTC
TATAAGTTTTGCAGTAAGCTTGCAGAGGTCTTTGAGCAGGAAATTGACCCTGTCATGCAGTCCCTTGGAT
ATTGCTGTGGACGCAAGTATGAGTTTTCCCCACAGACTTTGTGCTGCTATGGGAAGCAGCTGTGTACCAT
TCCTCGCGATGCTGCCTACTACAGCTATCAGAATAGGTATCATTTCTGTGAGAAGTGTTTCACAGAGATC
CAGGGCGAGAATGTGACCCTGGGTGACGACCCTTCACAGCCCCAGACGACAATTTCAAAGGATCAGTTTG
AAAAGAAGAAAAATGATACCTTAGACCCCGAACCTTTCGTTGATTGCAAGGAGTGTGGCCGGAAGATGCA
TCAGATTTGCGTTCTGCACTATGACATCATTTGGCCTTCAGGTTTTGTGTGCGACAACTGCTTGAAGAAA
ACTGGCAGACCTCGAAAAGAAAACAAATTCAGTGCTAAGAGGCTGCAGACCACAAGACTGGGAAACCACT
TGGAAGACCGAGTGAACAAATTTTTGCGGCGCCAGAATCACCCTGAAGCCGGGGAGGTTTTTGTCCGAGT
GGTGGCCAGCTCAGACAAGACGGTGGAGGTCAAGCCCGGGATGAAGTCACGGTTTGTGGATTCTGGGGAA
ATGTCTGAATCTTTCCCATATCGAACCAAAGCTCTGTTTGCTTTTGAGGAAATTGACGGCGTGGATGTCT
GCTTTTTTGGAATGCACGTCCAAGAATACGGCTCTGATTGCCCCCCTCCAAACACGAGGCGTGTGTACAT
TTCTTATCTGGATAGTATTCATTTCTTCCGGCCACGTTGCCTCCGCACAGCCGTTTACCATGAGATCCTT
ATTGGATATTTAGAGTATGTGAAGAAATTAGGGTATGTGACAGGGCACATCTGGGCCTGTCCTCCAAGTG
AAGGAGATGATTACATCTTCCATTGCCACCCACCTGATCAAAAAATACCCAAGCCAAAACGACTGCAGGA
GTGGTACAAAAAGATGCTGGACAAGGCGTTTGCAGAGCGGATCATCCATGACTACAAGGATATTTTCAAA
CAAGCAACTGAAGACAGGCTCACCAGTGCCAAGGAACTGCCCTATTTTGAAGGTGATTTCTGGCCCAATG
TGTTAGAAGAGAGCATTAAGGAACTAGAACAAGAAGAAGAGGAGAGGAAAAAGGAAGAGAGCACTGCAGC
CAGTGAAACCACTGAGGGCAGTCAGGGCGACAGCAAGAATGCCAAGAAGAAGAACAACAAGAAAACCAAC
AAGAACAAAAGCAGCATCAGCCGCGCCAACAAGAAGAAGCCCAGCATGCCCAACGTGTCCAATGACCTGT
CCCAGAAGCTGTATGCCACCATGGAGAAGCACAAGGAGGTCTTCTTCGTGATCCACCTGCACGCTGGGCC
TGTCATCAACACCCTGCCCCCCATCGTCGACCCCGACCCCCTGCTCAGCTGTGACCTCATGGATGGGCGC
GACGCCTTCCTCACCCTCGCCAGAGACAAGCACTGGGAGTTCTCCTCCTTGCGCCGCTCCAAGTGGTCCA
CGCTCTGCATGCTGGTGGAGCTGCACACCCAGGGCCAGGACCGCTTTGTCTACACCTGCAACGAGTGCAA
GCACCACGTGGAGACGCGCTGGCACTGCACTGTGTGCGAGGACTACGACCTCTGCATCAACTGCTATAAC
ACGAAGAGCCATGCCCATAAGATGGTGAAGTGGGGGCTGGGCCTGGATGACGAGGGCAGCAGCCAGGGCG
AGCCACAGTCAAAGAGCCCCCAGGAGTCACGCCGGCTGAGCATCCAGCGCTGCATCCAGTCGCTGGTGCA
CGCGTGCCAGTGCCGCAACGCCAACTGCTCGCTGCCATCCTGCCAGAAGATGAAGCGGGTGGTGCAGCAC
ACCAAGGGCTGCAAACGCAAGACCAACGGGGGCTGCCCGGTGTGCAAGCAGCTCATCGCCCTCTGCTGCT
ACCACGCCAAGCACTGCCAAGAAAACAAATGCCCCGTGCCCTTCTGCCTCAACATCAAACACAAGCTCCG
CCAGCAGCAGATCCAGCACCGCCTGCAGCAGGCCCAGCTCATGCGCCGGCGGATGGCCACCATGAACACC
CGCAACGTGCCTCAGCAGAGTCTGCCTTCTCCTACCTCAGCACCGCCCGGGACCCCCACACAGCAGCCCA
GCACACCCCAGACGCCGCAGCCCCCTGCCCAGCCCCAACCCTCACCCGTGAGCATGTCACCAGCTGGCTT
CCCCAGCGTGGCCCGGACTCAGCCCCCCACCACGGTGTCCACAGGGAAGCCTACCAGCCAGGTGCCGGCC
CCCCCACCCCCGGCCCAGCCCCCTCCTGCAGCGGTGGAAGCGGCTCGGCAGATCGAGCGTGAGGCCCAGC
AGCAGCAGCACCTGTACCGGGTGAACATCAACAACAGCATGCCCCCAGGACGCACGGGCATGGGGACCCC
GGGGAGCCAGATGGCCCCCGTGAGCCTGAATGTGCCCCGACCCAACCAGGTGAGCGGGCCCGTCATGCCC
AGCATGCCTCCCGGGCAGTGGCAGCAGGCGCCCCTTCCCCAGCAGCAGCCCATGCCAGGCTTGCCCAGGC
CTGTGATATCCATGCAGGCCCAGGCGGCCGTGGCTGGGCCCCGGATGCCCAGCGTGCAGCCACCCAGGAG
CATCTCACCCAGCGCTCTGCAAGACCTGCTGCGGACCCTGAAGTCGCCCAGCTCCCCTCAGCAGCAACAG
CAGGTGCTGAACATTCTCAAATCAAACCCGCAGCTAATGGCAGCTTTCATCAAACAGCGCACAGCCAAGT
ACGTGGCCAATCAGCCCGGCATGCAGCCCCAGCCTGGCCTCCAGTCCCAGCCCGGCATGCAACCCCAGCC
TGGCATGCACCAGCAGCCCAGCCTGCAGAACCTGAATGCCATGCAGGCTGGCGTGCCGCGGCCCGGTGTG
CCTCCACAGCAGCAGGCGATGGGAGGCCTGAACCCCCAGGGCCAGGCCTTGAACATCATGAACCCAGGAC
ACAACCCCAACATGGCGAGTATGAATCCACAGTACCGAGAAATGTTACGGAGGCAGCTGCTGCAGCAGCA
GCAGCAACAGCAGCAGCAACAACAGCAGCAACAGCAGCAGCAGCAAGGGAGTGCCGGCATGGCTGGGGGC
ATGGCGGGGCACGGCCAGTTCCAGCAGCCTCAAGGACCCGGAGGCTACCCACCGGCCATGCAGCAGCAGC
AGCGCATGCAGCAGCATCTCCCCCTCCAGGGCAGCTCCATGGGCCAGATGGCGGCTCAGATGGGACAGCT
TGGCCAGATGGGGCAGCCGGGGCTGGGGGCAGACAGCACCCCCAACATCCAGCAAGCCCTGCAGCAGCGG
ATTCTGCAGCAACAGCAGATGAAGCAGCAGATTGGGTCCCCAGGCCAGCCGAACCCCATGAGCCCCCAGC
AACACATGCTCTCAGGACAGCCACAGGCCTCGCATCTCCCTGGCCAGCAGATCGCCACGTCCCTTAGTAA
CCAGGTGCGGTCTCCAGCCCCTGTCCAGTCTCCACGGCCCCAGTCCCAGCCTCCACATTCCAGCCCGTCA
CCACGGATACAGCCCCAGCCTTCGCCACACCACGTCTCACCCCAGACTGGTTCCCCCCACCCCGGACTCG
CAGTCACCATGGCCAGCTCCATAGATCAGGGACACTTGGGGAACCCCGAACAGAGTGCAATGCTCCCCCA
GCTGAACACCCCCAGCAGGAGTGCGCTGTCCAGCGAACTGTCCCTGGTCGGGGACACCACGGGGGACACG
CTAGAGAAGTTTGTGGAGGGCTTGTAG
GenBank Accesion Number NM_001420 (SEQ ID NO: 3)
MAENVVEPGPPSAKRPKLSSPALSASASDGTDFGSLFDLEHDLPDELINSTELGLTNGGDINQLQTSLGM
VQDAASKHKQLSELLRSGSSPNLNMGVGGPGQVMASQAQQSSPGLGLINSMVKSPMTQAGLTSPNMGMGT
SGPNQGPTQSTGMMNSPVNQPAMGMNTGMNAGMNPGMLAAGNGQGIMPNQVMNGSIGAGRGRQNMQYPNP
GMGSAGNLLTEPLQQGSPQMGGQTGLRGPQPLKMGMMNNPNPYGSPYTQNPGQQIGASGLGLQIQTKTVL
SNNLSPFAMDKKAVPGGGMPNMGQQPAPQVQQPGLVTPVAQGMGSGAHTADPEKRKLIQQQLVLLLHAHK
CQRREQANGEVRQCNLPHCRTMKNVLNHMTHCQSGKSCQVAHCASSRQIISHWKNCTRHDCPVCLPLKNA
GDKRNQQPILTGAPVGLGNPSSLGVGQQSAPNLSTVSQIDPSSIERAYAALGLPYQVNQMPTQPQVQAKN
QQNQQPGQSPQGMRPMSNMSASPMGVNGGVGVQTPSLLSDSMLHSAINSQNPMMSENASVPSLGPMPTAA
QPSTTGIRKQWHEDITQDLRNHLVHKLVQAIFPTPDPAALKDRRMENLVAYARKVEGDMYESANNRAEYY
HLLAEKIYKIQKELEEKRRTRLQKQNMLPNAAGMVPVSMNPGPNMGQPQPGMTSNGPLPDPSMIRGSVPN
QMMPRITPQSGLNQFGQMSMAQPPIVPRQTPPLQHHGQLAQPGALNPPMGYGPRMQQPSNQGQFLPQTQF
PSQGMNVTNIPLAPSSGQAPVSQAQMSSSSCPVNSPIMPPGSQGSHIHCPQLPQPALHQNSPSPVPSRTP
TPHHTPPSIGAQQPPATTIPAPVPTPPAMPPGPQSQALHPPPRQTPTPPTTQLPQQVQPSLPAAPSADQP
QQQPRSQQSTAASVPTPTAPLLPPQPATPLSQPAVSIEGQVSNPPSTSSTEVNSQAIAEKQPSQEVKMEA
KMEVDQPEPADTQPEDISESKVEDCKMESTETEERSTELKTEIKEEEDQPSTSATQSSPAPGQSKKKIFK
PEELRQALMPTLEALYRQDPESLPFRQPVDPQLLGIPDYFDIVKSPMDLSTIKRKLDTGQYQEPWQYVDD
IWLMFNNAWLYNRKTSRVYKYCSKLSEVFEQEIDPVMQSLGYCCGRKLEFSPQTLCCYGKQLCTIPRDAT
YYSYQNRYHFCEKCFNEIQGESVSLGDDPSQPQTTINKEQFSKRKNDTLDPELFVECTECGRKMHQICVL
HHEIIWPAGFVCDGCLKKSARTRKENKFSAKRLPSTRLGTFLENRVNDFLRRQNHPESGEVTVRVVHASD
KTVEVKPGMKARFVDSGEMAESFPYRTKALFAFEEIDGVDLCFFGMHVQEYGSDCPPPNQRRVYISYLDS
VHFFRPKCLRTAVYHEILIGYLEYVKKLGYTTGHIWACPPSEGDDYIFHCHPPDQKIPKPKRLQEWYKKM
LDKAVSERIVHDYKDIFKQATEDRLTSAKELPYFEGDFWPNVLEESIKELEQEEEERKREENTSNESTDV
TKGDSKNAKKKNNKKTSKNKSSLSRGNKKKPGMPNVSNDLSQKLYATMEKHKEVFFVIRLIAGPAANSLP
PIVDPDPLIPCDLMDGRDAFLTLARDKHLEFSSLRRAQWSTMCMLVELHTQSQDRFVYTCNECKHHVETR
WHCTVCEDYDLCITCYNTKNHDHKMEKLGLGLDDESNNQQAAATQSPGDSRRLSIQRCIQSLVHACQCRN
ANCSLPSCQKMKRVVQHTKGCKRKTNGGCPICKQLIALCCYHAKHCQENKCPVPFCLNIKQKLRQQQLQH
RLQQAQMLRRRMASMQRTGVVGQQQGLPSPTPATPTTPTGQQPTTPQTPQPTSQPQPTPPNSMPPYLPRT
QAAGPVSQGKAAGQVTPPTPPQTAQPPLPGPPPAAVEMAMQIQRAAETQRQMAHVQIFQRPIQHQMPPMT
PMAPMGMNPPPMTRGPSGHLEPGMGPTGMQQQPPWSQGGLPQPQQLQSGMPRPAMMSVAQHGQPLNMAPQ
PGLGQVGISPLKPGTVSQQALQNLLRTLRSPSSPLQQQQVLSILHANPQLLAAFIKQRAAKYANSNPQPI
PGQPGMPQGQPGLQPPTMPGQQGVHSNPAMQNMNPMQAGVQRAGLPQQQPQQQLQPPMGGMSPQAQQMNM
NHNTMPSQFRDILRRQQMMQQQQQQGAGPGIGPGMANHNQFQQPQGVGYPPQQQQRMQHHMQQMQQGNMG
QIGQLPQALGAEAGASLQAYQQRLLQQQMGSPVQPNPMSPQQHMLPNQAQSPHLQGQQIPNSLSNQVRSP
QPVPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
GenBank Accesion Number NM_001429 (SEQ ID NO: 4)
GCCGAGGAGGAAGAGGTTGATGGCGGCGGCGGAGCTCCGAGAGACCTCGGCTGGGCAGGGGCCGGCCGTG
GCGGGCCGGGGACTGCGCCTCTAGAGCCGCGAGTTCTCGGGAATTCGCCGCAGCGGACGCGCTCGGCGAA
TTTGTGCTCTTGTGCCCTCCTCCGGGCTTGGGCCCAGGCCCGGCCCCTCGCACTTGCCCTTACCTTTTCT
ATCGAGTCCGCATCCCTCTCCAGCCACTGCGACCCGGCGAAGAGAAAAAGGAACTTCCCCCACCCCCTCG
GGTGCCGTCGGAGCCCCCCAGCCCACCCCTGGGTGCGGCGCGGGGACCCCGGGCCGAAGAAGAGATTTCC
TGAGGATTCTGGTTTTCCTCGCTTGTATCTCCGAAAGAATTAAAAATGGCCGAGAATGTGGTGGAACCGG
GGCCGCCTTCAGCCAAGCGGCCTAAACTCTCATCTCCGGCCCTCTCGGCGTCCGCCAGCGATGGCACAGA
TTTTGGCTCTCTATTTGACTTGGAGCACGACTTACCAGATGAATTAATCAACTCTACAGAATTGGGACTA
ACCAATGGTGGTGATATTAATCAGCTTCAGACAAGTCTTGGCATGGTACAAGATGCAGCTTCTAAACATA
AACAGCTGTCAGAATTGCTGCGATCTGGTAGTTCCCCTAACCTCAATATGGGAGTTGGTGGCCCAGGTCA
AGTCATGGCCAGCCAGGCCCAACAGAGCAGTCCTGGATTAGGTTTGATAAATAGCATGGTCAAAAGCCCA
ATGACACAGGCAGGCTTGACTTCTCCCAACATGGGGATGGGCACTAGTGGACCAAATCAGGGTCCTACGC
AGTCAACAGGTATGATGAACAGTCCAGTAAATCAGCCTGCCATGGGAATGAACACAGGGATGAATGCGGG
CATGAATCCTGGAATGTTGGCTGCAGGCAATGGACAAGGGATAATGCCTAATCAAGTCATGAACGGTTCA
ATTGGAGCAGGCCGAGGGCGACAGAATATGCAGTACCCAAACCCAGGCATGGGAAGTGCTGGCAACTTAC
TGACTGAGCCTCTTCAGCAGGGCTCTCCCCAGATGGGAGGACAAACAGGATTGAGAGGCCCCCAGCCTCT
TAAGATGGGAATGATGAACAACCCCAATCCTTATGGTTCACCATATACTCAGAATCCTGGACAGCAGATT
GGAGCCAGTGGCCTTGGTCTCCAGATTCAGACAAAAACTGTACTATCAAATAACTTATCTCCATTTGCTA
TGGACAAAAAGGCAGTTCCTGGTGGAGGAATGCCCAACATGGGTCAACAGCCAGCCCCGCAGGTCCAGCA
GCCAGGCCTGGTGACTCCAGTTGCCCAAGGGATGGGTTCTGGAGCACATACAGCTGATCCAGAGAAGCGC
AAGCTCATCCAGCAGCAGCTTGTTCTCCTTTTGCATGCTCACAAGTGCCAGCGCCGGGAACAGGCCAATG
GGGAAGTGAGGCAGTGCAACCTTCCCCACTGTCGCACAATGAAGAATGTCCTAAACCACATGACACACTG
CCAGTCAGGCAAGTCTTGCCAAGTGGCACACTGTGCATCTTCTCGACAAATCATTTCACACTGGAAGAAT
TGTACAAGACATGATTGTCCTGTGTGTCTCCCCCTCAAAAATGCTGGTGATAAGAGAAATCAACAGCCAA
TTTTGACTGGAGCACCCGTTGGACTTGGAAATCCTAGCTCTCTAGGGGTGGGTCAACAGTCTGCCCCCAA
CCTAAGCACTGTTAGTCAGATTGATCCCAGCTCCATAGAAAGAGCCTATGCAGCTCTTGGACTACCCTAT
CAAGTAAATCAGATGCCGACACAACCCCAGGTGCAAGCAAAGAACCAGCAGAATCAGCAGCCTGGGCAGT
CTCCCCAAGGCATGCGGCCCATGAGCAACATGAGTGCTAGTCCTATGGGAGTAAATGGAGGTGTAGGAGT
TCAAACGCCGAGTCTTCTTTCTGACTCAATGTTGCATTCAGCCATAAATTCTCAAAACCCAATGATGAGT
GAAAATGCCAGTGTGCCCTCCCTGGGTCCTATGCCAACAGCAGCTCAACCATCCACTACTGGAATTCGGA
AACAGTGGCACGAAGATATTACTCAGGATCTTCGAAATCATCTTGTTCACAAACTCGTCCAAGCCATATT
TCCTACGCCGGATCCTGCTGCTTTAAAAGACAGACGGATGGAAAACCTAGTTGCATATGCTCGGAAAGTT
GAAGGGGACATGTATGAATCTGCAAACAATCGAGCGGAATACTACCACCTTCTAGCTGAGAAAATCTATA
AGATCCAGAAAGAACTAGAAGAAAAACGAAGGACCAGACTACAGAAGCAGAACATGCTACCAAATGCTGC
AGGCATGGTTCCAGTTTCCATGAATCCAGGGCCTAACATGGGACAGCCGCAACCAGGAATGACTTCTAAT
GGCCCTCTACCTGACCCAAGTATGATCCGTGGCAGTGTGCCAAACCAGATGATGCCTCGAATAACTCCAC
AATCTGGTTTGAATCAATTTGGCCAGATGAGCATGGCCCAGCCCCCTATTGTACCCCGGCAAACCCCTCC
TCTTCAGCACCATGGACAGTTGGCTCAACCTGGAGCTCTCAACCCGCCTATGGGCTATGGGCCTCGTATG
CAACAGCCTTCCAACCAGGGCCAGTTCCTTCCTCAGACTCAGTTCCCATCACAGGGAATGAATGTAACAA
ATATCCCTTTGGCTCCGTCCAGCGGTCAAGCTCCAGTGTCTCAAGCACAAATGTCTAGTTCTTCCTGCCC
GGTGAACTCTCCTATAATGCCTCCAGGGTCTCAGGGGAGCCACATTCACTGTCCCCAGCTTCCTCAACCA
GCTCTTCATCAGAATTCACCCTCGCCTGTACCTAGTCGTACCCCCACCCCTCACCATACTCCCCCAAGCA
TAGGGGCTCAGCAGCCACCAGCAACAACAATTCCAGCCCCTGTTCCTACACCTCCTGCCATGCCACCTGG
GCCACAGTCCCAGGCTCTACATCCCCCTCCAAGGCAGACACCTACACCACCAACAACACAACTTCCCCAA
CAAGTGCAGCCTTCACTTCCTGCTGCACCTTCTGCTGACCAGCCCCAGCAGCAGCCTCGCTCACAGCAGA
GCACAGCAGCGTCTGTTCCTACCCCAACAGCACCGCTGCTTCCTCCGCAGCCTGCAACTCCACTTTCCCA
GCCAGCTGTAAGCATTGAAGGACAGGTATCAAATCCTCCATCTACTAGTAGCACAGAAGTGAATTCTCAG
GCCATTGCTGAGAAGCAGCCTTCCCAGGAAGTGAAGATGGAGGCCAAAATGGAAGTGGATCAACCAGAAC
CAGCAGATACTCAGCCGGAGGATATTTCAGAGTCTAAAGTGGAAGACTGTAAAATGGAATCTACCGAAAC
AGAAGAGAGAAGCACTGAGTTAAAAACTGAAATAAAAGAGGAGGAAGACCAGCCAAGTACTTCAGCTACC
CAGTCATCTCCGGCTCCAGGACAGTCAAAGAAAAAGATTTTCAAACCAGAAGAACTACGACAGGCACTGA
TGCCAACTTTGGAGGCACTTTACCGTCAGGATCCAGAATCCCTTCCCTTTCGTCAACCTGTGGACCCTCA
GCTTTTAGGAATCCCTGATTACTTTGATATTGTGAAGAGCCCCATGGATCTTTCTACCATTAAGAGGAAG
TTAGACACTGGACAGTATCAGGAGCCCTGGCAGTATGTCGATGATATTTGGCTTATGTTCAATAATGCCT
GGTTATATAACCGGAAAACATCACGGGTATACAAATACTGCTCCAAGCTCTCTGAGGTCTTTGAACAAGA
AATTGACCCAGTGATGCAAAGCCTTGGATACTGTTGTGGCAGAAAGTTGGAGTTCTCTCCACAGACACTG
TGTTGCTACGGCAAACAGTTGTGCACAATACCTCGTGATGCCACTTATTACAGTTACCAGAACAGGTATC
ATTTCTGTGAGAAGTGTTTCAATGAGATCCAAGGGGAGAGCGTTTCTTTGGGGGATGACCCTTCCCAGCC
TCAAACTACAATAAATAAAGAACAATTTTCCAAGAGAAAAAATGACACACTGGATCCTGAACTGTTTGTT
GAATGTACAGAGTGCGGAAGAAAGATGCATCAGATCTGTGTCCTTCACCATGAGATCATCTGGCCTGCTG
GATTCGTCTGTGATGGCTGTTTAAAGAAAAGTGCACGAACTAGGAAAGAAAATAAGTTTTCTGCTAAAAG
GTTGCCATCTACCAGACTTGGCACCTTTCTAGAGAATCGTGTGAATGACTTTCTGAGGCGACAGAATCAC
CCTGAGTCAGGAGAGGTCACTGTTAGAGTAGTTCATGCTTCTGACAAAACCGTGGAAGTAAAACCAGGCA
TGAAAGCAAGGTTTGTGGACAGTGGAGAGATGGCAGAATCCTTTCCATACCGAACCAAAGCCCTCTTTGC
CTTTGAAGAAATTGATGGTGTTGACCTGTGCTTCTTTGGCATGCATGTTCAAGAGTATGGCTCTGACTGC
CCTCCACCCAACCAGAGGAGAGTATACATATCTTACCTCGATAGTGTTCATTTCTTCCGTCCTAAATGCT
TGAGGACTGCAGTCTATCATGAAATCCTAATTGGATATTTAGAATATGTCAAGAAATTAGGTTACACAAC
AGGGCATATTTGGGCATGTCCACCAAGTGAGGGAGATGATTATATCTTCCATTGCCATCCTCCTGACCAG
AAGATACCCAAGCCCAAGCGACTGCAGGAATGGTACAAAAAAATGCTTGACAAGGCTGTATCAGAGCGTA
TTGTCCATGACTACAAGGATATTTTTAAACAAGCTACTGAAGATAGATTAACAAGTGCAAAGGAATTGCC
TTATTTCGAGGGTGATTTCTGGCCCAATGTTCTGGAAGAAAGCATTAAGGAACTGGAACAGGAGGAAGAA
GAGAGAAAACGAGAGGAAAACACCAGCAATGAAAGCACAGATGTGACCAAGGGAGACAGCAAAAATGCTA
AAAAGAAGAATAATAAGAAAACCAGCAAAAATAAGAGCAGCCTGAGTAGGGGCAACAAGAAGAAACCCGG
GATGCCCAATGTATCTAACGACCTCTCACAGAAACTATATGCCACCATGGAGAAGCATAAAGAGGTCTTC
TTTGTGATCCGCCTCATTGCTGGCCCTGCTGCCAACTCCCTGCCTCCCATTGTTGATCCTGATCCTCTCA
TCCCCTGCGATCTGATGGATGGTCGGGATGCGTTTCTCACGCTGGCAAGGGACAAGCACCTGGAGTTCTC
TTCACTCCGAAGAGCCCAGTGGTCCACCATGTGCATGCTGGTGGAGCTGCACACGCAGAGCCAGGACCGC
TTTGTCTACACCTGCAATGAATGCAAGCACCATGTGGAGACACGCTGGCACTGTACTGTCTGTGAGGATT
ATGACTTGTGTATCACCTGCTATAACACTAAAAACCATGACCACAAAATGGAGAAACTAGGCCTTGGCTT
AGATGATGAGAGCAACAACCAGCAGGCTGCAGCCACCCAGAGCCCAGGCGATTCTCGCCGCCTGAGTATC
CAGCGCTGCATCCAGTCTCTGGTCCATGCTTGCCAGTGTCGGAATGCCAATTGCTCACTGCCATCCTGCC
AGAAGATGAAGCGGGTTGTGCAGCATACCAAGGGTTGCAAACGGAAAACCAATGGCGGGTGCCCCATCTG
CAAGCAGCTCATTGCCCTCTGCTGCTACCATGCCAAGCACTGCCAGGAGAACAAATGCCCGGTGCCGTTC
TGCCTAAACATCAAGCAGAAGCTCCGGCAGCAACAGCTGCAGCACCGACTACAGCAGGCCCAAATGCTTC
GCAGGAGGATGGCCAGCATGCAGCGGACTGGTGTGGTTGGGCAGCAACAGGGCCTCCCTTCCCCCACTCC
TGCCACTCCAACGACACCAACTGGCCAACAGCCAACCACCCCGCAGACGCCCCAGCCCACTTCTCAGCCT
CAGCCTACCCCTCCCAATAGCATGCCACCCTACTTGCCCAGGACTCAAGCTGCTGGCCCTGTGTCCCAGG
GTAAGGCAGCAGGCCAGGTGACCCCTCCAACCCCTCCTCAGACTGCTCAGCCACCCCTTCCAGGGCCCCC
ACCTGCAGCAGTGGAAATGGCAATGCAGATTCAGAGAGCAGCGGAGACGCAGCGCCAGATGGCCCACGTG
CAAATTTTTCAAAGGCCAATCCAACACCAGATGCCCCCGATGACTCCCATGGCCCCCATGGGTATGAACC
CACCTCCCATGACCAGAGGTCCCAGTGGGCATTTGGAGCCAGGGATGGGACCGACAGGGATGCAGCAACA
GCCACCCTGGAGCCAAGGAGGATTGCCTCAGCCCCAGCAACTACAGTCTGGGATGCCAAGGCCAGCCATG
ATGTCAGTGGCCCAGCATGGTCAACCTTTGAACATGGCTCCACAACCAGGATTGGGCCAGGTAGGTATCA
GCCCACTCAAACCAGGCACTGTGTCTCAACAAGCCTTACAAAACCTTTTGCGGACTCTCAGGTCTCCCAG
CTCTCCCCTGCAGCAGCAACAGGTGCTTAGTATCCTTCACGCCAACCCCCAGCTGTTGGCTGCATTCATC
AAGCAGCGGGCTGCCAAGTATGCCAACTCTAATCCACAACCCATCCCTGGGCAGCCTGGCATGCCCCAGG
GGCAGCCAGGGCTACAGCCACCTACCATGCCAGGTCAGCAGGGGGTCCACTCCAATCCAGCCATGCAGAA
CATGAATCCAATGCAGGCGGGCGTTCAGAGGGCTGGCCTGCCCCAGCAGCAACCACAGCAGCAACTCCAG
CCACCCATGGGAGGGATGAGCCCCCAGGCTCAGCAGATGAACATGAACCACAACACCATGCCTTCACAAT
TCCGAGACATCTTGAGACGACAGCAAATGATGCAACAGCAGCAGCAACAGGGAGCAGGGCCAGGAATAGG
CCCTGGAATGGCCAACCATAACCAGTTCCAGCAACCCCAAGGAGTTGGCTACCCACCACAGCAGCAGCAG
CGGATGCAGCATCACATGCAACAGATGCAACAAGGAAATATGGGACAGATAGGCCAGCTTCCCCAGGCCT
TGGGAGCAGAGGCAGGTGCCAGTCTACAGGCCTATCAGCAGCGACTCCTTCAGCAACAGATGGGGTCCCC
TGTTCAGCCCAACCCCATGAGCCCCCAGCAGCATATGCTCCCAAATCAGGCCCAGTCCCCACACCTACAA
GGCCAGCAGATCCCTAATTCTCTCTCCAATCAAGTGCGCTCTCCCCAGCCTGTCCCTTCTCCACGGCCAC
AGTCCCAGCCCCCCCACTCCAGTCCTTCCCCAAGGATGCAGCCTCAGCCTTCTCCACACCACGTTTCCCC
ACAGACAAGTTCCCCACATCCTGGACTGGTAGCTGCCCAGGCCAACCCCATGGAACAAGGGCATTTTGCC
AGCCCGGACCAGAATTCAATGCTTTCTCAGCTTGCTAGCAATCCAGGCATGGCAAACCTCCATGGTGCAA
GCGCCACGGACCTGGGACTCAGCACCGATAACTCAGACTTGAATTCAAACCTCTCACAGAGTACACTAGA
CATACACTAGAGACACCTTGTAGTATTTTGGGAGCAAAAAAATTATTTTCTCTTAACAAGACTTTTTGTA
CTGAAAACAATTTTTTTGAATCTTTCGTAGCCTAAAAGACAATTTTCCTTGGAACACATAAGAACTGTGC
AGTAGCCGTTTGTGGTTTAAAGCAAACATGCAAGATGAACCTGAGGGATGATAGAATACAAAGAATATAT
TTTTGTTATGGCTGGTTACCACCAGCCTTTCTTCCCCTTTGTGTGTGTGGTTCAAGTGTGCACTGGGAGG
AGGCTGAGGCCTGTGAAGCCAAACAATATGCTCCTGCCTTGCACCTCCAATAGGTTTTATTATTTTTTTT
AAATTAATGAACATATGTAATATTAATAGTTATTATTTACTGGTGCAGATGGTTGACATTTTTCCCTATT
TTCCTCACTTTATGGAAGAGTTAAAACATTTCTAAACCAGAGGACAAAAGGGGTTAATGTTACTTTAAAA
TTACATTCTATATATATATAAATATATATAAATATATATTAAAATACCAGTTTTTTTTCTCTGGGTGCAA
AGATGTTCATTCTTTTAAAAAATGTTTAAAAAAAAAAAAAAACTGCCTTTCTTCCCCTCAAGTCAACTTT
TGTGCTCCAGAAAATTTTCTATTCTGTAAGTCTGAGCGTAAAACTTCAAGTATTAAAATAATTTGTACAT
GTAGAGAGAAAAATGACTTTTTCAAAAATATACAGGGGCAGCTGCCAAATTGATGTATTATATATTGTGG
TTTCTGTTTCTTGAAAGAATTTTTTTCGTTATTTTTACATCTAACAAAGTAAAAAAATTAAAAAGAGGGT
AAGAAACGATTCCGGTGGGATGATTTTAACATGCAAAATGTCCCTGGGGGTTTCTTCTTTGCTTGCTTTC
TTCCTCCTTACCCTACCCCCCACTCACACACACACACACACACACACACACACACACACACACACACTTT
CTATAAAACTTGAAAATAGCAAAAACCCTCAACTGTTGTAAATCATGCAATTAAAGTTGATTACTTATAA
ATATGAACTTTGGATCACTGTATAGACTGTTAAATTTGATTTCTTATTACCTATTGTTAAATAAACTGTG
TGAGACAGACA

Discrete functions of EP300 are carried out by specific domains of the EP300 protein. See, e.g., Liu et al. Nature 451, 846-850; Vo et. al. J Biol Chem. 2001 Apr. 27; 276(17):13505-8; Kalkhoven et al. Biochemical Pharmacology 68 (2004) pg. 1145-1155; and Farria et al. Oncogene (2015) 34, 4901-4913. Table 1 presents an EP300 polypeptide sequence (GenBank Accession Number NP_001420; SEQ ID NO: 3). Table 1 presents a representative wild type EP300 transcript (GenBank Accesion Number NM_001429; SEQ ID NO:4).

The KIX domain of EP300 protein can be found between amino acid position 566-646 of SEQ ID NO: 3. The bromodomain of EP300 protein can be found between amino acid position 1051-1158 of SEQ ID NO: 3. The HAT domain of EP300 protein can be found between amino acid position 1287-1663 of SEQ ID NO: 3.

Deleterious (loss of function) mutations in the EP300 protein include, for example, substitutions, insertions, deletions, indels, missense mutations, nonsense mutations, and truncations. Exemplary loss of function mutations of EP300 include, for example, mutations at one or more of the following residues of SEQ ID NO: 3: V5, R86, K291, T329, R397, G711, P802, Q993, E1014, P1081, G1042, R1055, C1201, R1234, C1385, D1399, Y1414, A1437, Y1467, K1468, K1488, W1509, R1645, S1650, S1754, Q1874, R1950, Q2023, and Q2306.

Deleterious (loss of function) mutations in the EP300 protein include, for example, substitutions, insertions, deletions, indels, missense mutations, nonsense mutations, and truncations. Exemplary loss of function mutations of EP300 include, for example, mutations at one or more of the following residues of SEQ ID NO: 3: G30, K423, R883, T891, E1014, Q1661, and P2097.

Below is a representation of the residues listed immediately above within SEQ ID NO: 3.

| (SEQ ID NO: 3) | |
|---|---|
| 1 | 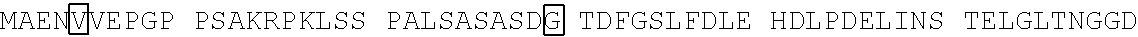 |
| 61 | 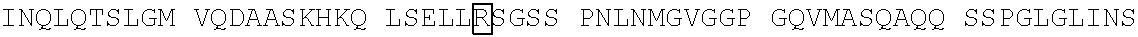 |
| 121 | MVKSPMTQAG LTSPNMGMGT SGPNQGPTQS TGMMNSPVNQ PAMGMNTGMN AGMNPGMLAA |
| 181 | GNGQGIMPNQ VMNGSIGAGR GRQNMQYPNP GMGSAGNLLT EPLQQGSPQM GGQTGLRGPQ |
| 241 | 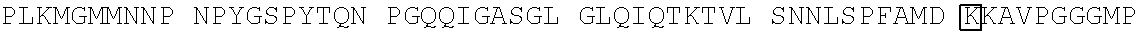 |
| 301 | 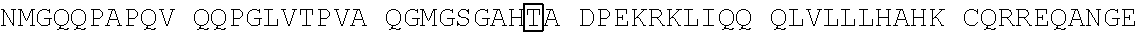 |
| 361 | 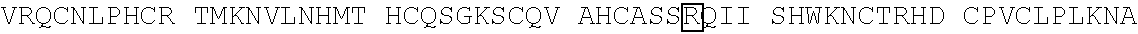 |
| 421 | 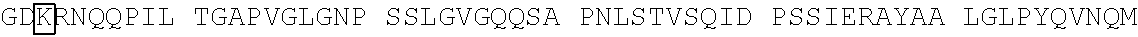 |
| 481 | PTQPQVQAKN QQNQQPGQSP QGMRPMSNMS ASPMGVNGGV GVQTPSLLSD SMLHSAINSQ |
| 541 | NPMMSENASV PSLGPMPTAA QPSTTGIRKQ WHEDITQDLR NHLVHKLVQA IFPTPDPAAL |
| 601 | KDRRMENLVA YARKVEGDMY ESANNRAEYY HLLAEKIYKI QKELEEKRRT RLQKQNMLPN |
| 661 | 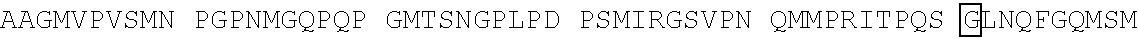 |
| 721 | AQPPIVPRQT PPLQHHGQLA QPGALNPPMG YGPRMQQPSN QGQFLPQTQF PSQGMNVTNI |
| 781 | 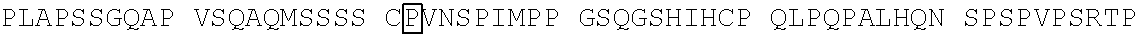 |
| 841 | 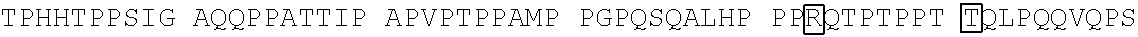 |
| 901 | LPAAPSADQP QQQPRSQQST AASVPTPTAP LLPPQPATPL SQPAVSIEGQ VSNPPSTSST |
| 961 | 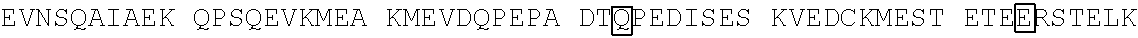 |
| 1021 | 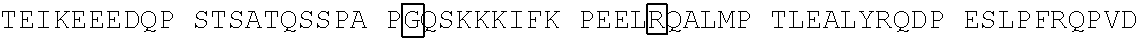 |
| 1081 | 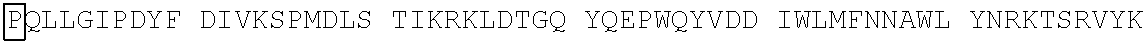 |
| 1141 | YCSKLSEVFE QEIDPVMQSL GYCCGRKLEF SPQTLCCYGK QLCTIPRDAT YYSYQNRYHF |
| 1201 | 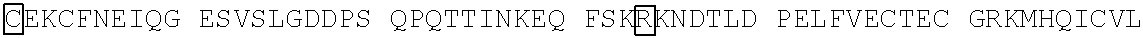 |
| 1261 | HHEIIWPAGF VCDGCLKKSA RTRKENKFSA KRLPSTRLGT FLENRVNDFL RRQNHPESGE |
| 1321 | VTVRVVHASD KTVEVKPGMK ARFVDSGEMA ESFPYRTKAL AFEEIDGVD LCFFGMHVQE |
| 1381 | 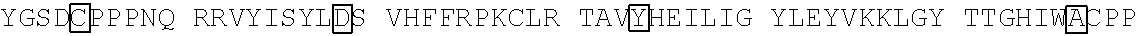 |
| 1441 | 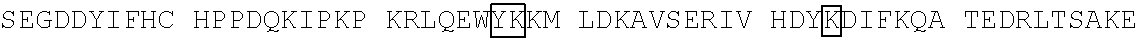 |
| 1501 | 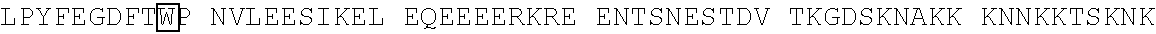 |
| 1561 | SSLSRGNKKK PGMPNVSNDL SQKLYATMEK HKEVFFVIRL IAGPAANSLP PIVDPDPLIP |
| 1621 | 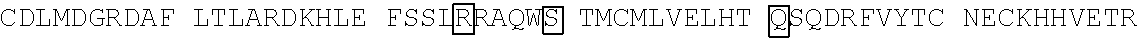 |
| 1681 | WHCTVCEDYD LCITCYNTKN HDHKMEKLGL GLDDESNNQQ AAATQSPGDS RRLSIQRCIQ |
| 1741 | 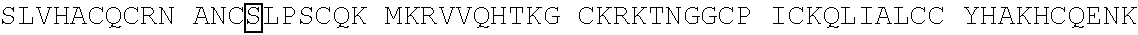 |
| 1801 | CPVPFCLNIK QKLRQQQLQH RLQQAQMLRR RMASMQRTGV VGQQQGLPSP TPATPTTPTG |
| 1861 | 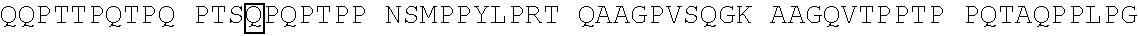 |
| 1921 | 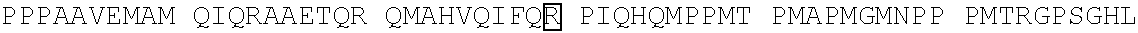 |
| 1981 | 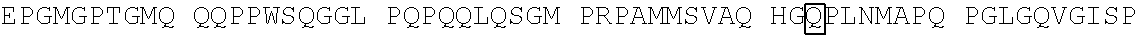 |
| 2041 | 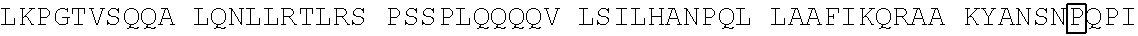 |
| 2101 | PGQPGMPQGQ PGLQPPTMPG QQGVHSNPAM QNMNPMQAGV QRAGLPQQQP QQQLQPPMGG |
| 2161 | MSPQAQQMNM NHNTMPSQFR DILRRQQMMQ QQQQQGAGPG IGPGMANHNQ FQQPQGVGYP |
| 2221 | PQQQQRMQHH MQQMQQGNMG QIGQLPQALG AEAGASLQAY QQRLLQQQMG SPVQPNPMSP |
| 2281 | 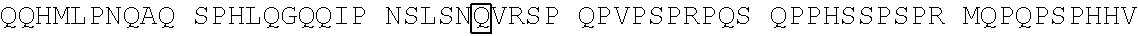 |
| 2341 | SPQTSSPHPG LVAAQANPME QGHFASPDQN SMLSQLASNP GMANLHGASA TDLGLSTDNS |
| 2401 | DLNSNLSQST LDIH |

In some embodiments, a deleterious (loss-of-function) mutation in the EP300 protein comprises a V5L, T329R, P802L, P1081S, C1201Y, C1385Y, D1399N, D1399Y, Y1414C, A1437V, W1509C, S1650Y, Q1874E, R1950G, or Q2306E substitution; a K291fs, R1234fs, K1468fs, K1488 or Y1467fs frameshift mutation; a R86*, R397*, Q993*, G1042*, R1055*, R1645*, S1754*, or Q2023* truncation; or a splice variation at G711.

In some embodiments, a deleterious (loss-of-function) mutation in the EP300 protein comprises a G30V, K423T, R883G, T891P, P2097A, or a E1014*, or Q1661* truncation.

In some embodiments, the loss of function mutation in the EP300 protein results in a truncation of the EP300 protein, e.g., by creating a premature stop codon. In some embodiments, the resulting truncated EP300 protein does not comprise a complete HAT domain, i.e., the truncation occurs within or N-terminal of the HAT domain. In some embodiments, the truncation results in the loss of at least 10%, at least 20%, at least 30%, at least 40%, at least 50%, at least 60%, at least 70%, at least 80%, at least 90% of the HAT domain of EP300. In some embodiments, the truncation results in a complete loss of the HAT domain. In some embodiments, the EP300 loss of function mutation results in a missense substitution in the EP300 protein. In some such embodiments, the missense substitution occurs within the HAT domain of EP300. In some embodiments, the EP300 loss of function mutation comprises a splice site mutation, e.g., the creation of a new splice site or the abolishment of an existing splice site in the EP300 transcript. In some such embodiments, the EP300 loss of function mutation results in a splice site mutation in a sequence encoding a part of the EP300 protein that is within the HAT domain or N-terminal of the HAT domain.

In some embodiments, a deleterious (loss-of-function) mutation in the EP300 protein, or in an encoding nucleic acid, e.g., in a genomic DNA sequence encoding an EP300 protein, comprises a mutation listed in Table 4.

TABLE 4
exemplary EP300 loss of function mutations

| Genomic DNA Change | Amino Acid Change | Consequence | Type |
|---|---|---|---|
| chr22: g.41172513_41172514insG | A1490Gfs*3 | Frame shift | Insertion |
| chr22: g.41158438delTTGC | C1177Tfs*49 | Frame shift | Deletion |
| chr22: g.41176968_41176969insG | C1753Wfs*130 | Frame shift | Insertion |
| chr22: g.41160696delG | D1217Mfs*10 | Frame shift | Deletion |
| chr22: g.41140179delA | D602Tfs*7 | Frame shift | Deletion |
| chr22: g.41172635delA | E1530Gfs*11 | Frame shift | Deletion |
| chr22: g.41177104_41177105insT | E1798Dfs*11 | Frame shift | Insertion |
| AGAAAACCAGGTCGTGAGTGGAATGTGT | | | |
| chr22: g.41164118_41164119insC | F1270Lfs*10 | Frame shift | Insertion |
| TGGCCTG | | | |
| chr22: g.41173631delGG | G1543Rfs*6 | Frame shift | Deletion |
| chr22: g.41178675delC | H2324Tfs*29 | Frame shift | Deletion |
| chr22: g.41178747_41178748insC | H2348Tfs*31 | Frame shift | Insertion |
| chr22: g.41127718delAC | H381Lfs*29 | Frame shift | Deletion |
| chr22: g.41117273delA | I61Lfs*55 | Frame shift | Deletion |
| chr22: g.41158433delCT | L1175Vfs*13 | Frameshift | Deletion |
| chr22: g.41173751_41173752insA | L1584Tfs*9 | Frameshift | Insertion |
| chr22: g.41093052_41093053insA | Li 8Tfs*21 | Frameshift | Insertion |
| chr22: g.41137789delC | L587Sfs*14 | Frameshift | Deletion |
| chr22: g.41168588_41168589insA | M1339Ifs*2 | Frameshift | Insertion |
| T | | | |
| chr22: g.41170521delA | M1470Cfs*26 | Frameshift | Deletion |
| chr22: g.41178516_41178517insT | M2269Ifs*33 | Frameshift | Insertion |
| chr22: g.41151956delA | M982Wfs*38 | Frameshift | Deletion |
| chr22: g.41162752_41162753insA | N1236Kfs*2 | Frameshift | Insertion |
| chr22: g.41162753delA | N1236Mfs*41 | Frameshift | Deletion |
| chr22: g.41173641delA | N1547Mfs*17 | Frameshift | Deletion |
| chr22: g.41129976delAATGCTGGTG | N419Ifs*9 | Frameshift | Deletion |
| chr22: g.41129973delA | N419Mfs*12 | Frameshift | Deletion |
| chr22: g.41149130delA | N779Ifs*25 | Frameshift | Deletion |
| chr22: g.41164128_41164129insG | null | Frameshift | Insertion |
| chr22: g.41177305delC | P1866Rfs*40 | Frameshift | Deletion |
| chr22: g.41177472delC | P1922Hfs*38 | Frameshift | Deletion |
| chr22: g.41137675_41137676insG | P551Cfs*51 | Frameshift | Insertion |
| T | | | |
| chr22: g.41176407delC | Q1648Sfs*61 | Frameshift | Deletion |
| chr22: g.41177969delGCAGCGG | Q2087Lfs*45 | Frameshift | Deletion |
| chr22: g.41178160delCACAGCAGC | Q2151Sfs*7 | Frameshift | Deletion |
| AACT | | | |
| chr22: g.41131606_41131607insG | Q501Rfs*12 | Frameshift | Insertion |
| chr22: g.41141203_41141204insG | Q679Afs*11 | Frameshift | Insertion |
| CTACTTACCACA | | | |
| chr22: g.41176398_41176399insC | R1645Pfs*28 | Frameshift | Insertion |
| chr22: g.41127639delC | R354Gfs*8 | Frameshift | Deletion |
| chr22: g.41149777_41149778insT | S800Ffs*38 | Frameshift | Insertion |
| chr22: g.41152302_41152303insG | T1032Sfs*57 | Frameshift | Insertion |
| chr22: g.41158476C > T | A1189V | Missense | Substitution |
| chr22: g.41117477G > T | A129S | Missense | Substitution |
| chr22: g.41170428G > A | A1437T | Missense | Substitution |
| chr22: g.41172514G > C | A1490P | Missense | Substitution |
| chr22: g.41176353C > T | A1629V | Missense | Substitution |
| chr22: g.41176945C > G | A1745G | Missense | Substitution |
| chr22: g.41177413C > T | A1901V | Missense | Substitution |
| chr22: g.41177539C > T | A1943V | Missense | Substitution |
| chr22: g.41177937G > A | A2076T | Missense | Substitution |
| chr22: g.41178124C > T | A2138V | Missense | Substitution |
| chr22: g.41178327G > T | A22065 | Missense | Substitution |
| chr22: g.41093077G > C | A25P | Missense | Substitution |
| chr22: g.41127560C > T | A327V | Missense | Substitution |
| chr22: g.41129892G > A | A391T | Missense | Substitution |
| chr22: g.41131454C > T | A450V | Missense | Substitution |
| chr22: g.41140175C > A | A599D | Missense | Substitution |
| chr22: g.41141049C > T | A627V | Missense | Substitution |
| chr22: g.41117316C > T | A75V | Missense | Substitution |
| chr22: g.41157397T > C | C1164R | Missense | Substitution |
| chr22: g.41157398G > A | C1164Y | Missense | Substitution |
| chr22: g.41160653G > T | C1201F | Missense | Substitution |
| chr22: g.41160653G > A | C1201Y | Missense | Substitution |
| chr22: g.41160662G > A | C1204Y | Missense | Substitution |
| chr22: g.41164064G > T | C1247F | Missense | Substitution |
| chr22: g.41164064G > A | C1247Y | Missense | Substitution |
| chr22: g.41168810G > A | C1372Y | Missense | Substitution |
| chr22: g.41168849G > A | C1385Y | Missense | Substitution |
| chr22: g.41176947T > A | C17465 | Missense | Substitution |
| chr22: g.41176969G > T | C1753F | Missense | Substitution |
| chr22: g.41157262G > A | D1119N | Missense | Substitution |
| chr22: g.41158472G > C | D1188H | Missense | Substitution |
| chr22: g.41160704A > G | D1218G | Missense | Substitution |
| chr22: g.41162760G > T | D1237Y | Missense | Substitution |
| chr22: g.41166609G > A | D1273N | Missense | Substitution |
| chr22: g.41168794G > T | D1367Y | Missense | Substitution |
| chr22: g.41169525G > A | D1399N | Missense | Substitution |
| chr22: g.41169525G > T | D1399Y | Missense | Substitution |
| chr22: g.41170453A > G | D1445G | Missense | Substitution |
| chr22: g.41170452G > A | D1445N | Missense | Substitution |
| chr22: g.41172499G > T | D1485Y | Missense | Substitution |
| chr22: g.41172523G > T | D1493Y | Missense | Substitution |
| chr22: g.41172565G > A | D1507N | Missense | Substitution |
| chr22: g.41176331G > A | D1622N | Missense | Substitution |
| chr22: g.41176773G > C | D1688H | Missense | Substitution |
| chr22: g.41178254C > A | D2181E | Missense | Substitution |
| chr22: g.41178813G > A | D2368N | Missense | Substitution |
| chr22: g.41137751A > G | D574G | Missense | Substitution |
| chr22: g.41140231G > A | D618N | Missense | Substitution |
| chr22: g.41157245A > T | E1113V | Missense | Substitution |
| chr22: g.41160670G > C | E1207Q | Missense | Substitution |
| chr22: g.41160684G > T | E1211D | Missense | Substitution |
| chr22: g.41172559G > A | E1505K | Missense | Substitution |
| chr22: g.41172586G > A | E1514K | Missense | Substitution |
| chr22: g.41172616G > A | E1524K | Missense | Substitution |
| chr22: g.41172624G > C | E1526D | Missense | Substitution |
| chr22: g.41172654A > T | E1536D | Missense | Substitution |
| chr22: g.41172652G > C | E1536Q | Missense | Substitution |
| chr22: g.41178790A > G | E2360G | Missense | Substitution |
| chr22: g.41127658G > A | E360K | Missense | Substitution |
| chr22: g.41093013G > T | E3D | Missense | Substitution |
| chr22: g.41141051G > C | E628Q | Missense | Substitution |
| chr22: g.41141074G > T | E635D | Missense | Substitution |
| chr22: g.41093024A > G | E7G | Missense | Substitution |
| chr22: g.41151857G > A | E948K | Missense | Substitution |
| chr22: g.41158420C > A | F1170L | Missense | Substitution |
| chr22: g.41160651C > A | F1200L | Missense | Substitution |
| chr22: g.41160666C > A | F1205L | Missense | Substitution |
| chr22: g.41164055T > G | F1244C | Missense | Substitution |
| chr22: g.41168723T > G | F1343C | Missense | Substitution |
| chr22: g.41177553T > A | F19481 | Missense | Substitution |
| chr22: g.41177961T > G | F2084V | Missense | Substitution |
| chr22: g.41117189T > G | F33V | Missense | Substitution |
| chr22: g.41157232G > A | G1109R | Missense | Substitution |
| chr22: g.41117433G > A | G114E | Missense | Substitution |
| chr22: g.41168735G > C | G1347A | Missense | Substitution |
| chr22: g.41117517G > T | G142V | Missense | Substitution |
| chr22: g.41170447G > T | G1443V | Missense | Substitution |
| chr22: g.41117546G > T | G152C | Missense | Substitution |
| chr22: g.41177658G > A | G1983R | Missense | Substitution |
| chr22: g.41117694G > A | G201E | Missense | Substitution |
| chr22: g.41178078G > T | G2123W | Missense | Substitution |
| chr22: g.41178310G > C | G2200A | Missense | Substitution |
| chr22: g.41147851G > A | G716S | Missense | Substitution |
| chr22: g.41093030G > T | G9V | Missense | Substitution |
| chr22: g.41160646C > T | H1199Y | Missense | Substitution |
| chr22: g.41164088A > T | H1255L | Missense | Substitution |
| chr22: g.41164088A > G | H1255R | Missense | Substitution |
| chr22: g.41168553C > A | H1327N | Missense | Substitution |
| chr22: g.41170471A > C | H1451P | Missense | Substitution |
| chr22: g.41170471A > G | H1451R | Missense | Substitution |
| chr22: g.41177170A > G | H1820R | Missense | Substitution |
| chr22: g.41178086C > A | H2125Q | Missense | Substitution |
| chr22: g.41178335T > A | H2208Q | Missense | Substitution |
| chr22: g.41127562C > T | H328Y | Missense | Substitution |
| chr22: g.41127620A > T | H347L | Missense | Substitution |
| chr22: g.41127626A > G | H349R | Missense | Substitution |
| chr22: g.41117215C > G | H41Q | Missense | Substitution |
| chr22: g.41137774C > T | H582Y | Missense | Substitution |
| chr22: g.41141060C > G | H631D | Missense | Substitution |
| chr22: g.41149863C > T | H828Y | Missense | Substitution |
| chr22: g.41158463A > G | I1185V | Missense | Substitution |
| chr22: g.41176268A > G | I1601V | Missense | Substitution |
| chr22: g.41176323T > C | I1619T | Missense | Substitution |
| chr22: g.41176915T > C | I1735T | Missense | Substitution |
| chr22: g.41176928C > G | I1739M | Missense | Substitution |
| chr22: g.41177565A > G | I1952V | Missense | Substitution |
| chr22: g.41149800A > G | 1807V | Missense | Substitution |
| chr22: g.41152343G > T | K1045N | Missense | Substitution |
| chr22: g.41162734A > T | K1228I | Missense | Substitution |
| chr22: g.41162735A > C | K1228N | Missense | Substitution |
| chr22: g.41162749A > C | K1233T | Missense | Substitution |
| chr22: g.41166623G > C | K1277N | Missense | Substitution |
| chr22: g.41170495A > T | K1459M | Missense | Substitution |
| chr22: g.41093044A > C | Kl4Q | Missense | Substitution |
| chr22: g.41173655G > T | K1550N | Missense | Substitution |
| chr22: g.41173679A > C | K1558N | Missense | Substitution |
| chr22: g.41173685G > C | K1560N | Missense | Substitution |
| chr22: g.41173708A > G | K1568R | Missense | Substitution |
| chr22: g.41176990A > G | K1760R | Missense | Substitution |
| chr22: g.41177033A > T | K1774N | Missense | Substitution |
| chr22: g.41093053A > G | K17E | Missense | Substitution |
| chr22: g.41140181A > C | K601T | Missense | Substitution |
| chr22: g.41141075A > G | K636E | Missense | Substitution |
| chr22: g.41169522C > A | L13981 | Missense | Substitution |
| chr22: g.41169523T > C | L1398P | Missense | Substitution |
| chr22: g.41169557G > C | L1409F | Missense | Substitution |
| chr22: g.41170507T > G | L1463R | Missense | Substitution |
| chr22: g.41173756T > C | L1584P | Missense | Substitution |
| chr22: g.41176935C > A | L1742M | Missense | Substitution |
| chr22: g.41177065T > C | L1785P | Missense | Substitution |
| chr22: g.41177193C > T | L1828F | Missense | Substitution |
| chr22: g.41177871C > T | L2054F | Missense | Substitution |
| chr22: g.41177872T > G | L2054R | Missense | Substitution |
| chr22: g.41117748T > C | L219P | Missense | Substitution |
| chr22: g.41178591C > A | L2294I | Missense | Substitution |
| chr22: g.41117800G > C | L236F | Missense | Substitution |
| chr22: g.41178828C > A | L2373I | Missense | Substitution |
| chr22: g.41178917G > C | L2402F | Missense | Substitution |
| chr22: g.41178927C > A | L2406I | Missense | Substitution |
| chr22: g.41117199T > G | L36R | Missense | Substitution |
| chr22: g.41137789C > T | L587F | Missense | Substitution |
| chr22: g.41117343T > C | L845 | Missense | Substitution |
| chr22: g.41117369C > T | L93F | Missense | Substitution |
| chr22: g.41168591G > A | M13391 | Missense | Substitution |
| chr22: g.41173722A > G | M1573V | Missense | Substitution |
| chr22: g.41177596T > C | M1962T | Missense | Substitution |
| chr22: g.41177759G > A | M20161 | Missense | Substitution |
| chr22: g.41178219A > G | M2170V | Missense | Substitution |
| chr22: g.41178275G > A | M21881 | Missense | Substitution |
| chr22: g.41178561A > G | M2284V | Missense | Substitution |
| chr22: g.41131545G > A | M4801 | Missense | Substitution |
| chr22: g.41135879T > G | M532R | Missense | Substitution |
| chr22: g.41141138A > C | M657L | Missense | Substitution |
| chr22: g.41149043G > A | M7491 | Missense | Substitution |
| chr22: g.41149041A > G | M749V | Missense | Substitution |
| chr22: g.41157284A > G | N1126S | Missense | Substitution |
| chr22: g.41117449T > A | N119K | Missense | Substitution |
| chr22: g.41168862C > G | N1389K | Missense | Substitution |
| chr22: g.41117590C > A | N166K | Missense | Substitution |
| chr22: g.41177940A > G | N2077D | Missense | Substitution |
| chr22: g.41178091A > T | N21271 | Missense | Substitution |
| chr22: g.41178337A > T | N22091 | Missense | Substitution |
| chr22: g.41178613A > C | N2301T | Missense | Substitution |
| chr22: g.41178820A > C | N2370T | Missense | Substitution |
| chr22: g.41178846A > C | N2379H | Missense | Substitution |
| chr22: g.41125880A > G | N2495 | Missense | Substitution |
| chr22: g.41135833A > T | N517Y | Missense | Substitution |
| chr22: g.41117373A > G | N945 | Missense | Substitution |
| chr22: g.41152324C > T | P1039L | Missense | Substitution |
| chr22: g.41155072C > T | P1074S | Missense | Substitution |
| chr22: g.41164124C > T | P1267L | Missense | Substitution |
| chr22: g.41168755C > T | P1354S | Missense | Substitution |
| chr22: g.41170474C > T | P1452L | Missense | Substitution |
| chr22: g.41172551C > T | P1502L | Missense | Substitution |
| chr22: g.41117561C > G | P157A | Missense | Substitution |
| chr22: g.41117562C > T | P157L | Missense | Substitution |
| chr22: g.41176326C > T | P1620L | Missense | Substitution |
| chr22: g.41177341C > T | P1877L | Missense | Substitution |
| chr22: g.41177473C > T | P1921L | Missense | Substitution |
| chr22: g.41177584C > T | P1958L | Missense | Substitution |
| chr22: g.41177712C > A | P2001T | Missense | Substitution |
| chr22: g.41177893C > T | P2061L | Missense | Substitution |
| chr22: g.41178030C > T | P2107S | Missense | Substitution |
| chr22: g.41178148C > T | P2146L | Missense | Substitution |
| chr22: g.41093065C > G | P21A | Missense | Substitution |
| chr22: g.41178610C > T | P2300L | Missense | Substitution |
| chr22: g.41178609C > T | P2300S | Missense | Substitution |
| chr22: g.41178691C > A | P2327H | Missense | Substitution |
| chr22: g.41178849C > A | P2380T | Missense | Substitution |
| chr22: g.41127532C > G | P318A | Missense | Substitution |
| chr22: g.41127533C > A | P318Q | Missense | Substitution |
| chr22: g.41127575C > T | P332L | Missense | Substitution |
| chr22: g.41127574C > A | P332T | Missense | Substitution |
| chr22: g.41131547C > T | P481L | Missense | Substitution |
| chr22: g.41131604C > T | P500L | Missense | Substitution |
| chr22: g.41137694C > T | P555L | Missense | Substitution |
| chr22: g.41140163C > T | P595L | Missense | Substitution |
| chr22: g.41141181C > T | P671L | Missense | Substitution |
| chr22: g.41149039C > T | P748L | Missense | Substitution |
| chr22: g.41149147C > T | P784L | Missense | Substitution |
| chr22: g.41149899C > A | P840T | Missense | Substitution |
| chr22: g.41149966C > A | P862H | Missense | Substitution |
| chr22: g.41149980C > T | P867S | Missense | Substitution |
| chr22: g.41149993C > A | P871H | Missense | Substitution |
| chr22: g.41150160C > T | P927S | Missense | Substitution |
| chr22: g.41150170C > T | P930L | Missense | Substitution |
| chr22: g.41150179C > T | P933L | Missense | Substitution |
| chr22: g.41160718C > G | Q1223E | Missense | Substitution |
| chr22: g.41168865G > C | Q1390H | Missense | Substitution |
| chr22: g.41170484G > C | Q1455H | Missense | Substitution |
| chr22: g.41170483A > G | Q1455R | Missense | Substitution |
| chr22: g.41177144G > T | Q1811H | Missense | Substitution |
| chr22: g.41177168G > T | Q1819H | Missense | Substitution |
| chr22: g.41177239A > C | Q1843P | Missense | Substitution |
| chr22: g.41177715C > G | Q2002E | Missense | Substitution |
| chr22: g.41177722A > G | Q2004R | Missense | Substitution |
| chr22: g.41178151A > T | Q2147L | Missense | Substitution |
| chr22: g.41178354C > A | Q2215K | Missense | Substitution |
| chr22: g.41178485G > T | Q2258H | Missense | Substitution |
| chr22: g.41117812G > T | Q240H | Missense | Substitution |
| chr22: g.41127516G > T | Q312H | Missense | Substitution |
| chr22: g.41131541A > C | Q479P | Missense | Substitution |
| chr22: g.41149097G > T | Q767H | Missense | Substitution |
| chr22: g.41149765A > G | Q795R | Missense | Substitution |
| chr22: g.41151908C > G | Q965E | Missense | Substitution |
| chr22: g.41155078C > T | R1076C | Missense | Substitution |
| chr22: g.41158470G > A | R1187H | Missense | Substitution |
| chr22: g.41166634G > A | R1281Q | Missense | Substitution |
| chr22: g.41166641G > T | R1283S | Missense | Substitution |
| chr22: g.41168507G > T | R1311S | Missense | Substitution |
| chr22: g.41168509G > A | R1312Q | Missense | Substitution |
| chr22: g.41168762G > A | R1356Q | Missense | Substitution |
| chr22: g.41169560G > T | R1410S | Missense | Substitution |
| chr22: g.41170552G > A | R1478H | Missense | Substitution |
| chr22: g.41172527G > C | R1494T | Missense | Substitution |
| chr22: g.41176346C > G | R1627G | Missense | Substitution |
| chr22: g.41176346C > T | R1627W | Missense | Substitution |
| chr22: g.41176401G > A | R1645Q | Missense | Substitution |
| chr22: g.41176505C > T | R1680C | Missense | Substitution |
| chr22: g.41176920C > T | R1737C | Missense | Substitution |
| chr22: g.41176957G > A | R1749Q | Missense | Substitution |
| chr22: g.41177196C > T | R1829C | Missense | Substitution |
| chr22: g.41177201G > T | R1830S | Missense | Substitution |
| chr22: g.41177973C > T | R2088W | Missense | Substitution |
| chr22: g.41178633C > T | R2308C | Missense | Substitution |
| chr22: g.41178661G > T | R2317L | Missense | Substitution |
| chr22: g.41178661G > A | R2317Q | Missense | Substitution |
| chr22: g.41127584G > A | R335H | Missense | Substitution |
| chr22: g.41127689G > A | R370H | Missense | Substitution |
| chr22: g.41131616G > T | R504L | Missense | Substitution |
| chr22: g.41137732C > T | R568W | Missense | Substitution |
| chr22: g.41137769G > A | R580Q | Missense | Substitution |
| chr22: g.41140190G > A | R604Q | Missense | Substitution |
| chr22: g.41140216C > T | R613W | Missense | Substitution |
| chr22: g.41146769G > A | R695H | Missense | Substitution |
| chr22: g.41146798C > G | R705G | Missense | Substitution |
| chr22: g.41146799G > A | R705Q | Missense | Substitution |
| chr22: g.41149893C > T | R838C | Missense | Substitution |
| chr22: g.41117349G > A | R86Q | Missense | Substitution |
| chr22: g.41150125G > T | R915L | Missense | Substitution |
| chr22: g.41152234C > A | S1009Y | Missense | Substitution |
| chr22: g.41117408A > T | S106C | Missense | Substitution |
| chr22: g.41157191G > T | S10951 | Missense | Substitution |
| chr22: g.41160692C > T | S1214F | Missense | Substitution |
| chr22: g.41160692C > A | S1214Y | Missense | Substitution |
| chr22: g.41117489T > G | S133A | Missense | Substitution |
| chr22: g.41169529G > T | S14001 | Missense | Substitution |
| chr22: g.41169530T > G | 51400R | Missense | Substitution |
| chr22: g.41173695A > G | 51564G | Missense | Substitution |
| chr22: g.41173735C > T | 51577F | Missense | Substitution |
| chr22: g.41173747C > T | S1581L | Missense | Substitution |
| chr22: g.41177734C > T | 52008F | Missense | Substitution |
| chr22: g.41177995C > A | 52095Y | Missense | Substitution |
| chr22: g.41178808G > A | 52366N | Missense | Substitution |
| chr22: g.41178832C > A | 52374Y | Missense | Substitution |
| chr22: g.41178844G > A | 52378N | Missense | Substitution |
| chr22: g.41178909T > A | 52400T | Missense | Substitution |
| chr22: g.41129908C > G | 5396C | Missense | Substitution |
| chr22: g.41131493C > T | 5463F | Missense | Substitution |
| chr22: g.41146760G > T | S692I | Missense | Substitution |
| chr22: g.41149774G > A | 5798N | Missense | Substitution |
| chr22: g.41149879C > T | S833L | Missense | Substitution |
| chr22: g.41151887A > G | S95 8G | Missense | Substitution |
| chr22: g.41151891G > A | 5959N | Missense | Substitution |
| chr22: g.41151906C > A | 5964Y | Missense | Substitution |
| chr22: g.41151933C > T | 5973F | Missense | Substitution |
| chr22: g.41152243C > T | T1012I | Missense | Substitution |
| chr22: g.41157209C > T | T1101I | Missense | Substitution |
| chr22: g.41160721A > G | T1224A | Missense | Substitution |
| chr22: g.41169561A > G | T1411A | Missense | Substitution |
| chr22: g.41176791A > G | T1694A | Missense | Substitution |
| chr22: g.41177302C > G | T1864S | Missense | Substitution |
| chr22: g.41178883C > T | T2391M | Missense | Substitution |
| chr22: g.41125907C > T | T258I | Missense | Substitution |
| chr22: g.41117400T > G | V103G | Missense | Substitution |
| chr22: g.41157373G > A | V1156M | Missense | Substitution |
| chr22: g.41160688G > A | V12131 | Missense | Substitution |
| chr22: g.41168551T > C | V1326A | Missense | Substitution |
| chr22: g.41168725G > A | V1344M | Missense | Substitution |
| chr22: g.41177002T > G | V1764G | Missense | Substitution |
| chr22: g.41178081G > A | V2124I | Missense | Substitution |
| chr22: g.41149968G > C | V863L | Missense | Substitution |
| chr22: g.41157272G > T | W1122L | Missense | Substitution |
| chr22: g.41157294G > T | W1129C | Missense | Substitution |
| chr22: g.41170425T > C | W1436R | Missense | Substitution |
| chr22: g.41170425T > A | W1436R | Missense | Substitution |
| chr22: g.41170517G > C | W1466C | Missense | Substitution |
| chr22: g.41170516G > T | W1466L | Missense | Substitution |
| chr22: g.41172573G > T | W1509C | Missense | Substitution |
| chr22: g.41172571T > C | W1509R | Missense | Substitution |
| chr22: g.41172571T > A | W1509R | Missense | Substitution |
| chr22: g.41157239A > G | Y1111C | Missense | Substitution |
| chr22: g.41157323A > G | Y1139C | Missense | Substitution |
| chr22: g.41160644A > T | Y1198F | Missense | Substitution |
| chr22: g.41169520A > G | Y1397C | Missense | Substitution |
| chr22: g.41169519T > G | Y1397D | Missense | Substitution |
| chr22: g.41169571A > G | Y1414C | Missense | Substitution |
| chr22: g.41169570T > G | Y1414D | Missense | Substitution |
| chr22: g.41170456A > G | Y1446C | Missense | Substitution |
| chr22: g.41170519A > T | Y1467F | Missense | Substitution |
| chr22: g.41170519A > C | Y14675 | Missense | Substitution |
| chr22: g.41117712A > G | Y207C | Missense | Substitution |
| chr22: g.41125903T > G | Y257D | Missense | Substitution |
| chr22: g.41140210T > C | Y611H | Missense | Substitution |
| chr22: g.41154935delTTTTTTAAAG | X1048_splice | Splice acc. var. | Deletion |
| TTCTTCTGCTTAATTGGTAACTAATTTCAA | | | |
| ATGCACTTTTTTTTTTTAAGT | | | |
| chr22: g.41157158delTGTCTTTCTA | X1088_splice | Splice acc. var. | Deletion |
| GGATTACTTTGATA | | | |
| chr22: g.41157155delACTTGTCTTT | X1088_splice | Splice acc. var. | Deletion |
| CTAGGATT | | | |
| chr22: g.41157138delGAGTAATGTT | X1088_splice | Splice acc. var. | Deletion |
| TGATGTCACTTGTCTTTCTAG | | | |
| chr22: g.41160641G > T | X1197_splice | Splice acc. var. | Substitution |
| chr22: g.41164051A > T | X1243_splice | Splice acc. var. | Substitution |
| chr22: g.41164052G > A | X1243_splice | Splice acc. var. | Substitution |
| chr22: g.41166598G > C | X1269_splice | Splice acc. var. | Substitution |
| chr22: g.41168719A > G | X1342_splice | Splice acc. var. | Substitution |
| chr22: g.41169497delGTATAGGAGA | X1391_splice | Splice acc. var. | Deletion |
| chr22: g.41170405G > C | X1429_splice | Splice acc. var. | Substitution |
| chr22: g.41172497A > G | X1485_splice | Splice acc. var. | Substitution |
| chr22: g.41172495delACAGGATATT | X1485_splice | Splice acc. var. | Deletion |
| TTTAAACAAGCTACTGAAGATAGATTA | | | |
| chr22: g.41176246G > A | X1594_splice | Splice acc. var. | Substitution |
| chr22: g.41117185A > G | X32_splice | Splice acc. var. | Substitution |
| chr22: g.41135812G > T | X510_splice | Splice acc. var. | Substitution |
| chr22: g.41135811A > G | X510_splice | Splice acc. var. | Substitution |
| chr22: g.41137651A > G | X541_splice | Splice acc. var. | Substitution |
| chr22: g.41147836G > A | X711_splice | Splice acc. var. | Substitution |
| chr22: g.41147836G > T | X711_splice | Splice acc. var. | Substitution |
| chr22: g.41160723G > T | X1224_splice | Splice don. var. | Substitution |
| chr22: g.41160723G > A | X1224_splice | Splice don. var. | Substitution |
| chr22: g.41162775delGAACTGTAAG | X1242_splice | Splice don. var. | Deletion |
| TACGATCCCCTTGAATAGTCAGTACGCTTT | | | |
| GGCTTTTCTTTTTCCCTTTCATTCTCTTGA | | | |
| A | | | |
| chr22: g.41172664G > A | X1539_splice | Splice don. var. | Substitution |
| chr22: g.41131635T > C | X510_splice | Splice don. var. | Substitution |
| chr22: g.41140140C > T | L587L | Splice reg. var. | Substitution |
| chr22: g.41151835T > G | L940L | Splice reg. var. | Substitution |
| chr22: g.41147946G > A | P747P | Splice reg. var. | Substitution |
| chr22: g.41176329_41176330insG | C1621delinsW*TPIYIGVQ | Truncation | Insertion |
| TGAACACCTATATACATAGGTGTTCAAAGT | SI | | |
| AT | | | |
| chr22: g.41177024C > A | C1771* | Truncation | Substitution |
| chr22: g.41152260G > T | E1018* | Truncation | Substitution |
| chr22: g.41157346G > T | E1147* | Truncation | Substitution |
| chr22: g.41162736G > T | E1229* | Truncation | Substitution |
| chr22: g.41166645G > T | E1285* | Truncation | Substitution |
| chr22: g.41168746G > T | E1351* | Truncation | Substitution |
| chr22: g.41172559G > T | E1505* | Truncation | Substitution |
| chr22: g.41172613G > T | E1523* | Truncation | Substitution |
| chr22: g.41176385G > T | E1640* | Truncation | Substitution |
| chr22: g.41176481G > T | E1672* | Truncation | Substitution |
| chr22: g.41178465G > T | E2252* | Truncation | Substitution |
| chr22: g.41093011G > T | E3* | Truncation | Substitution |
| chr22: g.41140225G > T | E616* | Truncation | Substitution |
| chr22: g.41151920G > T | E969* | Truncation | Substitution |
| chr22: g.41178357G > T | G2216* | Truncation | Substitution |
| chr22: g.41135827G > T | G515* | Truncation | Substitution |
| chr22: g.41135845G > T | G521* | Truncation | Substitution |
| chr22: g.41151860G > T | G949* | Truncation | Substitution |
| chr22: g.41152347_41152348insC | K1047delinsT*EFLVFQ | Truncation | Insertion |
| GTAAGAATTTTTAGTATTCC | | | |
| chr22: g.41152314C > T | Q1036* | Truncation | Substitution |
| chr22: g.41155096C > T | Q1082* | Truncation | Substitution |
| chr22: g.41157241C > T | Q1112* | Truncation | Substitution |
| chr22: g.41160718C > T | Q1223* | Truncation | Substitution |
| chr22: g.41170509C > T | Q1464* | Truncation | Substitution |
| chr22: g.41176866C > T | Q1719* | Truncation | Substitution |
| chr22: g.41177007C > T | Q1766* | Truncation | Substitution |
| chr22: g.41117642C > T | Q184* | Truncation | Substitution |
| chr22: g.41177853C > T | Q2048* | Truncation | Substitution |
| chr22: g.41178072C > T | Q2121* | Truncation | Substitution |
| chr22: g.41178210C > T | Q2167* | Truncation | Substitution |
| chr22: g.41178354C > T | Q2215* | Truncation | Substitution |
| chr22: g.41117762C > T | Q224* | Truncation | Substitution |
| chr22: g.41178483C > T | Q2258* | Truncation | Substitution |
| chr22: g.41178573C > T | Q2288* | Truncation | Substitution |
| chr22: g.41178603C > T | Q2298* | Truncation | Substitution |
| chr22: g.41125909C > T | Q259* | Truncation | Substitution |
| chr22: g.41125957C > T | Q275* | Truncation | Substitution |
| chr22: g.41127667C > T | Q363* | Truncation | Substitution |
| chr22: g.41135851C > T | Q523* | Truncation | Substitution |
| chr22: g.41137711C > T | Q561* | Truncation | Substitution |
| chr22: g.41117285C > T | Q65* | Truncation | Substitution |
| chr22: g.41147905C > T | Q734* | Truncation | Substitution |
| chr22: g.41147926C > T | Q741* | Truncation | Substitution |
| chr22: g.41149065C > T | Q757* | Truncation | Substitution |
| chr22: g.41149095C > T | Q767* | Truncation | Substitution |
| chr22: g.41149818C > T | Q813* | Truncation | Substitution |
| chr22: g.41149866C > T | Q829* | Truncation | Substitution |
| chr22: g.41151908C > T | Q965* | Truncation | Substitution |
| chr22: g.41166633C > T | R1281* | Truncation | Substitution |
| chr22: g.41168508C > T | R1312* | Truncation | Substitution |
| chr22: g.41168761C > T | R1356* | Truncation | Substitution |
| chr22: g.41170503C > T | R1462* | Truncation | Substitution |
| chr22: g.41172631C > T | R1529* | Truncation | Substitution |
| chr22: g.41176400C > T | R1645* | Truncation | Substitution |
| chr22: g.41178264C > T | R2185* | Truncation | Substitution |
| chr22: g.41178498C > T | R2263* | Truncation | Substitution |
| chr22: g.41129910C > T | R397* | Truncation | Substitution |
| chr22: g.41141111C > T | R648* | Truncation | Substitution |
| chr22: g.41152303_41152304insT | S1033Cfs*12 | Truncation | Insertion |
| TGTATGAGCCACCACGCCTGGCAATGGTTG | | | |
| TTTTAGATTA | | | |
| chr22: g.41093060C > G | S19* | Truncation | Substitution |
| chr22: g.41117676C > G | S195* | Truncation | Substitution |
| chr22: g.41093075C > A | S24* | Truncation | Substitution |
| chr22: g.41178910C > G | S2400* | Truncation | Substitution |
| chr22: g.41125976C > A | S281* | Truncation | Substitution |
| chr22: g.41125976C > G | S281* | Truncation | Substitution |
| chr22: g.41129923C > G | S401* | Truncation | Substitution |
| chr22: g.41151870C > G | S952* | Truncation | Substitution |
| chr22: g.41129930G > A | W403* | Truncation | Substitution |
| chr22: g.41137742G > A | W571* | Truncation | Substitution |
| chr22: g.41157240T > G | Y1111* | Truncation | Substitution |
| chr22: g.41141056C > G | Y629* | Truncation | Substitution |

*Mutations are annotated according the ENSEMBL ENSG00000100393, Chromosome 22:
41,091,786-41,180,079 forward strand, build GRCh38: CM000684.2, last accessed Jul. 24, 2017.

In some embodiments, the EP300 loss of function mutation is heterozygous, e.g., only one allele of EP300 is affected by a loss of function mutation, while the other allele is not affected by a loss of function mutation. In some embodiments, however, both EP300 alleles are affected by a loss of function mutation. In some such embodiments, at least one loss of function mutation is homozygous, i.e., it affects both alleles. In some embodiments, each EP300 allele is affected by a different loss of function mutation, or a different combination of EP300 loss of function mutations.

The nucleic acid and protein sequences presented herein, as well as the mutations described herein, are exemplary and are not meant to limit the scope of this disclosure. Additional suitable sequences and additional suitable EP300 loss of function mutations will be apparent to those of ordinary skill in the art based on the instant disclosure and the general knowledge in the art, or can be identified by the skilled artisan based on the teachings of the present specification with no more than routine experimentation. The disclosure is not limited in this respect.

### Cancers and Tumors

The present disclosure provides, inter alia, methods and compositions useful in the treatment of cancer, e.g., for the treatment of a tumor in a subject. In some embodiments, the cancer or tumor comprises an EP300 loss of function, e.g., an EP300 loss of function mutation, or a decreased expression and/or activity level of EP300 protein, e.g., as compared to a reference level, such as, e.g., an EP300 expression and/or activity level observed or expected in a non-cancerous or non-tumor cell of the same tissue of origin as the cancer or tumor.

Cancers that can exhibit an EP300 loss of function, e.g., mediated by an EP300 loss of function mutation described herein, and that are thus sensitive to treatment with CREBBP inhibition therapy, and that can thus be treated with the methods and compositions provided herein, include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some embodiments, the present disclosure provides methods and compositions to treat a cancer in a subject exhibiting an EP300 loss of function, wherein the cancer is endometrial carcinoma, bladder urothelial carcinoma, cervical squamous cell carcinoma, endocervical adenocarcinoma, colon adenocarcinoma, head and neck squamous cell carcinoma, stomach adenocarcinoma, skin cutaneous melanoma, esophageal carcinoma, lymphoid neoplasm, diffuse large b-cell lymphoma, rectum adenocarcinoma, lung squamous cell carcinoma, kidney renal papillary cell carcinoma, cholangiocarcinoma, glioblastoma multiforme, liver hepatocellular carcinoma, ovarian serous cystadenocarcinoma, sarcoma, thymoma, breast invasive carcinoma, lung adenocarcinoma, pancreatic adenocarcinoma, kidney renal clear cell carcinoma, uterine carcinosarcoma, acute myeloid leukemia, uveal melanoma, mesothelioma, prostate adenocarcinoma, adrenocortical carcinoma, testicular germ cell tumors, or brain lower grade glioma.

In some embodiments, the present disclosure provides methods and compositions for treating a tumor in a subject. In some embodiments, the tumor is a solid tumor. In some embodiments, the tumor is a liquid or disperse tumor. In some embodiments, the tumor or a cell comprised in the tumor harbors an EP300 loss of function mutation. In some embodiments, the tumor is associated with a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, follicular lymphoma, diffuse large B-cell lymphoma, Langerhans cell histiocytosis, multiple myeloma, or myeloproliferative neoplasms.

In some embodiments, a tumor comprises a solid tumor. In some embodiments, solid tumors include but are not limited to tumors of the bladder, breast, central nervous system, cervix, colon, esophagus, endometrium, head and neck, kidney, liver, lung, ovary, pancreas, skin, stomach, uterus, or upper respiratory tract. In some embodiments, a tumor that may be treated by the compositions and methods of the present disclosure is a breast tumor. In some embodiments, a tumor that may be treated by the compositions and methods of the present disclosure is not a lung tumor.

In some embodiments, a tumor or cancer suitable for treatment with the methods and compositions provided herein includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma, Atypical Rhabdoid Tumor, Basal Cell Carcinoma, Bile Duct Cancer, Bladder Cancer, Bone Cancer, Brain Tumor, Breast Cancer, Bronchial Tumor, Burkitt Lymphoma, Carcinoid Tumor, Carcinoma, Cardiac (Heart) Tumor, Central Nervous System Tumor, Cervical Cancer, Cholangiocarcinoma, Chordoma, Chronic Lymphocytic Leukemia (CLL), Chronic Myelogenous Leukemia (CML), Chronic Myeloproliferative Neoplasm, Colorectal Cancer, Craniopharyngioma, Cutaneous T-Cell Lymphoma, Ductal Carcinoma In Situ (DCIS), Embryonal Tumor, Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Tumor, Kaposi Sarcoma, Kidney Tumor, Langerhans Cell Histiocytosis, Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome, Myelodysplastic/Myeloproliferative Neoplasm, Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer, Renal Cell (Kidney) Cancer, Retinoblastoma, Retinoblastoma, Rhabdomyosarcoma, Rhabdomyosarcoma, Salivary Gland Cancer, Sarcoma, Sézary Syndrome, Skin Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenström Macroglobulinemia, Wilms' Tumor.

Some aspects of this disclosure provide that a cancer or tumor exhibiting a loss of function of EP300 is sensitive to CREBBP inhibition treatment. In some embodiments, the cancer or tumor exhibits an EP300 loss of function mutation. In some embodiments, the cancer or tumor exhibits a loss of function mutation as described herein. In some embodiments, the cancer or tumor exhibits an EP300 mutation that results in a truncation of the EP300 HAT domain, or in a missense mutation within the EP300 HAT domain. In some embodiments, the cancer or tumor exhibits loss of wild-type EP300 expression. In some embodiments, the cancer or tumor comprises a mutant allele of EP300, e.g., an allele harboring a loss-of-function mutation of EP300, and exhibits loss of wild-type expression of EP300 protein. In some such embodiments, the cancer or tumor harbors a wild-type EP300 allele, but does not express wild-type EP300 from the wild-type allele. In some embodiments, the wild-type EP300 allele is silenced, e.g., via epigenetic mechanisms. In some embodiments, EP300 expression from the wild-type allele is decreased or abolished through transcriptional repression, or through post-transcriptional or post-translational mechanisms. In some embodiments, each EP300 allele of the cancer or tumor is affected by at least one EP300 loss of function mutation.

Some aspects of this disclosure provide that, in some embodiments, a cancer or tumor harboring a loss of function mutation in an EP300 gene is sensitive to treatment with CREBBP inhibition therapy. Accordingly, in some embodiments, the cancer or tumor treated with the compositions or according to the methods provided herein is an EP300 mutant cancer or tumor. In other embodiments, the cancer or tumor does not harbor an EP300 loss of function mutation. In some such embodiments, the cancer or tumor harbors an EP300 loss of function that is mediated by epigenetic mechanisms, e.g., by silencing of EP300, or by post-transcriptional and/or post-translational silencing.

In some particular embodiments, the present disclosure provides therapies for tumors with mutations in EP300. In some embodiments, methods and compositions of the present disclosure are not used in treatment of tumors harboring one or more particular CREBBP mutations. In some embodiments, methods and compositions of the present disclosure are not used in treatment of hematopoietic tumors deficient in CREBBP.

### Detecting Sensitivity

In some embodiments, the present disclosure defines subjects, cancers, and/or tumors susceptible or sensitive to treatment with CREBBP inhibition therapy. In some embodiments, the present disclosure provides technologies for identifying and/or characterizing such sensitive subjects, cancers, and/or tumors. In some embodiments, the present disclosure provides technologies for detecting sensitivity to treatment with CREBBP inhibition therapy.

In some embodiments, the provided technologies comprise detecting in a sample from a subject a mutant gene or gene product, e.g., a mutant EP300 gene or gene product comprising a loss-of-function mutation. In some embodiments, a sample comprises a blood, serum, tissue, or tumor sample. For example, in some embodiments, the provided technologies involve obtaining and/or analyzing a tumor biopsy sample. In some embodiments, a sample comprises, tumor cells or tumor cell components (e.g., disrupted tumor cells or a cellular lysate from disrupted tumor cells). In some embodiments, a sample contains nucleic acid from a tumor, e.g., tumor DNA or RNA. In some embodiments, a sample contains polypeptide from a tumor.

In some embodiments, the present disclosure establishes that tumors characterized by reduced level and/or activity of EP300, or harboring a loss-of-function mutation in an EP300 gene or gene product, are sensitive to CREBBP inhibition therapy.

In some embodiments, such sensitivity to CREBBP inhibition therapy is associated with presence of one or more loss-of-function mutations and/or deletions in EP300, e.g., with one or more of the loss-of function mutations provided herein. For example, in some embodiments, an EP300 mutation associated with sensitivity to CREBBP inhibition therapy can include, for example, V5L, C1201Y, C1385Y, T329R, D1399N, D1399Y, S1650Y A1437V, splice variation at G711, K1468fs, K1488fs, K291fs, R1234fs, Y1467fs, P1081S, P802L, R1055*, R1645*, Q1874E, Q2023*, Q2306E, Q993*, R397*, R86*, R1950G, S1754*, W1509C, G1042*, Y1414C, or combinations thereof.

For example, in some embodiments, an EP300 mutation associated with sensitivity to CREBBP inhibition therapy can include, for example, G30V, K423T, R883G, T891P, P2097A, E1014*, or Q1661*.

In some embodiments, an EP300 mutation associated with sensitivity to CREBBP inhibition therapy can include any EP300 mutation provided herein, e.g., as listed in Table 4 or described in any of the drawings, or a combination of such mutations. Additional suitable EP300 mutations will be apparent to the skilled artisan based on the instant disclosure and the general knowledge in the art. The present disclosure is not limited in this respect.

In some embodiments, an EP300 mutation associated with sensitivity to CREBBP inhibition therapy as described herein may be characterized by a variation in DNA copy number relative to a reference. In some embodiments, such an EP300 mutation is characterized by a variation in mRNA expression level relative to a reference. In some embodiments, a reference is a historical reference, a population based reference, or a subject specific reference. In some embodiments, a reference is determined from a sample of nucleic acid from a subject's tissue other than the tumor.

In some embodiments, an EP300 mutation associated with sensitivity to CREBBP inhibition therapy as described herein comprises a frame shift mutation, a splice variant, a missense mutation, a nonsense mutation, an insertion, a deletion, or combinations thereof. In some embodiments, a frame shift mutation (fs) is a mutation caused by an insertion or deletion of nucleotides resulting in a shift of the reading frame of the DNA. In some embodiments, a splice variant arises from a mutation that results in splicing not observed or not observed frequently in the absence of mutation. In some embodiments, a missense mutation is a single nucleotide change that results in a codon which codes for a different amino acid than the amino acid coded for in the absence of the mutation. In some embodiments, a nonsense mutation is a mutation that results in a stop codon (exemplified herein by “*”).

In some embodiments, a subject, or a cancer cell within a subject, sensitive to CREBBP inhibition therapy harbors an EP300 mutant comprising a mutation in the KIX domain, the bromodomain, or the HAT domain of EP300. In some embodiments, a subject having or diagnosed with a cancer is determined to be sensitive to treatment with CREBBP inhibition therapy as provided herein, based on the subject, or a cancer cell within the subject, harboring a mutation in one or more of the following residues in the EP300 protein sequence provided in SEQ ID NO: 3, or of a residue equivalent thereof: V5, R86, K291, T329, R397, G711, P802, P1081, Q993, G1042, R1055, C1201, R1234, C1385, D1399, Y1414, A1437, Y1467, K1468, K1488, W1509, R1645, S1650, S1754, Q1874, R1950, Q2023, and Q2306.

In some embodiments, a subject having or diagnosed with a cancer is determined to be sensitive to treatment with CREBBP inhibition therapy as provided herein, based on the subject, or a cancer cell within the subject, harboring a mutation in one or more of the following residues in the EP300 protein sequence provided in SEQ ID NO: 3, or of a residue equivalent thereof: G30, K423, R883, T891, E1014, Q1661, and P2097.

For example, in some embodiments, the subject or a cancer cell within the subject, harbors one or more of the following mutations in the EP300 protein sequence provided in SEQ ID NO: 3, or a functionally equivalent mutation: a V5L, T329R, P802L, P1081S, C1201Y, C1385Y, D1399N, D1399Y, Y1414C, A1437V, W1509C, 51650Y, Q1874E, R1950G, or Q2306E substitution; a K291fs, R1234fs, K1468fs, K1488fs, or Y1467fs frameshift mutation; a R86*, R397*, Q993*, G1042*, R1055*, R1645*, S1754*, or Q2023* truncation; or a splice variation at G711.

For example, in some embodiments, the subject or a cancer cell within the subject, harbors one or more of the following mutations in the EP300 protein sequence provided in SEQ ID NO: 3, or a functionally equivalent mutation: G30V, K423T, R883G, T891P, P2097A, E1014*, or Q1661*.

In some embodiments, a subject having or diagnosed with a cancer is determined to be sensitive to treatment with CREBBP inhibition therapy as provided herein, based on the subject, or a cancer cell within the subject, harboring an EP300 mutation provided herein, e.g., anywhere in the specification, in Table 4, or in any of the drawings, or any combination of such mutations. In some embodiments, the present disclosure provides methods that comprise determining whether a subject or a cancer is sensitive to treatment with CREBBP inhibition therapy as provided herein based on the presence of a loss-of-function mutation in an EP300 gene or gene product, or on the presence of reduces EP300 activity, or an abolishment of EP300 activity, within the subject or within cancer cells in the subject. In some embodiments, the method further comprises detecting the loss-of-function mutation and/or the reduced or abolished EP300 activity in the subject or in cancer cells of the subject, for example, by analyzing a biological sample obtained from the subject. In some embodiments, the method further comprises obtaining the sample from the subject. In some embodiments, the method further comprises administering CREBBP inhibition therapy to a subject, if the subject, or a cancer within the subject, has been determined to be sensitive to treatment with CREBBP inhibition therapy.

In some embodiments, the present disclosure provides technologies for detecting reduced EP300 level or activity in a sample. In some embodiments, the present disclosure provides technologies for detecting presence of one or more EP300 mutations (e.g., particular loss-of-function mutations or deletions) in a sample.

In some embodiments, an EP300 mutation described herein comprises an alteration at a site that is upstream, downstream, or within the EP300 coding region; in some embodiments, an EP300 mutation described herein comprises an alteration at a site that is upstream, downstream, or within the HAT domain of EP300. In some embodiments, an EP300 mutation as described herein is characterized by disruption of a HAT domain. In some embodiments, an EP300 mutation as described herein is characterized by disruption or loss of a HAT domain (e.g. the HAT domain is totally and or partially absent from EP300). In some embodiments, an EP300 mutation as described herein is characterized by a mutation within a HAT domain. In some embodiments, an EP300 mutation as described herein is characterized by a mutation upstream of a HAT domain. In some embodiments, an EP300 mutation as described herein is characterized by a mutation downstream of a HAT domain. In some embodiments, an EP300 mutation described herein comprises an alteration at a site that is within an EP300 regulatory region (e.g., promoter, enhancer, splice site, termination site). In some embodiments, a mutant form of a gene or gene product is detected in a nucleic acid (e.g., chromosomal DNA, genomic DNA, pre-mRNA, mRNA, cDNA) by, for example, Sanger dideoxy sequencing, pyrosequencing, next generation sequencing-amplicon capture, next generation sequencing-hybridization capture, next generation sequencing-whole exome sequencing, next generation sequencing-whole genome sequencing, digital droplet PCR, Beads, Emulsification, Amplification, and Magnetics (e.g. “BEAMing”), single base extension assay, restriction fragment length polymorphism (RFLP), multiplex ligation-dependent probe amplification (MLPA), single-strand conformation polymorphism (SSCP), denaturing gradient gel electrophoresis (DGGE), microarray, allelic specific PCR, fluorescence in situ hybridization (FISH), mass spectroscopy. In some embodiments, a mutant form of a gene or gene product is detected in a polypeptide by, for example, mass spectroscopy, HPLC, Western blotting including far Western, immunoprecipitations, enzymatic activity assays or combinations thereof. Additional suitable methods for detecting mutants will be apparent to the skilled artisan based on the instant disclosure and the general knowledge in the art. The present disclosure is not limited in this respect.

Some aspects of this disclosure provide that certain cancers or tumors are sensitive to CREBBP inhibition therapy based on the cancer or tumor being characterized by a loss of function of EP300. In some embodiments, the loss of function of EP300 is caused by a loss of function mutation, e.g., a loss of function mutation described herein. In some embodiments, however, the cancer or tumor is characterized by EP300 loss of function not associated with a known loss of function mutation. For example, in some such embodiments, EP300 protein level in the tumor or cancer, or in a subtype or subpopulation of tumor or cancer cells, is reduced as compared to a reference level, e.g., as compared to normal, non-malignant cells of the same tissue origin. In some such embodiments, loss of EP300 function may be the result of epigenetic silencing of the EP300 gene, or of a component of the transcription or translation machinery involved in the expression of EP300. In some embodiments, loss of EP300 function may be the result of an elevated level of EP300 degradation. Regardless of the underlying cause for loss of EP300 function, the reduced or abolished transcript or protein expression level or the reduced or abolished function of EP300 can be detected by appropriate assays that will be apparent to those of skill in the art. Such assays include, for example, microarray, Q-PCR, mass spectroscopy, HPLC, Western blotting, immunoprecipitation, enzymatic activity assays, fluorogenic assays, ELISA assays, AlphaLisa assays, or combinations thereof. Additional suitable methods for detecting EP300 loss of function on the genomic, transcriptional, or protein expression or functional levels will be apparent to the skilled artisan based on the instant disclosure and the general knowledge in the art. The present disclosure is not limited in this respect.

Some aspects of this disclosure provide methods useful for identifying a subject having cancer or a tumor as sensitive to treatment with CREBBP inhibition therapy based on the subject, the cancer, or the tumor exhibiting an EP300 loss of function. Some aspects of this disclosure provide diagnostic methods comprising detecting an EP300 loss of function in a cancer or tumor, wherein the cancer or tumor is identified as a cancer or tumor sensitive to CREBBP inhibition therapy, if a loss of function of EP300 is detected, e.g., by detecting an EP300 loss of function mutation, a reduction of EP300 expression in the cancer or tumor, and/or a reduction of EP300 activity in the cancer or tumor. Some aspects of this disclosure provide methods that comprise detecting an EP300 loss of function in a cancer or tumor, wherein the cancer or tumor is identified to be sensitive to CREBBP inhibition treatment if a loss of function of EP300 is detected in the cancer or tumor, or in a cancer cell or tumor cell, or a cancer cell population or tumor cell population. In some embodiments, the method comprises obtaining a sample comprising a cancer cell or a tumor cell, or a cancer cell population or tumor cell population, and detecting a level of an EP300 gene product (e.g., an EP300 transcript or EP300 protein level), an EP300 loss of function mutation, or a level of EP300 enzymatic activity in the sample. In some embodiments, the EP300 expression or activity level is compared to a reference level, e.g., to a level observed or expected in a sample of similar properties but known to not contain cancer or tumor cells or cell populations, e.g., a sample of cells or cell populations from the same tissue as the tissue of origin of the cancer or tumor. In some embodiments, if a loss of function of EP300 is detected in the cancer or tumor, or in a cancer cell or tumor cell, as compared to the reference, the cancer or tumor is identified as sensitive to CREBBP inhibition treatment. In some embodiments, detecting the EP300 loss of function comprises detecting an EP300 loss of function mutation, e.g., a mutation described herein or otherwise known to those of skill in the art. In some embodiments, detecting an EP300 loss of function mutation comprises detecting whether the mutation is heterozygous or homozygous. In some embodiments, the mutation is heterozygous. In some embodiments, detecting an EP300 loss of function comprises detecting an expression level of an EP300 gene product, e.g., an EP300 transcript or an EP300 protein. In some embodiments, an EP300 loss of function is a decrease of EP300 expression level of at least 10%, at least 20%, at least 25%, at least 30%, at least 40%, at least 50%, at least 60%, at least 70%, at least 75%, at least 80%, at least 90%, at least 95%, at least 97%, at least 98%, or at least 99% e.g., as compared to the expression level of EP300 in a normal cell of the same tissue of origin as the cancer or tumor. In some embodiments, an EP300 loss of function is a complete abolishment of EP300 expression, e.g., a decrease below detectable levels. In some embodiments, detecting an EP300 loss of function comprises determining a level of EP300 activity in a cancer or tumor, e.g., in a cancer cell or a tumor cell, or in a cancer cell population or tumor cell population obtained from a subject having the cancer or tumor. EP300 mutations, expression levels, and activity levels can be measured by any suitable method provided herein or otherwise known to those of ordinary skill in the art. Suitable methods include, without limitation, Sanger dideoxy sequencing, pyrosequencing, next generation sequencing-amplicon capture, next generation sequencing-hybridization capture, next generation sequencing-whole exome sequencing, next generation sequencing-whole genome sequencing, digital droplet PCR, Beads, Emulsification, Amplification, and Magnetics (e.g. “BEAMing”), single base extension assay, restriction fragment length polymorphism (RFLP), multiplex ligation-dependent probe amplification (MLPA), single-strand conformation polymorphism (SSCP), denaturing gradient gel electrophoresis (DGGE), microarray, allelic specific PCR, fluorescence in situ hybridization (FISH), mass spectroscopy, HPLC, Western blotting including far Western, immunoprecipitation, enzymatic activity assays, e.g., fluorogenic activity assays, isotope incorporation assays, fluorescence polarization assays, ELISA assays, AplphaLisa assays, or combinations of such assays. Additional suitable methods for detecting EP300 loss of function will be apparent to the skilled artisan based on the instant disclosure and the general knowledge in the art. The present disclosure is not limited in this respect.

In some embodiments, methods for determining sensitivity of a cancer or tumor to CREBBP inhibition therapy are provided. In some aspects, such methods are based on the recognition that cancer cells or tumor cells harboring an EP300 loss of function mutation in at least one EP300 allele, and exhibiting a loss of wild-type EP300 expression (e.g., from an allele not affected by the EP300 loss of function mutation), are sensitive to treatment with CREBBP treatment. In some embodiments, the method comprises (a) detecting a loss of function mutation of EP300 in the cancer or tumor and (b) detecting a loss of wild-type EP300 expression, e.g., expression from an EP300 allele not harboring the EP300 loss of function mutation in the cancer or tumor, wherein the cancer or tumor is identified as sensitive to CREBBP inhibition therapy, if the cancer or tumor harbors an EP300 loss of function mutation and exhibits loss of wild-type EP300 expression. In some embodiments, the loss of wild-type EP300 expression is a decrease of EP300 expression level of at least 50%, at least 60%, at least 70%, at least 75%, at least 80%, at least 90%, at least 95%, at least 97%, at least 98%, or at least 99% of EP300 expression, e.g., as compared to the EP300 expression level of a normal cell of the same tissue of origin as the cancer or tumor. In some embodiments, an EP300 loss of function is a complete abolishment of EP300 expression. Suitable methods for detecting the EP300 loss of function mutation, and suitable methods for detecting the expression, or absence thereof, of wild-type EP300 are provided herein or otherwise known to those of skill in the art. The disclosure is not limited in this respect.

### CREBBP Antagonists

In some embodiments, CREBBP inhibition therapy comprises administration of a CREBBP antagonist to a subject. For example, in some embodiments, CREBBP inhibition therapy comprises a CREBBP antagonist which decreases the level and/or activity of a CREBBP gene or gene product.

In some embodiments, a CREBBP antagonist comprises a polypeptide, nucleic acid, saccharide, lipid, small molecule, metal, or combination thereof. For example, in some embodiments, a CREBBP antagonist comprises an antibody or antigen-binding portion thereof (e.g., that specifically binds to a CREBBP gene product). In some embodiments, a CREBBP antagonist comprises a nucleic acid (e.g., an oligonucleotide, for example that acts to decrease production or translation of a CREBBP message (e.g., a primary transcript, a splice product), and/or a gene therapy agent (e.g., an agent that replaces or modifies a CREBBP gene or gene product). In some embodiments, a CREBBP antagonist comprises a small molecule agent (e.g., an organic compound with a molecular mass of less than 1,500 daltons). In some embodiments, the CREBBP antagonist is soluble in water. In some embodiments, the CREBBP antagonist is formulated in a tablet or in an injectable composition.

In some embodiments, a CREBBP antagonist targets, binds or inhibits the HAT domain of a CREBBP gene product. In some embodiments, a CREBBP antagonist targets, binds or inhibits the bromodomain of a CREBBP gene product. In some embodiments, the CREBBP antagonist also targets, binds, or inhibits the activity of at least one additional protein, e.g., EP300.

In some embodiments, CREBBP inhibition therapy induces reduction in tumor volume. In some embodiments, a reduction in tumor volume is the result of apoptosis. In some embodiments, a reduction in tumor volume is the result of necrosis.

Small Molecule Agents

In some embodiments, a CREBBP antagonist as described herein comprises a small molecule agent, e.g., an organic compound with a molecular mass of less than 1,500 daltons, less than 1,000 daltons, less than 900 daltons, less than 750 daltons, or less than 500 daltons. In some embodiments, a CREBBP antagonist as described herein is an inhibitor of histone acetyl transferase domains.

In some embodiments, a CREBBP antagonist is a small molecule of TABLE 2, or a pharmaceutically acceptable salt, hydrate, enantiomer or stereoisomer thereof:

TABLE 2

| # | Formula |
|---|---|
| 1 | 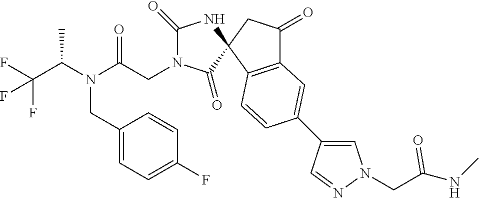 [SMILES: CNC(=O)Cn1cc(-c2ccc3c(c2)C(=O)C[C@]32NC(=O)N(CC(=O)N(Cc3ccc(F)cc3)[C@@H](C)C(F)(F)F)C2=O)cn1] |
| 2 | 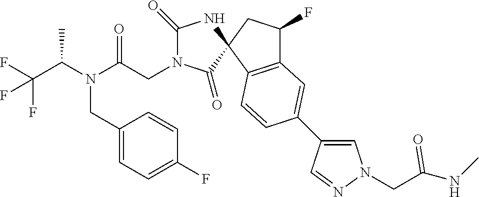 [SMILES: CNC(=O)Cn1cc(-c2ccc3c(c2)[C@H](F)C[C@]32NC(=O)N(CC(=O)N(Cc3ccc(F)cc3)[C@@H](C)C(F)(F)F)C2=O)cn1] |
| 3 | 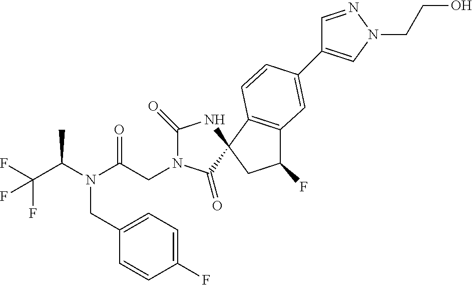 [SMILES: C[C@@H](N(Cc1ccc(F)cc1)C(=O)CN1C(=O)N[C@@]2(C[C@H](F)c3cc(-c4cnn(CCO)c4)ccc32)C1=O)C(F)(F)F] |

In some embodiments, a CREBBP antagonist is a small molecule of TABLE 3, or a pharmaceutically acceptable salt, hydrate, enantiomer or stereoisomer thereof:

TABLE 3

| # | Formula |
|---|---|
| 4 | 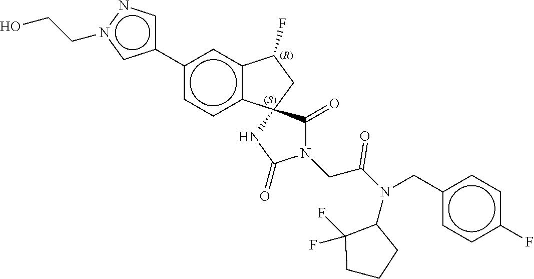 |
| 5 | 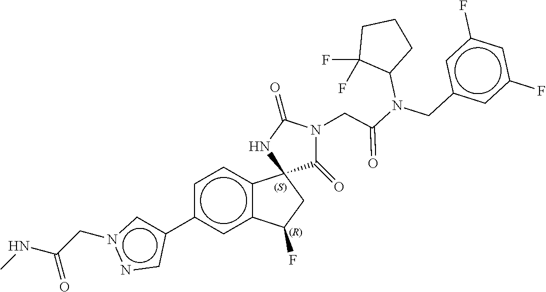 |
| 6 | 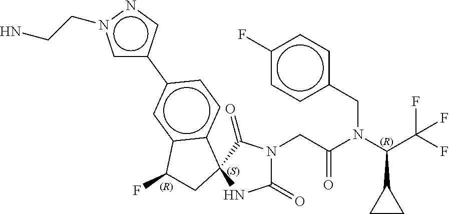 |
| 7 | 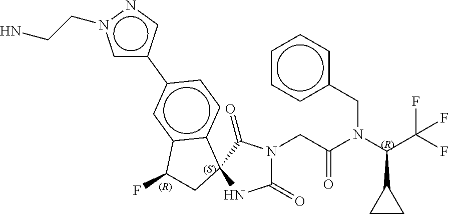 |
| 8 | 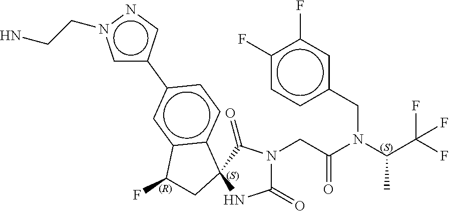 |
| 9 | 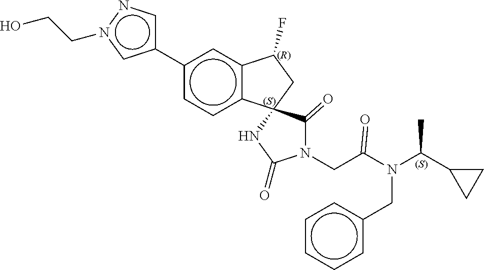 |
| 10 | 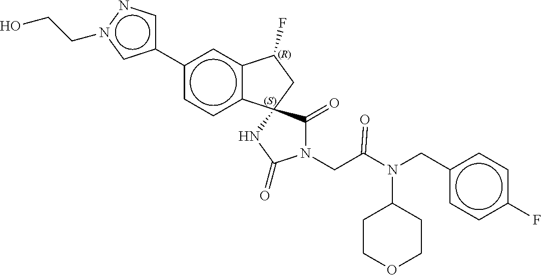 |
| 11 | 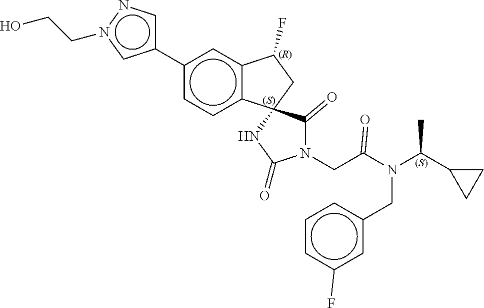 |
| 12 | 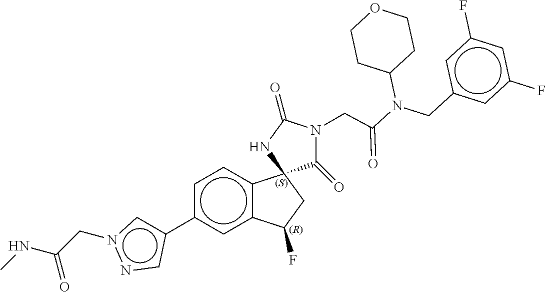 |

Additional suitable CREBBP antagonists will be apparent to those of ordinary skill in the art based on the present disclosure. Exemplary CREBBP antagonists suitable for use in some embodiments of this disclosure include, without limitation, those reported in International Patent Application PCT/US2015/050877, published under International Publication Number WO 2016/044694 A1; International Patent Application PCT/US2015/051028, published under International Publication Number WO 2016/044770 A1; International Patent Application PCT/US2015/051029, published under International Publication Number WO 2016/044771 A1; International Patent Application PCT/US2015/051040, published under International Publication Number WO 2016/044777 A1; International Patent Application PCT/CN2015/091614, published under International Publication Number WO 2016/055028 A1; International Patent Application PCT/US2014/060147, published under International Publication Number WO 2015/054642 A9; and International Patent Application PCT/US2015/062794, published under International Publication Number WO 2016/086200 A1; the entire contents of each of which are incorporated herein by reference.

Additional small molecule agents that may act as CREBBP antagonists and are suitable for use in some embodiments of this disclosure will be apparent to those of ordinary skill in the art, and non-limiting examples of such CREBBP antagonists include those reported in Taylor et al. ACS Med Chem Lett. 2016 Mar. 15; 7(5):531-6; Emami et al. Proc Natl Acad Sci USA. 2004; 101:12682-7; Guidez et. al. Mol. Cell Biol. 2005, 5552, 2012, 77, 9044; Chandregowda et al. Eur. J. Med. Chem. 2009, 44:2711-19; Secci et al. Bioorg Med Chem. 2014 Mar. 1; 22(5):1680-9; Bowers et al. Chem Biol. 2010 May 28; 17(5):471-82; Milite et al. J Med Chem. 2015 Mar. 26; 58(6):2779-98 Gajer et al. Oncogenesis. 2015 Feb. 9; Rooney et al. Angew Chem Int Ed Engl. 2014 Jun. 10; 53(24):6126-30; and Falk et al. J Biomol Screen December 2011 vol. 16 no. 10 1196-1205, the entire contents of each of which are incorporated herein by reference.

Additional suitable CREBBP antagonists will be apparent to those of ordinary skill in the art. The disclosure is not limited in this respect.

Nucleic Acid Agents

A variety of modalities are known and understood in the art for which an antagonistic therapeutic agent comprises a nucleic acid (e.g., an oligonucleotide or polynucleotide). For example, among others, antagonistic nucleic acid therapeutic agents can include sense or antisense nucleic acid agents, gene therapy agents, or gene editing agents.

In some embodiments, a sense or antisense nucleic acid agent includes siRNA, shRNA or miRNA. In some embodiments, a sense or antisense nucleic acid agent does not include siRNA. In some embodiments, an antagonistic therapeutic nucleic acid agent is a gene therapy agent. In some embodiments, a gene therapy agent comprises an agent, e.g., a DNA, or RNA, that modifies the level or activity of a gene or gene product. In some embodiments, a gene therapy agent comprises a sense or antisense nucleic acid in a vector system. In some embodiments, a vector system may comprisefor example, a vector and a lipid nucleic acid delivery system or a viral vector comprised in a viral envelope that is capable of introducing the vector into a target celle. In some embodiments, a gene editing agent can be an agent comprising a CRISPR/Cas system (Sternberg et al. Nature. 2014 Mar. 6; 507(7490):62-7; O'Connell et al. Nature. 2014 Dec. 11; 516(7530):263-6; Mali et al. Science. 2013 Feb. 15; 339(6121):823-6). In some embodiments, a gene editing agent can be an agent comprising a TALENs (Boch J et al. Annual Review of Phytopathology 48: 419-36; Boch J et al. Nature Biotechnology 29 (2): 135-6; Christian, M et al. Genetics 186 (2): 757-61). In some embodiments, a gene editing agent can be an agent comprising a zinc finger nuclease (Urnov et al. Nature. 2005 Jun. 2; 435(7042):646-51; Miller et al. Nat Biotechnol. 2007 July; 25(7):778-85; Hockemeyer et al. Nat Biotechnol. 2009 September; 27(9):851-7). In some embodiments, a CRISPR/Cas system, a TALEN system, or a ZFN system is targeted to a genomic sequence encoding a CREBBP protein in a target cell, e.g., a cancer cell, resulting in a loss-of-function mutation in the CREBBP protein in the target cell.

Polypeptide Agents

In some embodiments, a CREBBP antagonist as described herein comprises a polypeptide agent. In some embodiments, a polypeptide agent may be a recombinant polypeptide that can decrease level and/or activity of a CREBBP gene or gene product. In some embodiments, a polypeptide agent may bind the CREBBP gene product. In some embodiments, a polypeptide agent may be an antibody or an antigen-binding fragment thereof (e.g., a Fab, or an scFV). In some embodiments, a polypeptide agent may bind to and decrease the activity of polypeptides or nucleic acids that increase level of activity of CREBBP.

### Pharmaceutical Compositions

A CREBBP antagonist, e.g., a CREBBP antagonist provided herein, can be administered to a subject, e.g., to a human patient, alone, e.g., in the form of a pharmaceutically acceptable salt, a solvated or hydrated form of a CREBBP antagonist or a salt of a CREBBP antagonist, and any polymorph or crystal form of a CREBBP antagonist, including any polymorph or crystal form of a salt, solvate and/or hydrate form of a CREBBP antagonist, or in a pharmaceutical composition, e.g., where the CREBBP antagonist is admixed with a suitable carrier or excipient. A pharmaceutical composition typically comprises or can be administered at a dose sufficient to treat or ameliorate a disease or condition in the recipient subject, e.g., to treat or ameliorate a cancer as described herein. Accordingly, a pharmaceutical composition is formulated in a manner suitable for administration to a subject, e.g., in that it is free from pathogens and formulated according to the applicable regulatory standards for administration to a subject, e.g., for administration to a human subject. As an example, a formulation for injection is typically sterile and essentially pyrogen-free.

A suitable CREBBP antagonist can also be administered to a subject as a mixture with other agents, e.g., with one or more additional therapeutic agent(s), e.g., in a suitably formulated pharmaceutical composition. For example, one aspect of the present disclosure relates to pharmaceutical compositions comprising a therapeutically effective dose of a CREBBP antagonist, or a pharmaceutically acceptable salt, hydrate, enantiomer or stereoisomer thereof; and a pharmaceutically acceptable diluent or carrier.

Techniques for formulation and administration of CREBBP antagonists may be found in references well known to one of ordinary skill in the art, such as Remington's “The Science and Practice of Pharmacy,” 21st ed., Lippincott Williams & Wilkins 2005, the entire contents of which are incorporated herein by reference.

Pharmaceutical compositions as provided herein are typically formulated for a suitable route of administration. Suitable routes of administration may, for example, include enteral administration, e.g., oral, rectal, or intestinal administration; parenteral administration, e.g., intravenous, intramuscular, intraperitoneal, subcutaneous, or intramedullary injection, as well as intrathecal, direct intraventricular, or intraocular injections; topical delivery, including eyedrop and transdermal; and intranasal and other transmucosal delivery, or any suitable route provided herein or otherwise apparent to those of ordinary skill in the art.

The pharmaceutical compositions provided herein may be manufactured, e.g., by mixing, dissolving, granulating, dragee-making, levigating, emulsifying, encapsulating, entrapping, or lyophilizing processes, or by any other suitable processes known to those of ordinary skill in the art.

Pharmaceutical compositions for use in accordance with the present invention may be formulated using one or more physiologically acceptable carriers comprising excipients and auxiliaries which facilitate processing of the CREBBP antagonist(s) into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants are used in the formulation appropriate to the barrier to be permeated. Such penetrants are generally known in the art.

For oral administration, a CREBBP antagonist can be formulated readily by combining the CREBBP antagonist with pharmaceutically acceptable carriers known in the art. Such carriers enable the CREBBP antagonist(s) provided to be formulated as tablets, pills, dragees, capsules, liquids, gels, syrups, slurries, suspensions and the like, for oral ingestion by a patient to be treated. Pharmaceutical preparations for oral use can be obtained by combining the CREBBP antagonist(s) with a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of CREBBP antagonist(s) doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredient(s), e.g., one or more suitable CREBBP antagonist(s), in admixture with filler such as lactose, binders such as starches, and/or lubricants such as talc or magnesium stearate and, optionally, stabilizers. In soft capsules, the CREBBP antagonist(s) may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, the CREBBP antagonist(s) for use according to the present disclosure are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the CREBBP antagonist(s) and a suitable powder base such as lactose or starch.

Suitable CREBBP antagonists can be formulated for parenteral administration by injection, e.g., bolus injection or continuous infusion. Formulations for injection may be presented in unit dosage form, e.g., in ampoules, or in multi-dose containers, and, in some embodiments, may contain an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the CREBBP antagonist(s) in water-soluble form. Additionally, suspensions of the CREBBP antagonist(s) may be prepared as appropriate injection suspensions, e.g., CREBBP antagonist(s), e.g., aquaeous or oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the CREBBP antagonist(s) to allow for the preparation of highly concentrated solutions.

Alternatively, the active ingredient(s), e.g., the CREBBP antagonist(s), may be in powder form for reconstitution before use with a suitable vehicle, e.g., sterile pyrogen-free water.

The CREBBP antagonists may also be formulated in rectal compositions such as suppositories or retention enemas, e.g., containing conventional suppository bases, such as cocoa butter or other glycerides.

In addition to the formulations described previously, a CREBBP antagonist may also be formulated as a depot preparation. Such long acting formulations may be administered by implantation (for example, subcutaneously or intramuscularly or by intramuscular injection). Thus, for example, a CREBBP antagonist may be formulated with suitable polymeric or hydrophobic materials (for example as an emulsion in an acceptable oil) or ion exchange resins, or as sparingly soluble derivatives (for example, as a sparingly soluble salt).

Alternatively, other delivery systems for hydrophobic pharmaceutical CREBBP antagonists may be employed. Liposomes and emulsions are examples of delivery vehicles or carriers for hydrophobic drugs. Certain organic solvents such as dimethysulfoxide also may be employed. Additionally, a CREBBP antagonist may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the CREBBP antagonist(s) for a few hours, a few days, a few weeks, or a few months, e.g., up to over 100 days.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers, such as polyethylene glycols.

Additional suitable pharmaceutical compositions and processes and strategies for formulating a suitable CREBBP antagonist will be apparent to the skilled artisan based on the present disclosure. The disclosure is not limited in this respect.

### Methods of Treatment

Some aspects of this disclosure provide methods for modulating protein acetylation, e.g., histone acetylation, in a subject in need thereof by administering a CREBBP inhibitor to the subject in an amount sufficient to modulate acetylation of a target protein, e.g., a histone acetylated by CREBBP activity. In some embodiments, the subject is a subject having or diagnosed with a cancer or a precancerous condition. In some embodiments, the subject harbors a loss-of-function mutation in an EP300 gene or expresses a mutant EP300 gene product.

Provided herein are methods of treating, preventing or alleviating a symptom of conditions and diseases, such as cancers and precancerous conditions, the course of which can be influenced by modulating the acetylation status of histones or other proteins that are acetylated by CREBBP, wherein said acetylation status is mediated at least in part by the activity of CREBBP. Modulation of the acetylation status of histones can in turn influence the level of expression of target genes activated by acetylation, and/or target genes suppressed by acetylation.

For example, some aspects of the invention provide methods for treating or alleviating a symptom of cancer or precancerous condition. In some embodiments, the method comprises the step of administering to a subject having a cancer or a precancerous condition a CREBBP antagonist, e.g., in the form of a pharmaceutical composition, at a therapeutically effective amount. In some embodiments, the subject harbors a mutant EP300 gene or expresses a mutant EP300 gene product. In some embodiments, the mutation in the mutant EP300 gene or the mutant EP300 gene product is a loss-of-function mutation. In some embodiments, the subject, or a target cell in the subject, e.g., a cancer cell, expresses less than 50%, less than 40%, less than 30%, less than 25%, less than 20%, less than 10%, less than 5%, less than 2.5%, less than 2%, less than 1%, less than 1%, or less than 0.1% of EP300 activity as compared to a suitable reference EP300 activity, e.g., an EP300 activity measured or expected in a healthy subject or cell, or in a subject or cell not harboring a loss-of-function mutation in an EP300 gene or gene product, or expressing a wild type EP300 gene or gene product.

In some embodiments, the subject harbors an EP300 mutant comprising a mutation in its KIX domain, in its bromodomain, or in its HAT domain. In some embodiments, the subject harbors a mutation in one or more of the following residues in the EP300 protein sequence provided in SEQ ID NO: 3, or of a residue equivalent thereof: V5, R86, K291, T329, R397, G711, P802, P1081, Q993, G1042, R1055, C1201, R1234, C1385, D1399, Y1414, A1437, Y1467, K1468, K1488, W1509, R1645, S1650, S1754, Q1874, R1950, Q2023, and Q2306. For example, in some embodiments, the subject harbors one or more of the following mutations in the EP300 protein sequence provided in SEQ ID NO: 3, or a functionally equivalent mutation: a V5L, T329R, P802L, P1081S, C1201Y, C1385Y, D1399N, D1399Y, Y1414C, A1437V, W1509C, 51650Y, Q1874E, R1950G, or Q2306E substitution; a K291fs, R1234fs, K1468fs, K1488fs or Y1467fs frameshift mutation; a R86*, R397*, Q993*, G1042*, R1055*, R1645*, S1754*, or Q2023* truncation; or a splice variation at G711. In some embodiments, the subject harbors an EP300 mutation provided herein, e.g., anywhere in the specification, in Table 4, or in any of the drawings, or any combination of such mutations.

In some embodiments, the CREBBP inhibitor inhibits histone acetyltransferase activity of CREBBP. In some embodiments, the CREBBP inhibitor selectively inhibits histone acetyltransferase activity of CREBBP.

In some embodiments, the subject is diagnosed with a disease or disorder known to be associated with a dysregulation of histone acetylation, e.g., with a dysfunction, of EP300 and/or CREBBP. In some embodiments, the subject is diagnosed with a disease or disorder and has been found to harbor an EP300 loss-of-function mutation. In some embodiments, the subject has been diagnosed with a cancer.

Dysregulated histone acetylation has been reported to be involved in aberrant expression of certain genes in cancers and other diseases. CREBBP antagonists described herein can be used to treat such histone acetylation-associated diseases, e.g., to inhibit CREBBP-mediated histone acetylation in affected cells, tissues, or subjects.

Modulators of histone acetylation can be used for modulating cell proliferation, e.g., of cells harboring a mutation resulting in aberrant histone acetylation, or for inducing cell death in cells depending on CREBBP histone acetylation for survival or proliferation, e.g., in cells with loss-of-function in an EP300 gene or gene product sequence. Accordingly, diseases that may be treated with CREBBP antagonists include hyperproliferative diseases, such as benign cell growth and malignant cell growth (cancer), e.g., in hypoproliferative diseases harboring an EP300 loss-of-function mutation.

Exemplary cancers that may be treated with the CREBBP antagonists provided herein include, without limitation, EP300 mutant cancers, e.g., lymphomas, including non-Hodgkin lymphoma, follicular lymphoma (FL) and diffuse large B-cell lymphoma (DLBCL); melanoma; and leukemia, including CML; Acute Lymphoblastic Leukemia; Acute Myeloid Leukemia; Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bone Cancer, osteosarcoma/Malignant Fibrous Histiocytoma; Brain Stem Glioma; Brain Tumor; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Breast Cancer; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cervical Cancer; Childhood Cancers; Chronic Lymphocytic Leukemia; Chronic Myelogenous Leukemia; Chronic Myelogenous Leukemia, Hairy Cell; Chronic Myeloproliferative Disorders; Colon Cancer; Colorectal Cancer; Cutaneous T-Cell Lymphoma, see Mycosis Fungoides and Sezary Syndrome; Endometrial Cancer; Esophageal Cancer; Ewing's Family of Tumors; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Leukemia; Lip and Oral Cavity Cancer; Liver Cancer, Adult (Primary); Liver Cancer, Childhood (Primary); Lung Cancer, Non-Small Cell; Lung Cancer, Small Cell; Lymphoma, Primary Central Nervous System; Macroglobulinemia, Waldenstrom's; Malignant Fibrous Histiocytoma of Bone/Osteosarcoma; Medulloblastoma; Melanoma; Merkel Cell Carcinoma; Mesothelioma; Mesothelioma, Adult Malignant; Metastatic Squamous Neck Cancer with Occult Primary; Multiple Endocrine Neoplasia Syndrome; Multiple Myeloma; Multiple Myeloma/Plasma Cell Neoplasm Mycosis Fungoides; Myelodysplastic Syndromes; Myelodysplastic/Myeloproliferative Diseases; Myeloid Leukemia, Adult Acute; Myeloid Leukemia, Childhood Acute; Myeloproliferative Disorders, Chronic; Nasal Cavity and Paranasal Sinus Cancer; Nasopharyngeal Cancer; Neuroblastoma; Non-Hodgkin's Lymphoma; Non-Hodgkin's Lymphoma During Pregnancy; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Low Malignant Potential Tumor; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Prostate Cancer; Rectal Cancer; Retinoblastoma; Rhabdomyosarcoma; Salivary Gland Cancer; Sarcoma, Ewing's Family of Tumors; Sarcoma, Soft Tissue; Sarcoma, Uterine; Sezary Syndrome; Skin Cancer; Skin Cancer (non-Melanoma); Small Intestine Cancer; Soft Tissue Sarcoma; Squamous Cell Carcinoma, see Skin Cancer (non-Melanoma); Squamous Neck Cancer with Occult Primary, Metastatic; Stomach (Gastric) Cancer; Testicular Cancer; Thymoma; Thymoma and Thymic Carcinoma; Thyroid Cancer; Transitional Cell Cancer of the Renal Pelvis and Ureter; Trophoblastic Tumor, Gestational; Unknown Primary Site, Cancer of; Unusual Cancers of Childhood; Urethral Cancer; Uterine Cancer, Endometrial; Uterine Sarcoma; Vaginal Cancer; Visual Pathway and Hypothalamic Glioma; Vulvar Cancer; Waldenstrom's Macroglobulinemia; Wilms' Tumor; and Women's Cancers. Exemplary precancerous conditions that can be treated with CREBBP antagonists include EP300 mutant hyperproliferative diseases, e.g., myelodisplastic syndrome (MDS; formerly known as preleukemia).

Any other disease in which histone acetylation mediated by CREBBP plays a role, and associated with EP300 loss-of-function, may be treatable or preventable using compounds and methods described herein.

### Administration

In some embodiments, an active agent for use in accordance with the present disclosure is formulated, dosed, and/or administered in a therapeutically effective amount using pharmaceutical compositions and dosing regimens that are consistent with good medical practice and appropriate for the relevant agent(s) and subject(s). In principle, therapeutic compositions can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transdermal, or other kinds of administration involving physical breaching of a tissue of a subject and administration of the therapeutic composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular indication being treated, the clinical condition of a subject (e.g., age, overall health, prior therapy received and/or response thereto) the site of delivery of the agent, the nature of the agent (e.g. an antibody or other polypeptide-based compound), the mode and/or route of administration of the agent, the presence or absence of combination therapy, and other factors known to medical practitioners. For example, in the treatment of cancer, relevant features of the indication being treated may include, for example, one or more of cancer type, stage, location.

In some embodiments, one or more features of a particular pharmaceutical composition and/or of a utilized dosing regimen may be modified over time (e.g., increasing or decreasing the amount of active agent in any individual dose, increasing or decreasing time intervals between doses), for example in order to optimize a desired therapeutic effect or response (e.g., inhibition of a CREBBP gene or gene product).

In general, type, amount, and frequency of dosing of active agents in accordance with the present invention are governed by safety and efficacy requirements that apply when one or more relevant agent(s) is/are administered to a mammal, preferably a human. In general, such features of dosing are selected to provide a particular, and typically detectable, therapeutic response as compared to what is observed absent therapy.

In the context of the present invention, an exemplary desirable therapeutic response may involve, but is not limited to, inhibition of and/or decreased tumor growth, tumor size, metastasis, one or more of the symptoms and side effects that are associated with a tumor, as well as increased apoptosis of cancer cells, therapeutically relevant decrease or increase of one or more cell marker or circulating markers. Such criteria can be readily assessed by any of a variety of immunological, cytological, and other methods that are disclosed in the literature.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of the CREBBP antagonist and/or an additional therapeutic agent at increasing doses. Consequently, the relative amounts of the each agent within a pharmaceutical composition may also vary, for example, each composition may comprise between 0.001% and 100% (w/w) of the corresponding agent.

In some embodiments, a “therapeutically effective amount” or “therapeutically effective dose” is an amount of a CREBBP antagonist, or a combination of two or more CREBBP antagonists, or a combination of a CREBBP antagonist with one or more additional therapeutic agent(s), which inhibits, totally or partially, the progression of the condition or alleviates, at least partially, one or more symptoms of the condition. In some embodiments, a therapeutically effective amount can be an amount which is prophylactically effective. In some embodiments, an amount which is therapeutically effective may depend upon a patient's size and/or gender, the condition to be treated, severity of the condition and/or the result sought. In some embodiments, a therapeutically effective amount refers to that amount of a CREBBP antagonist that results in amelioration of at least one symptom in a patient. In some embodiments, for a given patient, a therapeutically effective amount may be determined by methods known to those of skill in the art.

In some embodiments, toxicity and/or therapeutic efficacy of CREBBP antagonists can be determined by standard pharmaceutical procedures in cell cultures or experimental animals, e.g., for determining the maximum tolerated dose (MTD) and the ED₅₀ (effective dose for 50% maximal response). Typically, the dose ratio between toxic and therapeutic effects is the therapeutic index; in some embodiments, this ratio can be expressed as the ratio between MTD and ED₅₀. Data obtained from such cell culture assays and animal studies can be used in formulating a range of dosage for use in humans.

In some embodiments, dosage may be guided by monitoring a CREBBP antagonist's effect on one or more pharmacodynamic markers of enzyme inhibition (e.g., histone acetylation or target gene expression) in diseased or surrogate tissue. For example, cell culture or animal experiments can be used to determine the relationship between doses required for changes in pharmacodynamic markers and doses required for therapeutic efficacy can be determined in cell culture or animal experiments or early stage clinical trials. In some embodiments, dosage of a CREBBP antagonist lies preferably within a range of circulating concentrations that include the ED₅₀ with little or no toxicity. In some embodiments, dosage may vary within such a range, for example depending upon the dosage form employed and/or the route of administration utilized. The exact formulation, route of administration and dosage can be chosen by the individual physician in view of the patient's condition. In the treatment of crises or severe conditions, administration of a dosage approaching the MTD may be required to obtain a rapid response.

In some embodiments, dosage amount and/or interval may be adjusted individually, for example to provide plasma levels of an active moiety which are sufficient to maintain, for example a desired effect, or a minimal effective concentration (MEC) for a period of time required to achieve therapeutic efficacy. In some embodiments, MEC for a particular CREBBP antagonist can be estimated, for example, from in vitro data and/or animal experiments. Dosages necessary to achieve the MEC will depend on individual characteristics and route of administration. In some embodiments, high pressure liquid chromatography (HPLC) assays or bioassays can be used to determine plasma concentrations.

In some embodiments, dosage intervals can be determined using the MEC value. In certain embodiments, CREBBP antagonists should be administered using a regimen which maintains plasma levels above the MEC for 10-90% of the time, preferably between 30-90% and most preferably between 50-90% until the desired amelioration of a symptom is achieved. In other embodiments, different MEC plasma levels will be maintained for differing amounts of time. In cases of local administration or selective uptake, the effective local concentration of the drug may not be related to plasma concentration.

One of skill in the art can select from a variety of administration regimens and will understand that an effective amount of a particular CREBBP antagonist may be dependent on the subject being treated, on the subject's weight, the severity of the affliction, the manner of administration and/or the judgment of the prescribing physician.

### Combination Therapy

In some embodiments, a CREBBP antagonist can be used in combination with another therapeutic agent to treat diseases such as cancer. In some embodiments, a CREBBP antagonist, or a pharmaceutical composition comprising a CREBBP inhibition therapy agent as described herein can optionally contain, and/or be administered in combination with, one or more additional therapeutic agents, such as a cancer therapeutic agent, e.g., a chemotherapeutic agent or a biological agent. An additional agent can be, for example, a therapeutic agent that is art-recognized as being useful to treat the disease or condition being treated by the CREBBP antagonist, e.g., an anti-cancer agent, or an agent that ameliorates a symptom associated with the disease or condition being treated. The additional agent also can be an agent that imparts a beneficial attribute to the therapeutic composition (e.g., an agent that affects the viscosity of the composition). For example, in some embodiments, CREBBP inhibition therapy is administered to a subject who has received, is receiving, and/or will receive therapy with another therapeutic agent or modality (e.g., with a chemotherapeutic agent, surgery, radiation, or a combination thereof).

Some embodiments of combination therapy modalities provided by the present disclosure provide, for example, administration of a CREBBP antagonist and additional agent(s) in a single pharmaceutical formulation. Some embodiments provide administration of a CREBBP antagonist and administration of an additional therapeutic agent in separate pharmaceutical formulations.

Examples of chemotherapeutic agents that can be used in combination with a CREBBP inhibition therapy agent described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, BMS-936559, RG7446/MPDL3280A, MEDI4736, tremelimumab, or others known in the art), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, a CREBBP antagonist is administered to a subject in need thereof in combination with another agent for the treatment of cancer, either in the same or in different pharmaceutical compositions. In some embodiments, the additional agent is an anticancer agent. In some embodiments, the additional agent affects (e.g., inhibits) histone modifications, such as histone acetylation or histone methylation. In certain embodiments, an additional anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Velcade® and Zevalin™); small molecules (such as Tykerb®); corticosteroids (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

The additional agents that can be used in combination with CREBBP antagonist therapy as set forth above are for illustrative purposes and not intended to be limiting. The combinations embraced by this disclosure, include, without limitation, one or more CREBBP antagonists as provided herein or otherwise known in the art, and at least one additional agent selected from the lists above or otherwise provided herein. The CREBBP antagonists can also be used in combination with one or with more than one additional agent, e.g., with two, three, four, five, or six, or more, additional agents.

In some embodiments, treatment methods described herein are performed on subjects for which other treatments of the medical condition have failed or have had less success in treatment through other means, e.g., in subjects having a cancer refractory to standard-of-care treatment. Additionally, the treatment methods described herein can be performed in conjunction with one or more additional treatments of the medical condition, e.g., in addition to or in combination with standard-of-care treatment. For instance, the method can comprise administering a cancer regimen, e.g., nonmyeloablative chemotherapy, surgery, hormone therapy, and/or radiation, prior to, substantially simultaneously with, or after the administration of a CREBBP inhibition therapy agent described herein, or composition thereof. In certain embodiments, a subject to which a CREBBP inhibition therapy agent described herein is administered can also be treated with antibiotics and/or one or more additional pharmaceutical agents.

Identification and/or Characterization of CREBBP Antagonists

Some aspects of the present disclosure provide technologies for identifying and/or characterizing CREBBP antagonists.

For example, in some embodiments, a candidate CREBBP antagonist is contacted with a system comprising, at least, CREBBP, and an assay is performed to detect binding of the candidate CREBBP antagonist to CREBBP. In some embodiments, the candidate is identified as a CREBBP antagonist if a candidate binds to CREBBP. In some embodiments, a candidate CREBBP antagonist is contacted with a system comprising, at least, CREBBP and an assay is performed to detect modulation in CREBBP level and/or activity. In some embodiments, a the candidate is identified as a CREBBP antagonist if modulation of CREBBP level and/or activity is detected in the presence of the candidate. In some embodiments, the system further comprises a CREBBP substrate (e.g., a histone or a fragment or complex thereof) and an acetyl donor. In some embodiments, a candidate CREBBP antagonist is contacted with a system comprising CREBBP, a CREBBP substrate, and an acetyl donor. In some embodiments, an assay is performed to detect the level of CREBBP substrate acetylation. In some embodiments, a candidate is identified as a CREBBP antagonist if the level of CREBBP substrate acetylation in the system is greater in the absence of the candidate CREBBP antagonist than in the presence of the candidate CREBBP antagonist.

### Detecting Inhibition

In some embodiments, function or capability of a CREBBP inhibition therapy agent to decrease the level and/or activity of a CREBBP gene or gene product is evaluated, e.g., in vitro or in vivo. In some embodiments, function or capability of a CREBBP inhibition therapy agent to decrease the level and/or activity of a CREBBP gene or gene product is evaluated relative to an appropriate reference. In some embodiments, an appropriate reference is a historical reference, a population-based reference or a subject-specific reference. In some embodiments, the evaluation is based on a biological sample, e.g., a sample obtained from a subject. In some embodiments, the sample used to evaluate function or capability of a CREBBP inhibition therapy agent is also used to determine the mutation state of the tumor, as described elsewhere herein.

In some embodiments, function or capability of a CREBBP inhibition therapy agent is evaluated by measuring apoptosis of tumor cells. In some embodiments, apoptosis of tumor cells is measured by cleavage of PARP. In some embodiments, function or capability of a CREBBP inhibition therapy agent is evaluated by modulation of MYC expression. In some embodiments, function or capability of a CREBBP inhibition therapy agent is evaluated by measuring acetylation of histones.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXEMPLIFICATION

### Example 1: Tumor Cell Lines Sensitive to Loss of CREBBP

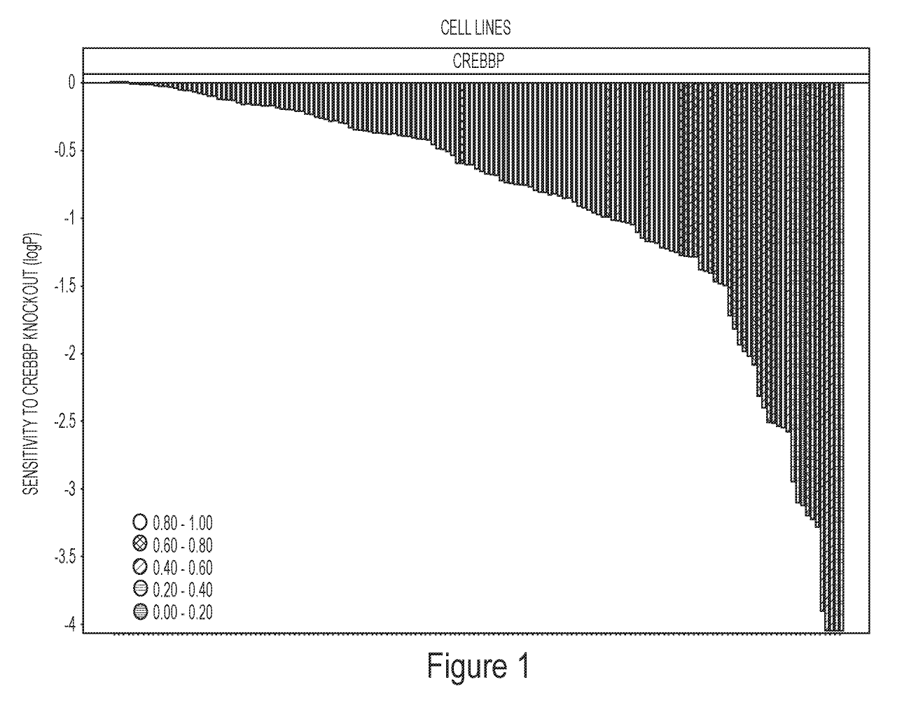

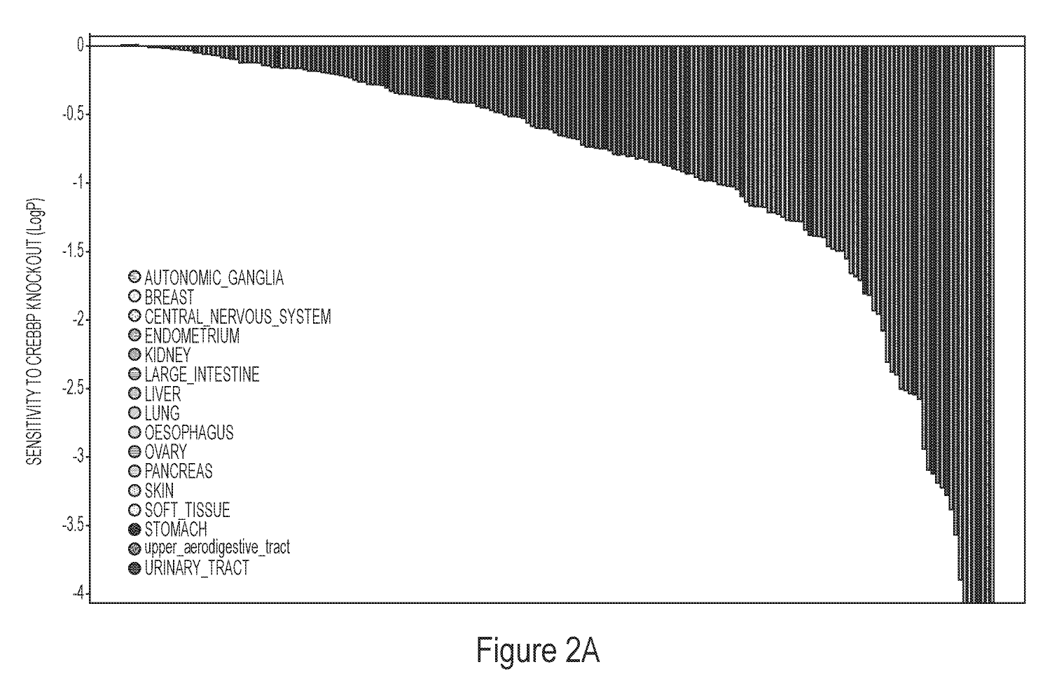

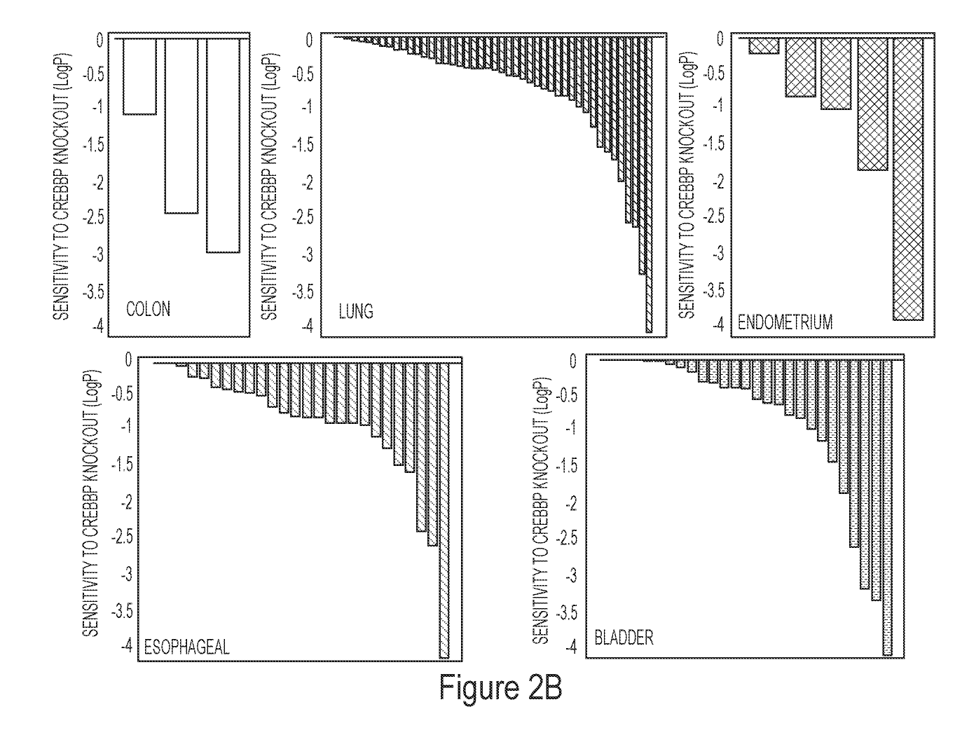

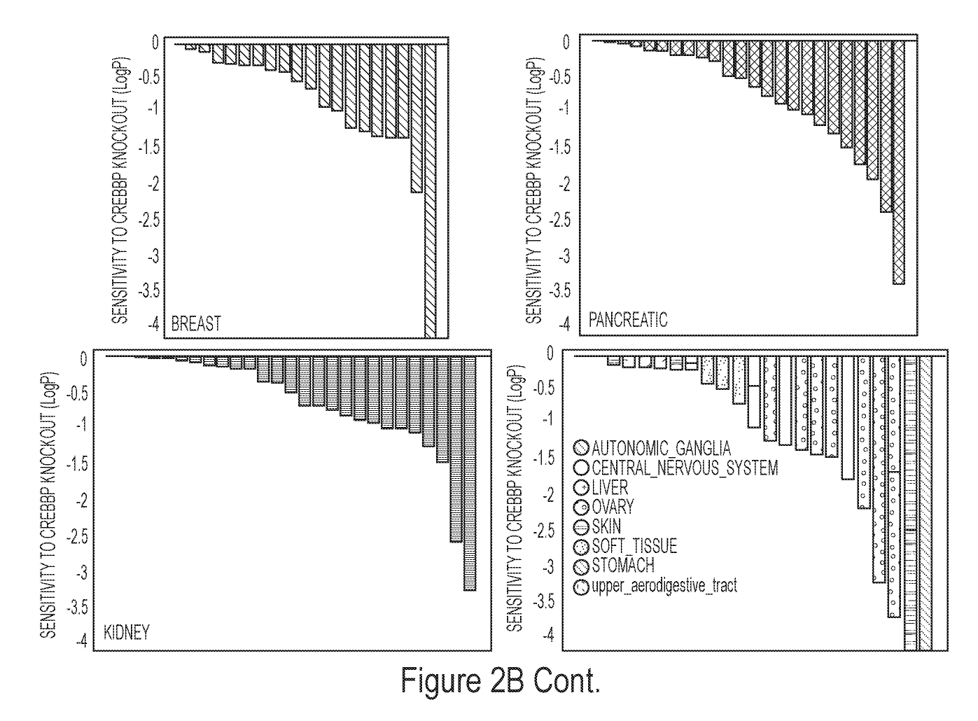

The present Example demonstrates that tumor cell lines derived from a wide range of tissues are sensitive to CREBBP loss of activity. A custom library of sgRNAs targeting epigenetic related genes was introduced into a panel of tumor cell lines expressing CRISPR protein. The screen was performed in a similar manner to that as previously described by Shalem et al. Science. 2014 Jan. 3; 343(6166): 84-87 and Wang et al. Science. 2014 Jan. 3; 343(6166):80-4. Significance of the sensitivity of the cell lines to the loss of function in each gene was calculated using the Redundant siRNA activity (RSA) score, and is represented herein as Log P, as previously described by Birmingham et al., Nat Methods. 2009 August; 6(8): 569-575). FIG. 1 demonstrates a distribution of sensitivity to CREBBP loss of function in the panel of tumor cell lines. FIG. 2A demonstrates that sensitivity to CREBBP loss is observed in tumor cell lines derived from numerous and varied tissue types. FIG. 2B further details the wide range of tumor types sensitive to the loss of CREBB. Moreover, FIG. 2B shows that amongst an individual tissue type there is a distribution of sensitivity to CREBBP loss.

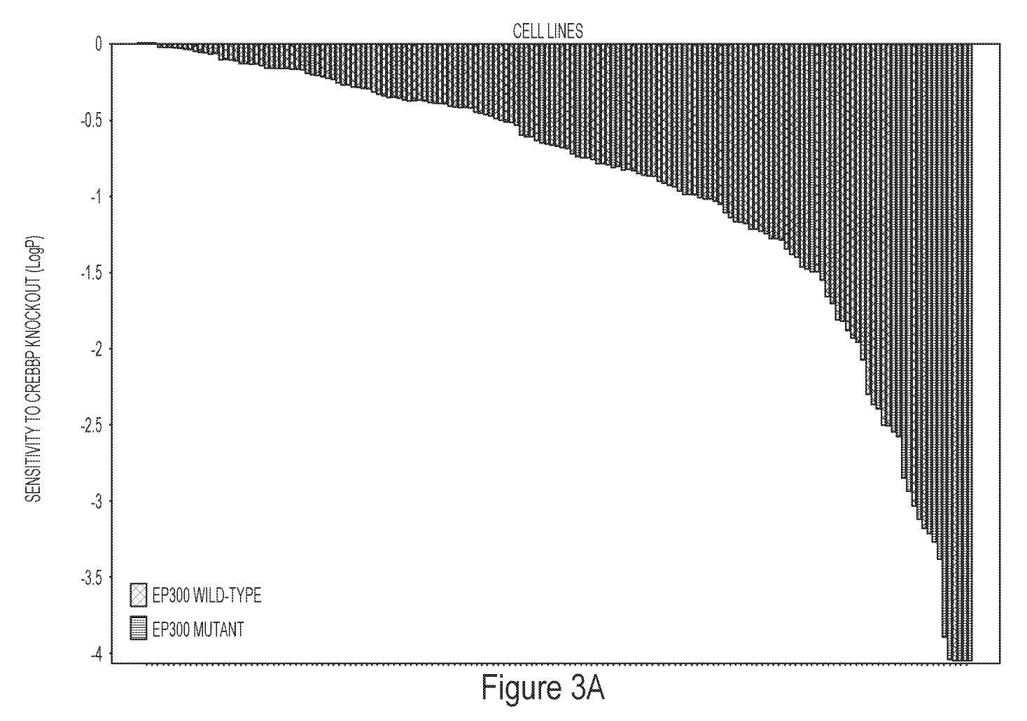

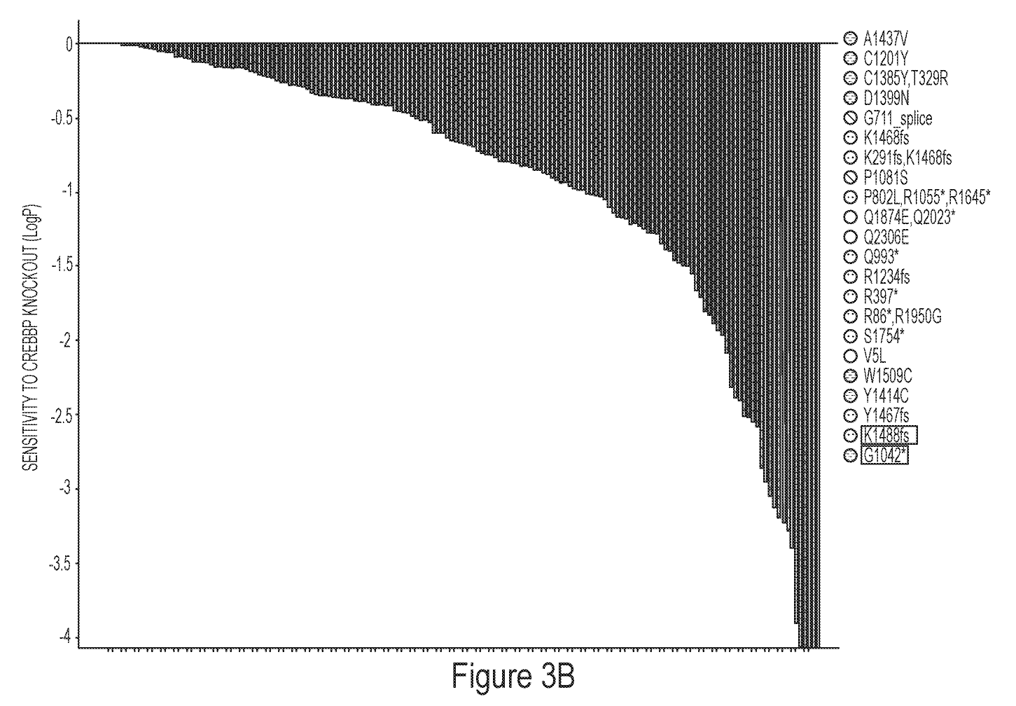

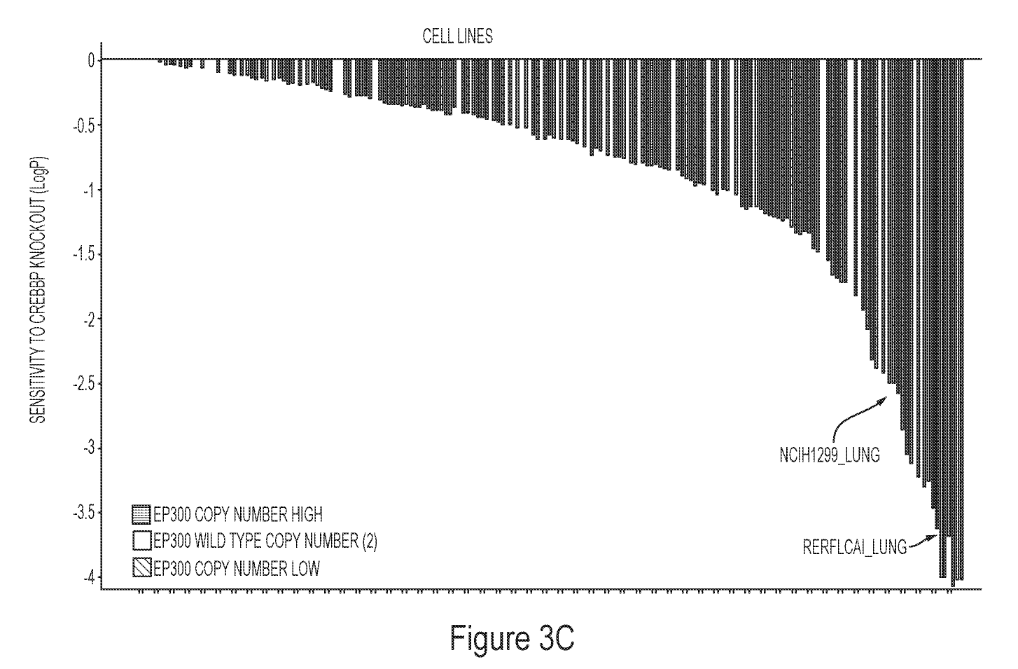

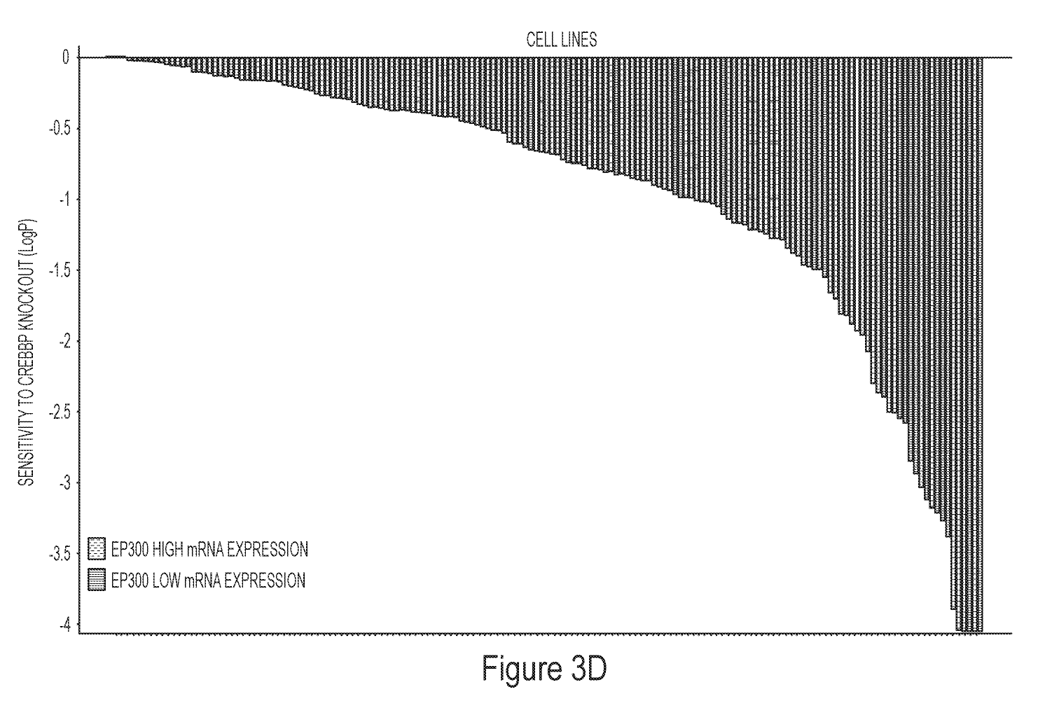

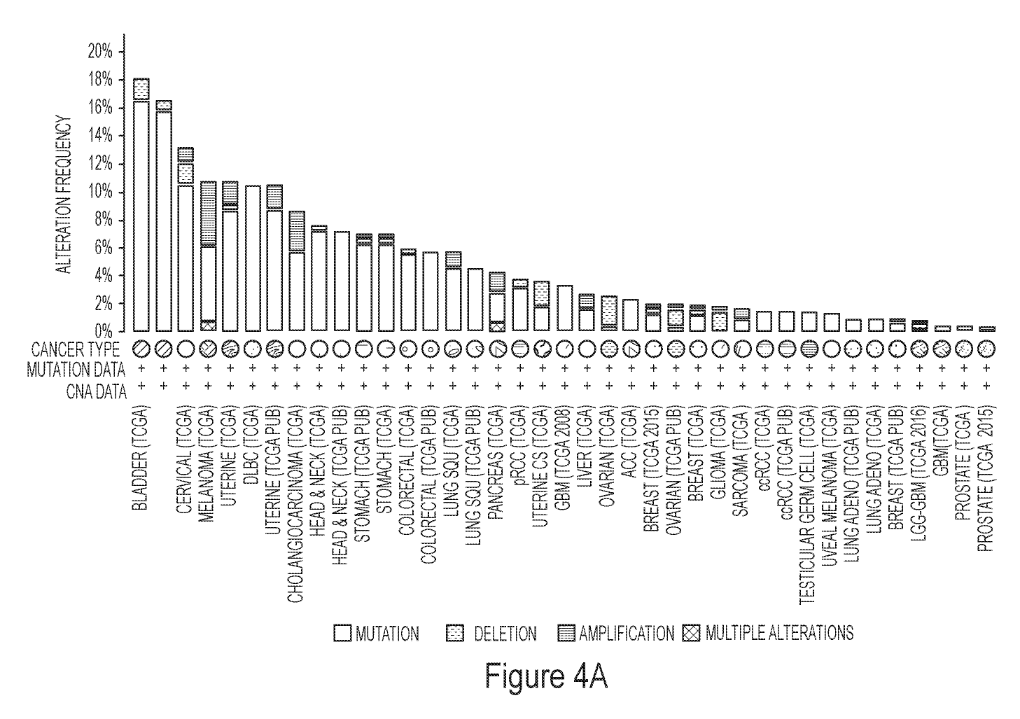

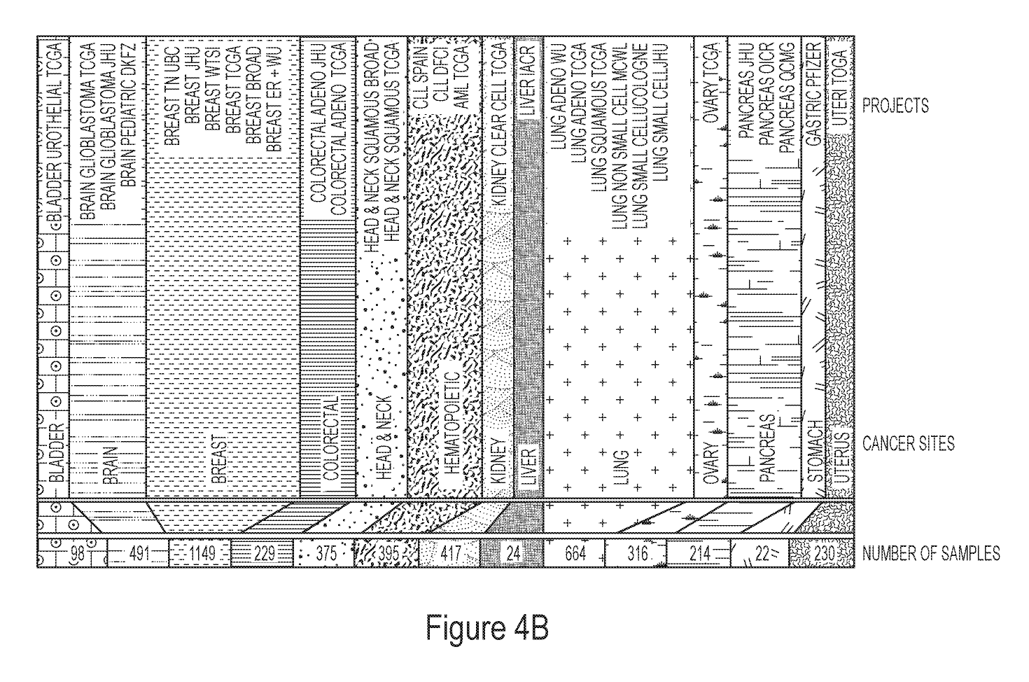

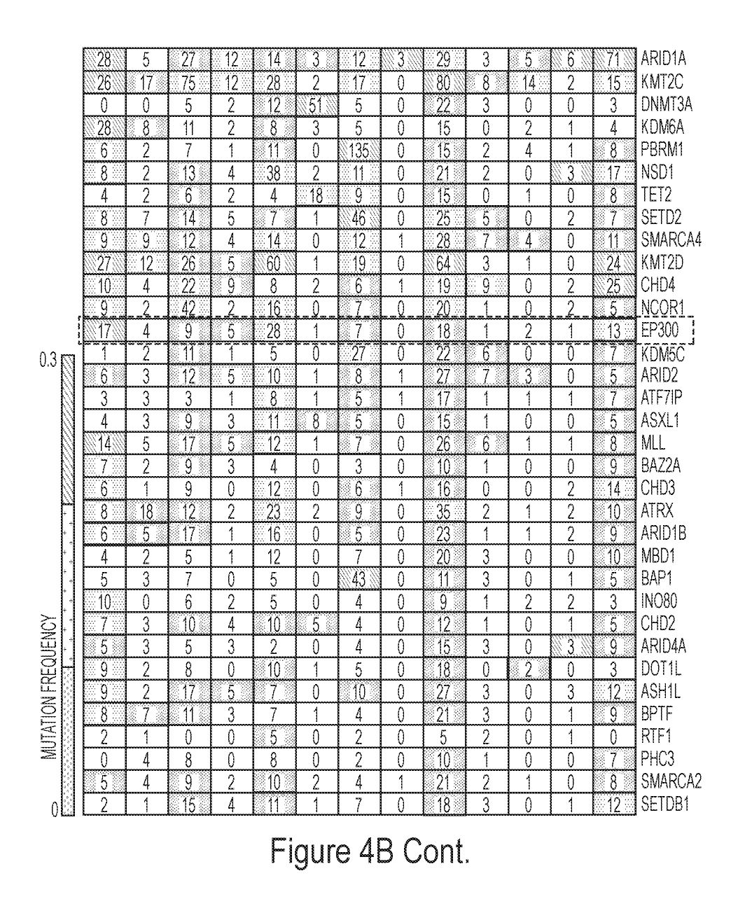

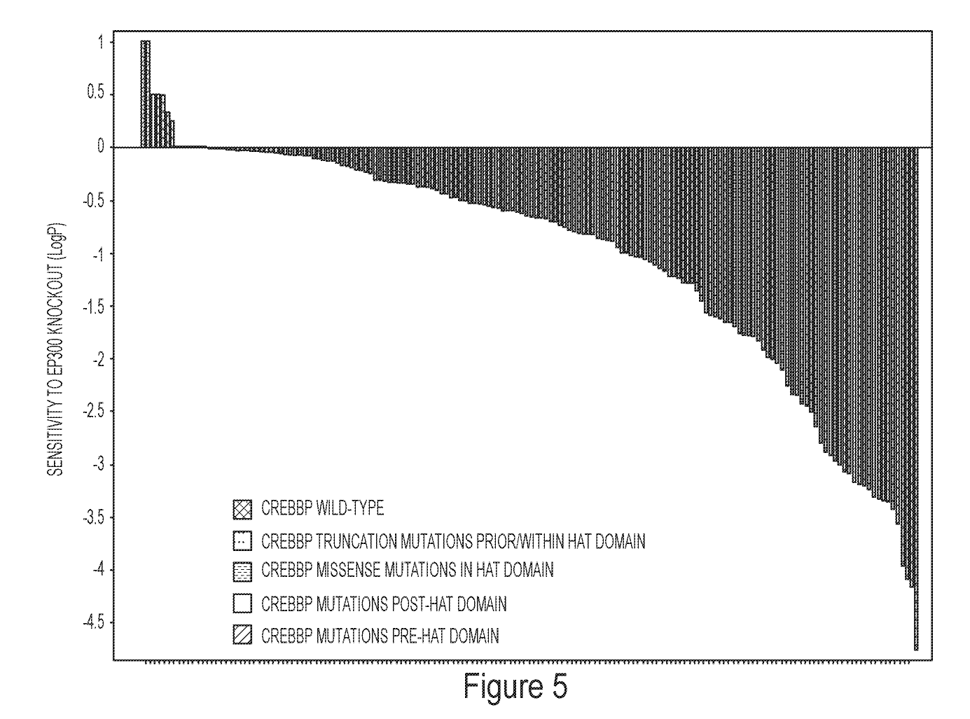

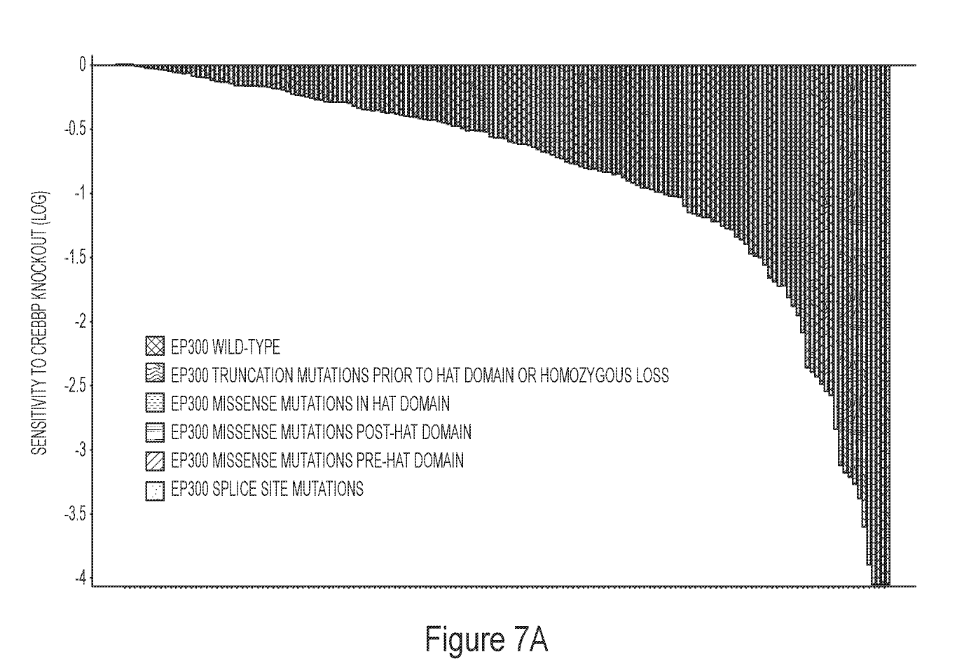

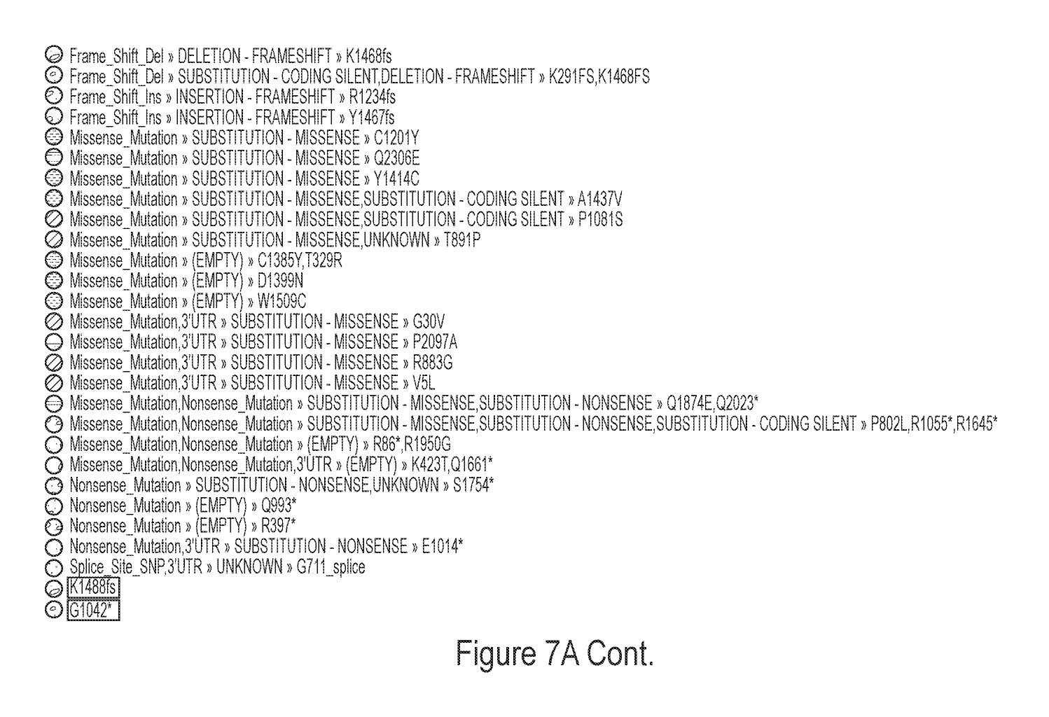

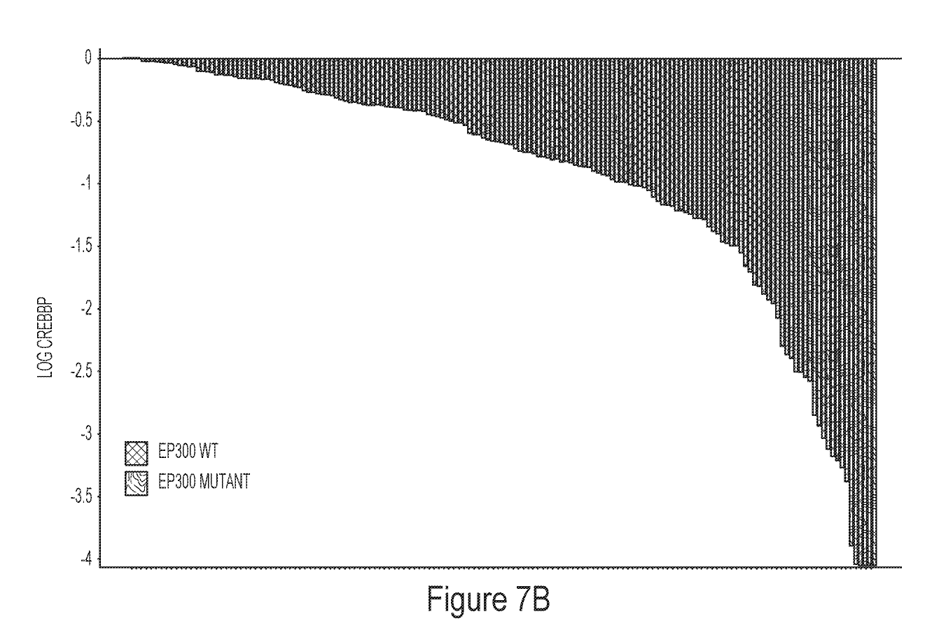

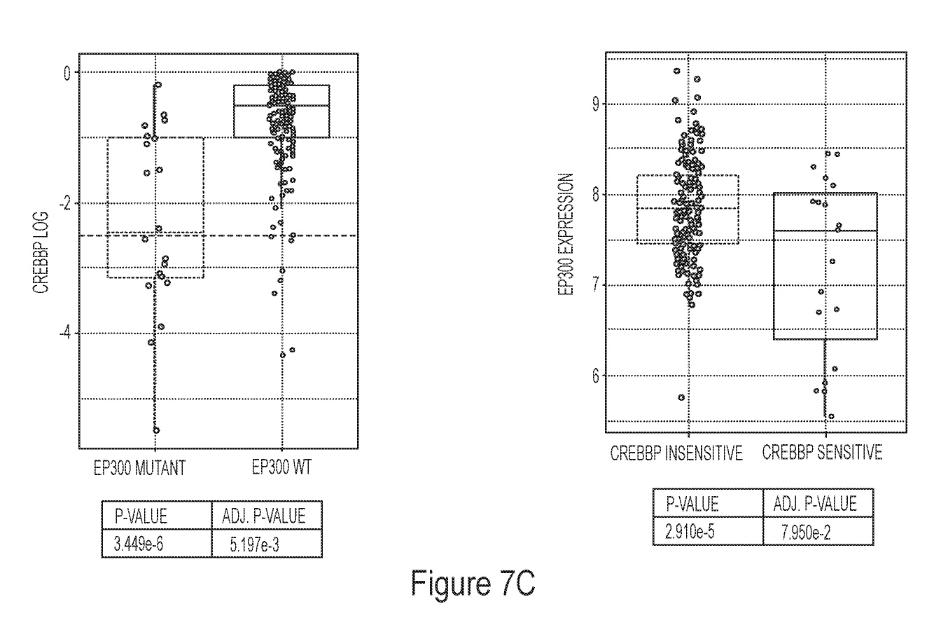

The tumor cell lines used in the sgRNA screen were then evaluated for their EP300 mutation or expression level status. A large number of tumor cell lines harboring EP300 mutation were found to be among the most sensitive to CREBBP loss (FIG. 3A and FIG. 7B). FIG. 7C provides statistical analysis to further confirm the sensitivity to CREBBP inhibition. A wide variety of tumors have been identified as harboring EP300 mutations (FIG. 4). Interestingly, those cell lines identified in our screen as being sensitive to EP300 loss do not harbor corresponding mutations in CREBBP (FIG. 5).

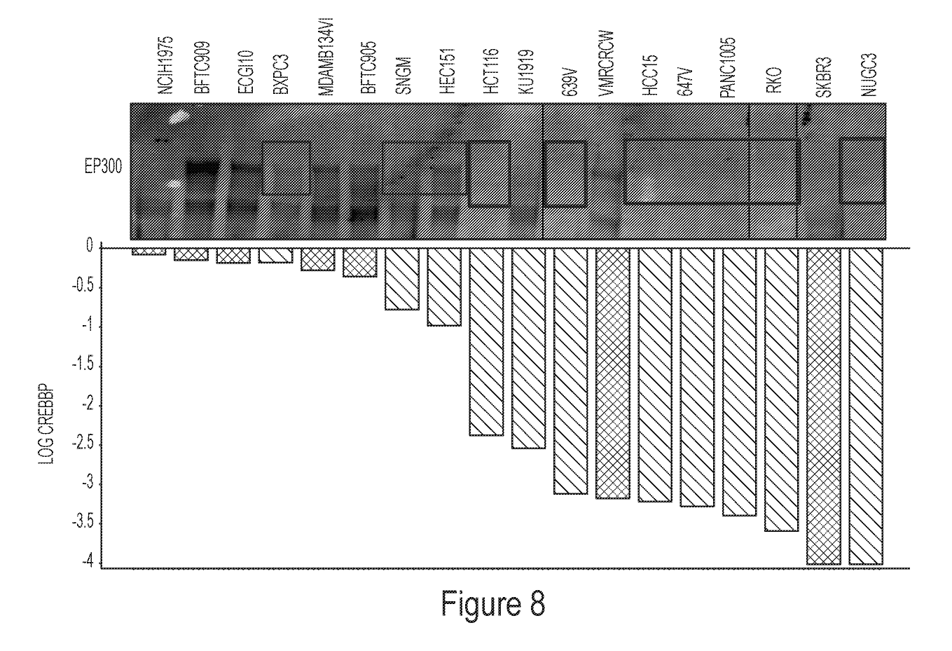

EP300 mutations can be found in numerous locations throughout the gene or gene product. FIGS. 3B and 7A provide further detail of the type of EP300 mutation and their location in the gene or gene product relative to the HAT domain. FIG. 8 further demonstrates types of EP300 mutations. Specifically, FIG. 8 shows cell lines with EP300 truncation mutations resulting in loss of expression of EP300 though a wild type allele may still be intact. These data further confirm that loss of EP300 expression is predictive of sensitivity to CREBBP inhibition. DNA copy variation and expression level of EP300 among the screened tumor cell lines are shown in FIGS. 3C and 3D.

### Example 2: CREBBP Inhibition of Cells Bearing EP300 Mutations

The present Example documents effects of inhibiting CREBBP in cell lines bearing EP300 mutations. Available tumor cell lines or cultures of cells derived from tumor biopsies are evaluated for their EP300 mutation status. Those cell lines or cultures found to harbor one or more mutations in the EP300 gene or gene product are then contacted with exemplary CREBBP antagonists. Subsequently, the effect of a CREBBP antagonists on the cell line or culture is assessed. Reduction of cell line or culture growth and/or induction of apoptosis in the cells demonstrates the effectiveness of CREBBP inhibition therapy on tumor cells harboring one or more EP300 mutations. Additionally, through this process novel CREBBP antagonists are identified.

### Example 3: CREBBP Inhibition of Xenografts with Tumors Characterized by EP300 Mutations

The present Example documents effects of inhibiting CREBBP in xenografts of tumors harboring EP300 mutations. Tumor cells, derived from cell cultures or derived from a subjects tumor, harboring EP300 mutations are grafted into appropriate mouse models. Subsequent to establishment of a tumor a CREBBP inhibition therapy agent is administered to the mouse harboring the xenografted tumor. The volume of the tumor, or other aspects characterizing tumor growth or status, are assessed. Reduction of stasis of tumor growth demonstrates the effectiveness of CREBBP inhibition on tumors harboring one or more EP300 mutations. Additionally, through this process novel CREBBP inhibition therapy agents are identified.

### Example 4: CREBBP Inhibition Therapy of Human Subjects Bearing Tumors Characterized By EP300 Mutations

The present Example describes administration of CREBBP inhibition therapy to particular human subjects—specifically, those bearing tumors that are characterized by EP300 mutations. Some aspects of the present Example describe the identification of such subjects. A sample is obtained from a subject. The sample comprises a tumor, a biopsy of a tumor, circulating tumor cells, or other sample which comprises tumor derived nucleic acid or polypeptide. The nucleic acid or polypeptide is isolated from the sample and evaluated for EP300 mutation status by a method described herein or others known in the art. Subjects whose tumors are found to harbor EP300 mutations are administered CREBBP inhibition therapy alone or in combination with other therapeutics.

### REFERENCES

All publications, patents, patent applications, patent publications, and database entries (e.g., sequence database entries) mentioned herein, e.g., in the Background, Summary, Drawings, Detailed Description, Examples, and/or References sections, are hereby incorporated by reference in their entirety as if each individual publication, patent, patent application, patent publication, and database entry was specifically and individually incorporated herein by reference. In case of conflict, the present application, including any definitions herein, shall control.

### EQUIVALENTS AND SCOPE

Those skilled in the art will recognize, or be able to ascertain using no more than routine experimentation, many equivalents to the specific embodiments of the invention(s) described herein. The scope of the present disclosure is not intended to be limited to the above Description, but rather is as set forth in the following claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments, in which exactly one member of the group is present, embodiments, in which two or more members of the group are present, and embodiments, in which all of the group members are present. For purposes of brevity, those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood, in general, where an embodiment, product, or method is referred to as comprising particular elements, features, or steps, that embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges, e.g., in some embodiments, to the tenth of the unit of the lower limit of the range, unless the context clearly dictates otherwise. For the purpose of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range. For the purpose of brevity, the subranges have not been individually spelled out herein, but it will be understood that each of these subranges is provided herein and may be specifically claimed or disclaimed.

In addition, it is to be understood that any particular embodiment of the present invention may be explicitly excluded from any one or more of the claims. Where ranges are given, any value or subrange within the range may explicitly be excluded from any one or more of the claims. Any embodiment, element, feature, application, or aspect of the compositions and/or methods of the invention, can be excluded from any one or more claims. For the purpose of brevity, all of the embodiments in which one or more elements, features, purposes, or aspects is excluded are not set forth explicitly herein.

We claim:

1. A method of treating cancer comprising a step of:
administering CREBBP inhibition therapy to a subject in need thereof, wherein the subject has or is diagnosed with a cancer.

2. The method of claim 1, wherein the cancer is characterized by at least one mutation in EP300.

3. The method of claim 1, wherein the method comprises administering a CREBBP antagonist to the subject in a therapeutically effective amount.

4. The method of claim 1, wherein the method further comprises obtaining the sample from the subject.

5. The method of claim 2, wherein at least one mutation is detected in an EP300 gene product in a sample obtained from the subject.

6. The method of claim 2, wherein the method further comprises detecting the at least one mutation in an EP300 gene product in a sample obtained from the subject.

7. The method of claim 1, wherein the cancer comprises a tumor.

8. The method of claim 3, wherein the tumor is a solid tumor.

9. The method of claim 4, wherein the tumor is a tumor of the colon, lung, esophagus, bladder, breast, endometrium, uterus, cervix, kidney, central nervous system, liver, ovary, pancreas, skin, stomach, head and neck, or upper respiratory tract.

10. The method of claim 1, wherein the cancer is a hematologic malignancy.

11. The method of claim 6, wherein the cancer is diffuse large B-Cell lymphoma

12. The method of claim 1, wherein administering the CREBBP antagonist decreases the level and/or activity of a CREBBP gene product.

13. The method of claim 12, wherein the level and/or activity of the CREBBP gene product is decreased by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% as compared to the level and/or activity in the absence of the CREBBP antagonist.

14. The method of claim 12, wherein the CREBBP inhibition therapy comprises administration of a CREBBP antagonist selected from nucleic acid agents, small molecule agents, or polypeptide agents.

15. The method of claim 14, wherein a nucleic acid agent CREBBP antagonist comprises CRISPR/Cas, siRNA, shRNA, or miRNA.

16. The method of claim 14, wherein a polypeptide agent CREBBP antagonist comprises an antibody or fragment thereof.

17. The method of claim 1, wherein the mutant EP300 is characterized by decreased level and/or activity of an EP300 gene product relative to an appropriate reference.

18. The method of claim 17, wherein the level and/or activity of the mutant EP300 is decreased by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to the level and/or activity of an appropriate reference.

19. The method of claim 17, wherein the appropriate reference is the level and/or activity of wild-type EP300.

20. The method of claim 1, wherein the mutant EP300 comprises a frame shift mutation, a splice variant, a missense mutation, a nonsense mutation, an insertion, a deletion, or a combination thereof.

21. The method of claim 1, wherein the mutant EP300 comprises a mutation resulting in a V5L, C1201Y, C1385Y, T329R, D1399N, A1437V, splice variation at G711, K1468fs, K1488fs, K291fs, R1234fs, Y1467fs, P1081S, P802L, G1042*, R1055*, R1645*, Q1874E, Q2023*, Q2306E, Q993*, R397*, R86*, R1950G, S1754*, W1509C, or Y1414C substitution, or a combination thereof.

22. The method of claim 1, wherein the mutant EP300 comprises a mutation resulting in a a G30V, K423T, R883G, T891P, P2097A, or a E1014*, or Q1661* truncation.

23. The method of claim 1, wherein the mutant EP300 comprises a mutation listed in Table 4, or a combination of the mutations listed in Table 4.

24. The method of claim 1, wherein the mutant EP300 is characterized by a reduction in DNA copy number.

25. The method of claim 1, wherein the mutant EP300 is characterized by a disruption of the HAT domain of EP300.

26. The method of claim 1, wherein the mutant EP300 is characterized by a loss of the HAT domain of EP300.

27. The method of claim 1, wherein the mutant EP300 is characterized by a missense mutation.

28. The method of claim 27, wherein the missense mutation is within the HAT domain of EP300.

29. The method of claim 27, wherein the missense mutation is upstream of the HAT domain of EP300.

30. The method of claim 27, wherein the missense mutation is downstream of the HAT domain of EP300.

31. The method of claim 1, wherein the mutant form of EP300 is characterized by a truncation mutation.

32. The method of claim 28, wherein the truncation mutation is upstream of the HAT domain of EP300.

33. The method of claim 1, wherein the mutant form of EP300 is characterized by homozygous loss of the EP300 gene product.

34. The method of claim 1, wherein the CREBBP inhibition therapy leads to reduction of tumor volume.

35. The method of claim 31, wherein reduction in tumor volume is a result of apoptosis or necrosis of tumor cells.

36. The method of claim 1, wherein the subject has received or is receiving other cancer therapy.

37. A method of treating cancer, the method comprising a step of:
administering a CREBBP antagonist to a subject who has been diagnosed with the cancer by detecting presence in a sample from the subject of a mutant EP300.

38. The method of claim 37, wherein the method comprises administering a CREBBP antagonist to the subject in a therapeutically effective amount.

39. The method of claim 37, wherein the cancer comprises a tumor.

40. The method of claim 39, wherein the tumor is a solid tumor.

41. The method of claim 40, wherein the tumor is a tumor of the colon, lung, esophagus, bladder, breast, endometrium, uterus, cervix, kidney, central nervous system, liver, ovary, pancreas, skin, stomach, head and neck, or upper respiratory tract.

42. The method of claim 37, wherein the cancer is a hematologic malignancy.

43. The method of claim 42, wherein the cancer is diffuse large B-Cell lymphoma.

44. The method of claim 37, wherein administering the CREBBP antagonist decreases the level and/or activity of a CREBBP gene product.

45. The method of claim 44, wherein the level and/or activity of the CREBBP gene product is decreased by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% as compared to the level and/or activity in the absence of the CREBBP antagonist.

46. The method of claim 44, wherein the CREBBP inhibition therapy comprises administration of a CREBBP antagonist selected from nucleic acid agents, small molecule agents, or polypeptide agents.

47. The method of claim 46, wherein a nucleic acid agent CREBBP antagonist comprises CRISPR/Cas, siRNA, shRNA, or miRNA.

48. The method of claim 46, wherein a polypeptide agent CREBBP antagonist comprises an antibody or fragment thereof.

49. The method of claim 37, wherein the mutant EP300 is characterized by decreased level and/or activity of an EP300 gene product relative to an appropriate reference.

50. The method of claim 49, wherein the level and/or activity of the mutant EP300 is decreased by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% as compared to the level and/or activity of an appropriate reference.

51. The method of claim 49, wherein the appropriate reference is the level and/or activity of wild-type EP300.

52. The method of claim 37, wherein the mutant EP300 comprises a frame shift mutation, a splice variant, a missense mutation, a nonsense mutation, an insertion, a deletion, or a combination thereof.

53. The method of claim 37, wherein the mutant EP300 comprises a mutation resulting in a V5L, C1201Y, C1385Y, T329R, D1399N, A1437V, splice variation at G711, K1468fs, K1488fs, K291fs, R1234fs, Y1467fs, P1081S, P802L, G1042*, R1055*, R1645*, Q1874E, Q2023*, Q2306E, Q993*, R397*, R86*, R1950G, S1754*, W1509C, or Y1414C substitution, or a combination thereof.

54. The method of claim 37 wherein the mutant EP300 comprises a mutation resulting in a a G30V, K423T, R883G, T891P, P2097A, or a E1014*, or Q1661* truncation.

55. The method of claim 37, wherein the mutant EP300 comprises a mutation listed in Table 4, or a combination of the mutations listed in Table 4.

56. The method of claim 37, wherein the mutant EP300 is characterized by a reduction in DNA copy number.

57. The method of claim 37, wherein the mutant EP300 is characterized by a disruption of the HAT domain of EP300.

58. The method of claim 37, wherein the mutant EP300 is characterized by a loss of the HAT domain of EP300.

59. The method of claim 37, wherein the mutant EP300 is characterized by a missense mutation.

60. The method of claim 59, wherein the missense mutation is within the HAT domain of EP300.

61. The method of claim 59, wherein the missense mutation is upstream of the HAT domain of EP300.

62. The method of claim 59, wherein the missense mutation is downstream of the HAT domain of EP300.

63. The method of claim 37, wherein the mutant form of EP300 is characterized by a truncation mutation.

64. The method of claim 63, wherein the truncation mutation is upstream of the HAT domain of EP300.

65. The method of claim 37, wherein the mutant form of EP300 is characterized by homozygous loss of the EP300 gene product.

66. The method of claim 37, wherein the CREBBP inhibition therapy leads to reduction of tumor volume.

67. The method of claim 66, wherein reduction in tumor volume is a result of apoptosis or necrosis of tumor cells.

68. The method of claim 37, wherein the subject has received or is receiving other cancer therapy.

69. A method for identifying a CREBBP antagonist, the method comprising the steps of:
contacting a system comprising at least CREBBP, a CREBBP substrate,
and an acetyl donor with a candidate CREBBP antagonist; and
detecting acetylation of the CREBBP substrate.

70. The method of claim 69, wherein the CREBBP substrate is a histone.

71. The method of claim 69, wherein the candidate CREBBP antagonist is identified as a CREBBP antagonist if acetylation of the CREBBP substrate is less than acetylation of the CREBBP substrate in the absence of the candidate CREBBP antagonist.
